(19) United States
(12) Patent Application Publication
(10) Pub. No.: US 2024/0041840 A1
(43) Pub. Date: Feb. 8, 2024

(54) USE OF DEXPRAMIPEXOLE FOR THE TREATMENT OF MODERATE TO SEVERE ASTHMA

(71) Applicant: Areteia Therapeutics, Inc., Chapel Hill, NC (US)

(72) Inventor: Calman Philip PRUSSIN, Rockville, MD (US)

(73) Assignee: Areteia Therapeutics, Inc., Chapel Hill, NC (US)

(21) Appl. No.: 18/485,082

(22) Filed: Oct. 11, 2023

Related U.S. Application Data
(63) Continuation of application No. 17/395,059, filed on Aug. 5, 2021.
(60) Provisional application No. 63/061,226, filed on Aug. 5, 2020.
(60) Provisional application No. 63/136,933, filed on Jan. 13, 2021.
(60) Provisional application No. 63/147,024, filed on Feb. 8, 2021.
(60) Provisional application No. 63/174,938, filed on Apr. 14, 2021.

(51) Int. Cl. A61K 31/428; A61K 9/00; A61P 11/06; A61K 45/06

(52) U.S. Cl. CPC A61K 31/428; A61K 9/0053; A61P 11/06; A61K 45/06

(57) ABSTRACT

Disclosed herein are methods of treating moderate to severe asthma of the eosinophilic phenotype in a human subject in need thereof with a daily dose of about 75 mg to about 300 mg of dexpramipexole or pharmaceutical acceptable salt thereof, and treating severe asthma of the eosinophilic phenotype in a human subject in need thereof with a daily dose of about 150 mg to about 300 mg of dexpramipexole or pharmaceutical acceptable salt thereof.

### CROSS-REFERENCE TO RELATED APPLICATIONS

This application claims the benefit of U.S. Provisional Application No. 63/061,226, filed Aug. 5, 2020 and U.S. Provisional Application No. 63/136,933, filed Jan. 13, 2021 and U.S. Provisional Application No. 63/147,024, filed Feb. 8, 2021 and U.S. Provisional Application No. 63/174,938, filed Apr. 14, 2021. The disclosures of each of these applications are incorporated herein by reference.

### SUMMARY

Embodiments of the invention are directed to methods for treating moderate to severe asthma of the eosinophilic phenotype in a human subject in need thereof comprising orally administering to the subject a daily dose of about 75 mg to about 300 mg of dexpramipexole, or a pharmaceutically acceptable salt thereof, wherein the subject is already receiving at least two asthma medications, thereby treating the moderate to severe asthma of the eosinophilic phenotype in the human subject. In some embodiments, the at least two asthma medications are an inhaled corticosteroid (ICS) and a long-acting beta agonist (LABA). In embodiments described herein, moderate to severe asthma of the eosinophilic phenotype is treated by reducing one or more of the symptoms selected from the group consisting of level of absolute blood eosinophil, level of tissue eosinophils, amount of mucus plugs in the airways, level of sputum eosinophil peroxidase, level of nasal eosinophil peroxidase, level of pharyngeal eosinophil peroxidase, and combinations thereof. In embodiments described herein, moderate to severe asthma of the eosinophilic phenotype is treated by improving one or more of the symptoms selected from the group consisting of the frequency of asthma exacerbations, the use of oral corticosteroids, use of inhaled corticosteroids, use of long-acting beta agonist, use of short-acting beta agonist, use of rescue medications, the mean forced expiratory volume in 1 second (FEV₁), forced vital capacity (FVC), the score of the Asthma Control Questionnaire (ACQ), score on Asthma Control Test (ACT)™, score of Asthma Quality of Life Questionnaire (AQLQ), and a combination thereof.

### BRIEF DESCRIPTION OF THE DRAWINGS

FIG. 1 provides a depiction of the Study Design.

FIG. 2A graphically shows the geometric mean (SE) blood absolute eosinophil counts up to week 24. FIG. 2B graphically shows the eosinophil count ratio, corrected to placebo for week 12.

FIG. 3 demonstrates dexpramipexole improvement on FEV₁ in all dose groups.

FIG. 4 demonstrates dexpramipexole improvement on FVC in all dose groups.

FIG. 5 shows the significant FEV₁ increase at all post-baseline study time points seen in dexpramipexole subgroup with ≥50% AEC decrease (measured at week 12, hematologic responders).

FIG. 6 provides a comparison of the effects of dexpramipexole, benralizumab, and mepolizumab on FEV₁, lung function.

FIG. 7 show the greater FEV1 improvement in the subgroup with baseline AEC ≥400/μL.

FIG. 8 shows the significant tissue EPX lowering by dexpramipexole at Week 12.

FIG. 9 shows correlation between blood eosinophilic lowering and nasal tissue EPX lowering by dexpramipexole.

FIG. 10 shows the greater ACQ-6 improvement in subgroup with ≥50% AEC reduction.

FIG. 11 shows the greater ACQ-6 improvement in subgroup with ΔΔFEV₁ ≥100 mL.

### DETAILED DESCRIPTION

Dexpramipexole ((6R)-2-amino-4,5,6,7-tetrahydro-6-(propylamino) benzothiazole), is a synthetic aminobenzothiazole derivative with the following structure:

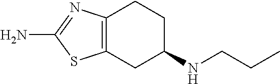

As used herein, dexpramipexole may be administered as a free base or a pharmaceutical acceptable salt, preferably the dihydrochloride salt. As used herein, dexpramipexole is 99.9% to 100% enantiomerically pure. The dexpramipexole contained in a pharmaceutical composition may have a chiral purity for dexpramipexole of at least 99.5%, preferably at least 99.6%, preferably at least 99.7%, preferably at least 99.8%, preferably at least 99.9%, preferably at least 99.95%, or more preferably at least 99.99%. In some embodiments, the chiral purity for dexpramipexole is 100%. In some embodiments, the composition has a chiral purity for dexpramipexole of 99.9% or greater. In some embodiments, the composition has a chiral purity for dexpramipexole of 99.95% or greater. In some embodiments, the composition has a chiral purity for dexpramipexole of 99.99% or greater. The high chiral purity of the pramipexole used herein, dexpramipexole, allows for therapeutic compositions that may have a wide individual and daily dose range.

All embodiments for amounts of, chiral purity of, and dosage form of dexpramipexole, or a pharmaceutically acceptable salt thereof, in the pharmaceutical compositions described herein are provided separately for the sake of brevity, but may be joined in any suitable combination.

Although any methods and materials similar or equivalent to those described herein can be used in the practice or testing of embodiments of the present invention, the exemplary methods, devices, and materials are now described.

In each of the embodiments described herein, the method may consist of orally administering a daily dose of about 75 mg to about 300 mg of dexpramipexole, or a pharmaceutically acceptable salt thereof. More preferably, the method may consist of orally administering a daily dose of about 150 mg to about 300 mg of dexpramipexole, or a pharmaceutically acceptable salt thereof. Dexpramipexole, or a pharmaceutically acceptable salt thereof, may be administered as 37.5 mg, 75 mg, or 150 mg twice per day

The term “comprising” means “including, but not limited to.” The term “consisting essentially of” means the method or composition includes the steps or components specifically recited, and may also include those that do not materially affect the basic and novel characteristics of the present invention. The term “consisting of” means the method or composition includes only the steps or components specifically recited. While various compositions and methods are described in terms of “comprising” various components or steps, in any embodiment, the composition or method can also utilize “consist essentially of” or “consist of” in reference to the described components or steps, but such embodiments are not explicitly listed and included for the sake of brevity, clarity, and efficiency.

Before the present compositions and methods are described, it is to be understood that this invention is not limited to the particular processes, compositions, or methodologies described, as these may vary. Moreover, the processes, compositions, and methodologies described in particular embodiments are interchangeable. Therefore, for example, a composition, dosage regimen, route of administration, and so on described in a particular embodiment may be used in any of the methods described in other particular embodiments. It is also to be understood that the terminology used in the description is for the purpose of describing the particular versions or embodiments only, and is not intended to the limit the scope of the present invention which will be limited only by the appended claims. Unless defined otherwise, all technical and scientific terms used herein have the same meanings as commonly understood by one of the ordinary skill in the art. Although any methods similar or equivalent to those describe herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg and ranges within, such as 2 mg-5 mg or 3 mg-5 mg, etc.

All percentages, parts and ratios are based upon the total weight of the oral compositions and all measurements made are at about 25° C., unless otherwise specified.

It must be noted that, as used herein, and in the appended claims, the singular forms “a”, “an” and “the” include plural reference unless the context clearly dictates otherwise.

As used herein, the term “about” means plus or minus 10% of the numerical value of the number with which it is being used. Therefore, about 50% means in the range of 45% to 55%.

“Administering” when used in conjunction with a therapeutic means to administer a therapeutic directly or indirectly into or onto a target tissue to administer a therapeutic to a patient whereby the therapeutic positively impacts the tissue to which it is targeted. “Administering” a composition may be accomplished by oral administration in any formulation currently known in the art. “Administering” may include the act of self-administration or administration by another person such as a health care provider.

As used herein, the term “baseline” refers to the status of the subject prior to the administration of and treatment with dexpramipexole, or pharmaceutically acceptable salt thereof; however, the subject may be receiving other medications for the treatment of asthma.

As used herein, the term “eosinophil” refers to an eosinophil granulocyte. In some embodiments, the term “eosinophil” refers to a human eosinophil progenitor (hEoP). In some embodiments, the term “eosinophil” refers to an eosinophil lineage-committed progenitor (EoP). In some embodiments, the term “eosinophil” refers to a human common myeloid progenitor (hCMP). In some embodiments, the term “eosinophil” refers to any combination of an eosinophil granulocyte, a human eosinophil progenitor (hEoP), an eosinophil lineage-committed progenitor (EoP), and a human common myeloid progenitor (hCMP). In some embodiments, the term “eosinophil” refers to a CD34+CD125+ progenitor cell. In some embodiments, the term “eosinophil” refers to an eosinophil residing in the bone marrow, in the systemic circulatory system, and/or in organ tissues, including the bone marrow, lungs, and airways. In some embodiments, the organ tissue is the lung, the skin, the heart, the brain, the eye, the gastrointestinal tract, the thymus, the spleen, the kidney, the bladder, the ear, the nose, the sinuses, the oral cavity, the upper respiratory tract, the bone marrow, or combinations thereof.

The term “improves” is used to convey that the present invention changes either the form, characteristics, structure, function and/or physical attributes of the tissue to which it is being provided, applied or administered. “Improves” may also refer to the overall physical state of an individual to whom an active agent has been administered. For example, the overall physical state of an individual may “improve” if one or more symptoms of the disease, condition or disorder are alleviated by administration of an active agent.

In each of the embodiments disclosed herein, the compounds and methods may be utilized with or on a subject in need of such treatment, which may also be referred to as “in need thereof” As used herein, the phrase “in need thereof” means that the subject has been identified as having a need for the particular method or treatment and that the treatment is being given to the subject for that particular purpose.

A human subject having “moderate to severe asthma of the eosinophilic phenotype” has an eosinophil level of at least 300 cells per microliter in the peripheral blood prior to any administration of dexpramipexole, or pharmaceutically acceptable salt thereof, and even if the subject is already receiving treatment with one or more asthma medications, for example, at least two asthma medications, such as an inhaled corticosteroid (ICS) and a long-acting beta agonist (LABA).

Any reference to symptoms, signs, or biomarkers are herein collectively referred to as “symptom.”

“Optional” or “optionally” may be taken to mean that the subsequently described structure, event or circumstance may or may not occur, and that the described includes instances where the event occurs and instances where it does not.

As used herein, the term “patient” and “subject” are interchangeable and may be taken to mean any living organism, which may be treated with compounds of the present invention. As such, the terms “patient” and “subject” may include, but is not limited to, any human. In some embodiments, the “patient” or “subject” is an adult, child, or infant. In some embodiments, the “patient” or “subject” is a human.

As used herein, the term “therapeutic” means an agent utilized to treat, combat, ameliorate, or prevent an unwanted disease, condition, or disorder of a patient.

The terms “therapeutically effective amount” or “therapeutic dose” is used herein are interchangeable and may refer to the amount of an active agent, pharmaceutical compound, or composition that elicits a clinical, biological, or medicinal response in a tissue, system, animal, individual, or human that is being sought by a researcher, veterinarian, medical doctor, or other clinical professional. A clinical, biological, or medical response may include, for example, inhibiting a disease, condition, or disorder in an individual that is experiencing or displaying the pathology or symptoms of the disease, condition, or disorder, or arresting further development of the pathology and/or symptoms of the disease, condition, or disorder, or ameliorating a disease, condition, or disorder in an individual that is experiencing or exhibiting the pathology or symptoms of the disease, condition, or disorder or reversing the pathology and/or symptoms experience or exhibited by the individual.

The term “prevent” may be taken to mean prophylaxis of a specific disorder, disease, or condition. In some embodiments, the term refers to preventing a disease, condition or disorder in an individual that may be predisposed to the disease, condition or disorder but does not yet experience or display pathology or symptoms of the disease, condition or disorder,

The term “treating” may be taken to mean alleviation of the symptoms associated with a specific disorder, disease, or condition and/or prevention of the worsening of symptoms associated with a specific disorder, disease, or condition. In some embodiments, the term refers to slowing or arresting the progression of the disorder, disease, or condition. In some embodiments, the term refers to alleviating the symptoms associated with the specific disorder, disease, or condition. In some embodiments, the term refers to alleviating the symptoms associated with the specific disorder, disease, or condition. In some embodiments, the term refers to restoring function which was impaired or lost due to a specific disorder, disorder or condition.

As used herein, the terms “enantiomers,” “stereoisomers,” and “optical isomers may be used interchangeably and refer to molecules which contain an asymmetric or chiral center and are mirror images of one another. Further, the terms “enantiomers,” “stereoisomers,” or “optical isomers” describe a molecule which, in a given configuration, cannot be superimposed on its mirror image.

As used herein, the terms “optically pure” or “enantiomerically pure” may be taken to indicate that a composition contains at least 99.95% of a single optical isomer of a compound. The term “enantiomerically enriched” may be taken to indicate that a least 51% of a composition in a single optical isomer or enantiomer. The term “enantiomeric enrichment” as used herein refer to an increase in the amount of one enantiomer as compared to the other. A “racemic” mixture of 2 amino 4,5,6,7 tetrahydro 6 (propylamino)benzothiazole is a mixture of about equal amounts of (6R) and (6S) enantiomers of 2 amino 4,5,6,7 tetrahydro 6 (propylamino)benzothiazole.

Throughout this disclosure, the word “pramipexole” will refer to (6S) enantiomer of 2 amino 4,5,6,7 tetrahydro 6 (propylamino)benzothiazole unless otherwise specified.

The term “pharmaceutical composition” shall mean a composition including at least one active ingredient, whereby the composition is amenable to investigation for a specified, efficacious outcome in a human. Those of ordinary skill in the art will understand and appreciate the techniques appropriate for determining whether an active ingredient has a desired efficacious outcome based upon the needs of the artisan. A pharmaceutical composition may, for example, contain dexpramipexole or a pharmaceutically acceptable salt of dexpramipexole as the active ingredient. Alternatively, a pharmaceutical composition may contain dexpramipexole or a pharmaceutically acceptable salt of dexpramipexole as the active ingredient.

“Pharmaceutically acceptable salt” is meant to indicate those salts which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 1 19, describes pharmaceutically acceptable salts in detail. A pharmaceutical acceptable “salt” is any acid addition salt, preferably a pharmaceutically acceptable acid addition salt, including, but not limited to, halogenic acid salts such as hydrobromic, hydrochloric, hydrofloric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p toluenesufonic, acetic, malic, fumaric, succinic, citric, benzonic gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di hydrohalogic, di sulfuric, di phosphoric or di organic acid salt. In all cases, the acid addition salt is used as an achiral reagent which is not selected on the basis of any expected or known preference for the interaction with or precipitation of a specific optical isomer of the products of this disclosure.

As used herein, the term “daily dose” refers to the amount of dexpramipexole, or pharmaceutically acceptable salt, per day that is administered to a patient. This amount can be administered in multiple unit doses or in a single unit doses, in a single time during the day or at multiple times during the day.

As used herein, the term “hematologic responder human subject” refers to a human subject with moderate to severe asthma that has a greater than or equal to 50% reduction in absolute blood eosinophil level from baseline after administration of dexpramipexole, or a pharmaceutically acceptable salt thereof.

Asthma is a heterogeneous disease, usually characterized by chronic airway inflammation. It is defined by the history of respiratory symptoms such as wheeze, shortness of breath, chest tightness, and cough that vary over time and in intensity, together with expiratory airflow limitation. Asthma is a condition in which the airways narrow due to airway smooth muscle constriction and mucosal swelling. The increase of mucus production in the airway lumen further contributes to obstructing airflow and worsening asthma symptoms. This can make breathing difficult and trigger coughing, wheezing and shortness of breath. For some people, asthma is a minor nuisance. For others, is a major problem that interferes with daily activities and that may lead to a life-threatening asthma attack or exacerbation. Current treatments cannot cure asthma, but its symptoms can be controlled in some patients.

Asthma signs and symptoms include: shortness of breath, chest tightness or pain, trouble sleeping caused by shortness of breath, coughing or wheezing (wheezing is a common sign of asthma in children), and exercise intolerance. Such signs and symptoms are typically worsened by viral upper respiratory infections, such as a cold or influenza. Asthmatics experience episodic exacerbations of their asthma, which consist of an increase in their asthma symptoms. Asthma exacerbations are a major cause of asthma morbidity and comprise a large fraction of asthma healthcare expense.

Control of asthma symptoms is typically assessed by a clinician based on a patient's FEV₁, peak expiratory flow, nighttime awakenings, short-acting bronchodilator rescue medication use, or other symptoms that would require an increase in inhaled corticosteroids (ICS) or oral corticosteroids (OCS) dose. Asthma severity is classified by the level of treatment required to control symptoms and exacerbations. The Global Initiative for Asthma (GINA) defines 5 steps of increasing intensity of treatment (GINA steps 1-5). Mild, moderate, and severe asthma correspond to GINA steps 1-2, 3, and 4-5, respectively. Many of the medications developed for asthma are taken by inhalation. Some medications developed for asthma require injection. Current treatment includes monoclonal antibodies, for example, benralizumab, which must be administered subcutaneously every 4 weeks, mepolizumab which must be administered subcutaneously every 4 weeks for 2 doses and 8 weeks thereafter; and reslizumab, which is administered intravenously every 4 weeks.

Accordingly, an oral drug that can reduce or eliminate the symptoms of moderate to severe asthma of the eosinophilic phenotype, such as decreasing the number of exacerbations requiring medical intervention, would be beneficial, particularly a drug that is administered orally and that can be used as a controller medication alone or in combination with the standard of care (such as an inhaled corticosteroid, often referred to as ICS), a long-acting beta-2 agonist (often referred to as a LABA), or a combination thereof. Described herein are methods of using dexpramipexole, or pharmaceutically acceptable salts thereof, in treating moderate to severe asthma of the eosinophilic phenotype.

Not wishing to be bound by theory, inflammation is an important component in the pathogenesis of asthma. IL-5 is the major cytokine responsible for the growth, proliferation, differentiation, recruitment, activation, and survival of eosinophils. Current asthma medications work in a variety of ways. By inhibiting IL-5 signaling, a reduction in the production and survival of eosinophils can be seen. Additional mechanisms include the binding to FcγRIII receptors on immune effectors cells, such as natural killer (NK) cells, leading to apoptosis of eosinophils through antibody-dependent cell-mediated cytotoxicity (ADCC), thus reducing the level of eosinophils. However, a reduction in the levels of eosinophils is not necessarily predictive of clinical success in all eosinophilic diseases. In a previous clinical trial directed to treating eosinophilic chronic rhinosinusitis with nasal polyps with 150 mg dexpramipexole twice daily, it was observed that despite a large and significant change in blood AEC, total polyp score (TPS) was unaffected. Chronic rhinosinusitis with nasal polyps is understood to be an eosinophilic disease and other eosinophil lowering drugs, such as mepolizumab, have shown efficacy. The KNS-760704-CS201 clinical trial was an open-label one arm multi-center 6 month trial of dexpramipexole 150 mg twice daily enrolling 16 subjects with eosinophilic chronic rhinosinusitis with nasal polyps. The co-primary endpoints were change in AEC and change in total polyp score (TPS) from baseline to Month 6, with additional clinical and histologic endpoints assessed. The trial met the first co-primary endpoint, demonstrating 94% lowering of the AEC after 6 months. In contrast to the large and significant change in blood AEC, TPS was unaffected at either the 3 or 6 month timepoints (Baseline 5.29; 3 months 5.50; 6 months 5.42 [mean values]). Other indices of chronic rhinosinusitis disease activity, including SNOT-22 (rhinosinusitis symptoms), sinus CT score, and Sniffin® Sticks olfaction test were also not improved. In the 12 subjects with a history of asthma, the ACQ6 and FEV1 values were unchanged by treatment with dexpramipexole. See also Laidlaw, et al. Dexpramipexole Depletes Blood and Tissue Eosinophils in Nasal Polyps With No Change in Polyp Size, The Laryngoscope, 2019 February; 129(2):E61-E66 (Epub 2018 Oct. 4).

Surprisingly, dexpramipexole further decreases the level of eosinophils not just in peripheral blood but also the tissue of the lungs and nasal passages, which results in superior efficacy in treating moderate to severe asthma of the eosinophilic phenotype, such as by decreasing blood absolute eosinophil count, decreasing the number of exacerbations, increasing FEV1, increasing FVC, increasing peak expiratory flow, improving annualized CompEx event rate, and/or improving subject ACQ-7, ACQ-6 and AQLQ scores. The pathogenesis of moderate to severe asthma also includes the development of mucus plugs in the subject's airways, creating, in effect, a one-way valve, allowing air into the lungs but not efficiently out of the lungs, leading to air trapping and a decrease in forced vital capacity (FVC), and residual volume to total lung capacity ratio (RV/TLC). Dexpramipexole treats these mucus plugs, improves lung capacity, and decreases exacerbations requiring medical intervention. The nasal eosinophil peroxidase (EPX) measurement directly correlates with the level of sputum (tissue) eosinophils and can be utilized as a method to quickly and easily assess the level of tissue involvement and treatment success.

Embodiments are directed to methods for treating moderate to severe asthma of the eosinophilic phenotype in a human subject in need thereof comprising orally administering to the subject a daily dose of about 75 mg to about 300 mg of dexpramipexole, or a pharmaceutically acceptable salt thereof, wherein the subject is already receiving at least two asthma medications, thereby treating the moderate to severe asthma of the eosinophilic phenotype in the subject. In embodiments described herein, the at least two asthma medications are an inhaled corticosteroids (ICS) and a long-acting beta agonist (LABA). In some embodiments, the moderate to severe asthma of the eosinophilic phenotype is moderate asthma. In some embodiments, the moderate to severe asthma of the eosinophilic phenotype is severe asthma.

Preferred embodiments are directed to methods for treating severe asthma of the eosinophilic phenotype in a human subject in need thereof comprising orally administering to the subject a daily dose of about 150 mg to about 300 mg of dexpramipexole, or a pharmaceutically acceptable salt thereof, wherein the subject is already receiving at least two asthma medications, thereby treating the moderate to severe asthma of the eosinophilic phenotype in the subject. In embodiments described herein, the at least two asthma medications is an inhaled corticosteroids (ICS) and a long-acting beta agonist (LABA).

Surprisingly, beta-adrenergic agonist bronchodilator reversibility in subjects taking dexpramipexole is maintained. In other words, dexpramipexole provides a clinical benefit having a different mechanism of action compared with beta agonists, which are medications used by almost all asthmatic patients. Accordingly, the effect of dexpramipexole is additive when taken with other asthma medications.

In some embodiments, the subject has been diagnosed with asthma severity of GINA 3, and diagnosed as having moderate asthma. In some embodiments, the subject has been diagnosed with moderate asthma and requires a low-dose inhaled corticosteroid and a long acting bronchodilator (ICS/LABA) to control symptoms and prevent exacerbations.

In some embodiments, the subject has been diagnosed with asthma severity of GINA 4-5, and diagnosed as having severe asthma. In some embodiments, the subject has been diagnosed with severe asthma and requires a medium-dose inhaled corticosteroid and a long acting bronchodilator (ICS/LABA) to control symptoms and prevent exacerbations. In some embodiments, the subject has been diagnosed with severe asthma and requires a high-dose inhaled corticosteroid and a long acting bronchodilator (ICS/LABA) to control symptoms and prevent exacerbations.

The inhaled corticosteroids (ICS) is selected from the group consisting of beclomethasone, fluticasone, ciclesonide, mometasone, budesonide, flunisolide, and combinations thereof. The inhaled corticosteroids may be low-high dose inhaled corticosteroids, medium-dose ICS, or high-dose ICS. The long-acting beta agonist (LABA) is selected from the group consisting of albuterol sulfate, formoterol fumarate, salmeterol, salmeterol xinafoate, arformoterol tartrate, olodaterol, vilanterol, indacaterol, and combinations thereof. The amount of the inhaled corticosteroid being taken by the subject may be low dose, medium dose, or high dose.

In embodiments described herein, the subject may further be taking an oral corticosteroid (OCS) selected from the group consisting of cortisone acetate, dexamethasone, hydrocortisone, methylprednisolone, prednisolone, prednisone, and combinations thereof. Maintenance oral corticosteroids (OCS) are typically prescribed at up to 20 mg of prednisone per day or equivalent. In embodiments described herein, the subject may further be taking a long-acting muscarinic antagonist (LAMA) selected from the group consisting of umeclidinium, aclidinium, gycopyrronium, glycopyrrolate, tiotropium, and combinations thereof.

In some embodiments, the subject is 18 years of age or older. In some embodiments, the subject is 12 years of age to about 17 years of age. In some embodiments, the subject is about 6 years old to about 11 years old. In some embodiments, the subject is about 2 years old to about 5 years old. In embodiments described herein, the subject is an adult or an adolescent. In embodiments described herein, the adolescents is 12 up to 18 years of age. In embodiments described herein, the subject is younger than 18 years of age. In embodiments described herein, the subject is a child younger than 12 years of age. In embodiments described herein, the subject is 18 years of age or older. In embodiments described herein, the subject is between the ages of 18 to 75 years old. In embodiments described herein, the subject is between the ages of 12 to 75 years old. In embodiments described herein, the subject is 12 years of age or older.

In some embodiments, the moderate to severe asthma of the eosinophilic phenotype in the subject is treated, wherein the subject has a reduction in level of absolute blood eosinophils compared to the level prior to the administration of dexpramipexole.

In some embodiments, the moderate to severe asthma of the eosinophilic phenotype in the subject is treated, wherein the subject shows improvement a measurement selected from the group consisting of forced expiratory volume in 1 second (FEV₁), score on Asthma Control Questionnaire (ACQ), score on Asthma Quality of Life Questionnaire (AQLQ), and combinations thereof.

In some embodiments, the moderate to severe asthma of the eosinophilic phenotype in the subject is treated, wherein the subject has a reduction in a symptom selected from the group consisting of level of nasal eosinophil peroxidase, level of pharyngeal eosinophil peroxidase, level of blood basophils, level of blood eosinophil progenitor population, fractional exhaled nitric oxide, and combinations thereof.

In some embodiments, the moderate to severe asthma of the eosinophilic phenotype in the subject is treated, wherein the subject shows improvement in reduction in frequency of asthma exacerbations.

In some embodiments, the moderate to severe asthma of the eosinophilic phenotype in the subject is treated, wherein the subject shows improvement in a measurement selected from the group consisting of forced expiratory volume in 1 second (FEV1), forced vital capacity (FVC), annualized CompEx event rate, morning peak expiratory flow (PEF), score on Asthma Control Questionnaire (ACQ), score of Asthma Quality of Life Questionnaire (AQLQ), score on St. George's Respiratory Questionnaire, and combinations thereof.

In some embodiments, the moderate to severe asthma of the eosinophilic phenotype in the subject is treated, wherein the subject has a reduction in one or more of symptoms selected from the group consisting of time to first exacerbation, level of nasal eosinophil peroxidase, level of blood eosinophils, and combinations thereof.

In some embodiments, the moderate to severe asthma of the eosinophilic phenotype is treated and the subject has a reduction in one or more of symptoms selected from the group consisting of level of absolute blood eosinophil, level of tissue eosinophils, amount of mucus plugs in the airways, level of nasal eosinophil peroxidase, and combinations thereof.

U.S. Food and Drug Administration (FDA) has developed a clinical resource, BEST (Biomarkers, EndpointS, and other Tools), which defines biomarker used in drug development. A Pharmacodynamic/Response Biomarker is defined as a biomarker that shows that a biological response has occurred in an individual who has been exposed to a medical product, such that one or more expected clinical events linked to that biomarker can be reasonably anticipated. In certain embodiments, a subject's blood absolute eosinophil count can be used as a Pharmacodynamic/Response Biomarker and the subject should continue to be treated with dexpramipexole, if after 12 weeks of therapy the eosinophil count was decreased, even if a clinical response was not yet apparent at week 12. In certain embodiments, a subject's level of nasal eosinophil peroxidase (EPX) can be used as a Pharmacodynamic/Response Biomarker and the subject should continue to be treated with dexpramipexole, if at week 12 the level of EPX had decreased, even if a clinical response was not yet apparent by week 12. These positive Pharmacodynamic/Response Biomarker results suggest that either a patient's blood eosinophil count or nasal EPX level could be used as a tool to manage dexpramipexole in treating the moderate to severe asthma.

In some embodiments, treating moderate to severe asthma of the eosinophilic phenotype by oral administration of a daily dose of about 75 mg to about 300 mg of dexpramipexole results in a reduction of the level of eosinophils to less than 300 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype by oral administration of a daily dose of about 75 mg to about 300 mg of dexpramipexole results in a reduction of the level of eosinophils to less than 250 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype by oral administration of a daily dose of about 75 mg to about 300 mg of dexpramipexole results in a reduction of the level of eosinophils to less than 200 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype by oral administration of a daily dose of about 75 mg to about 300 mg of dexpramipexole results in a reduction of the level of eosinophils to less than 150 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype by oral administration of a daily dose of about 75 mg to about 300 mg of dexpramipexole results in a reduction of the level of eosinophils to less than 100 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype by oral administration of a daily dose of about 75 mg to about 300 mg of dexpramipexole results in a reduction of the level of eosinophils to less than 50 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype by administration of a daily dose of about 75 mg to about 300 mg of dexpramipexole results in a reduction of the level of absolute blood eosinophils by at least about 90%. In certain embodiments, treating moderate to severe asthma of the eosinophilic phenotype by administration of a daily dose of about 75 mg to about 300 mg of dexpramipexole results in a reduction of the level of absolute blood eosinophils by at least about 50%. In certain embodiments, treating moderate to severe asthma of the eosinophilic phenotype by administration of a daily dose of about 300 mg of dexpramipexole results in a reduction of the level of absolute blood eosinophils by about 80%. In certain embodiments, treating moderate to severe asthma of the eosinophilic phenotype by administration of a daily dose of about 150 mg of dexpramipexole results in a reduction of the level of absolute blood eosinophils by about 70%. In certain embodiments, treating moderate to severe asthma of the eosinophilic phenotype by administration of a daily dose of about 75 mg of dexpramipexole results in a reduction of the level of absolute blood eosinophils by about 60%.

In some embodiments, treating severe asthma of the eosinophilic phenotype by administration of a daily dose of about 150 mg to about 300 mg of dexpramipexole results in a reduction of the level of absolute blood eosinophils by at least about 90%. In certain embodiments, treating severe asthma of the eosinophilic phenotype by administration of a daily dose of about 150 mg to about 300 mg of dexpramipexole results in a reduction of the level of absolute blood eosinophils by at least about 50%. In certain embodiments, treating severe asthma of the eosinophilic phenotype by administration of a daily dose of about 300 mg of dexpramipexole results in a reduction of the level of absolute blood eosinophils by about 80%. In certain embodiments, treating severe asthma of the eosinophilic phenotype by administration of a daily dose of about 150 mg of dexpramipexole results in a reduction of the level of absolute blood eosinophils by about 70%. In certain embodiments, treating severe asthma of the eosinophilic phenotype by administration of a daily dose of about 75 mg of dexpramipexole results in a reduction of the level of absolute blood eosinophils by about 60%.

In some embodiments, treating moderate to severe asthma of the eosinophilic phenotype results in a reduction of the level of eosinophils in the tissue. In certain embodiments, treating moderate to severe asthma of the eosinophilic phenotype results in a reduction of the level of eosinophils in tissues including, but not limited to, lung tissue, nasal tissue, bone marrow, and combinations thereof. In certain embodiments, the level of eosinophils in the tissues are reduced by at least 40%, at least 45%, at least 50%, at least 55%, at least 60%, at least 65%, at least 70%, at least 75%, at least 80%, at least 85%, at least 90%, at least 95%, or 100%. In certain embodiments, the levels are reduced to normal. In certain embodiments, the levels are reduced to zero within the level of detection.

In some embodiments, treating moderate to severe asthma of the eosinophilic phenotype results in a reduction in the amount of mucus plugs in the airways. Without wishing to be bound by theory, mucus plugs contribute to chronic airflow obstruction in severe asthma, and eosinophils release eosinophil peroxidase (EPX), which generates reactive oxygen species that oxidize cysteine thiol groups, this leads to cross-linking of the mucin proteins, making the mucus stiffer and promoting mucus plug formation in the bronchi (airways). In embodiments described herein, orally administering a daily dose of about 75 mg to about 300 mg of dexpramipexole, or a pharmaceutically acceptable salt thereof, decreases the level of tissue eosinophils, thus reducing airway eosinophil peroxidase, which in turn, reduces the amount of mucus plugs in the airways, all of which leads to improved lung function and greater exercise tolerance.

In some embodiments, the moderate to severe asthma of the eosinophilic phenotype is treated wherein the concentration of induced sputum eosinophil peroxidase (EPX), nasal eosinophil peroxidase (EPX), or pharyngeal eosinophil peroxidase (EPX) is reduced. Eosinophil peroxidase (EPX) is a granule protein uniquely secreted by eosinophils which can be detected and quantified using an enzyme-linked immunosorbent assay (ELISA). EPX is measured from mucosal swabs or sputum samples. In some embodiments, the subject with moderate to severe asthma of the eosinophilic phenotype is treated and the nasal EPX levels are reduced to normal levels. In embodiments, the nasal and sputum EPX levels following administration of a daily dose of about 75 mg to about 300 mg of dexpramipexole are reduced to level of the nasal and sputum EPX levels in a healthy, non-asthmatic control subject. In embodiments, normal nasal EPX levels are less than about 10 ng/mg of total protein, whereas moderate to severe asthma of the eosinophilic phenotype may be diagnosed as having nasal EPX levels greater than about 10 ng/mg of total protein. In some embodiments, the subject with moderate to severe asthma of the eosinophilic phenotype is treated and the induced sputum EPX levels are reduced to the levels observed in a healthy, non-asthmatic control subject. Normal induced sputum EPX levels may be less than about 200 ng/mL/g recovered sputum, whereas moderate to severe asthma of the eosinophilic phenotype is diagnosed as having induced sputum EPX levels greater than about 200 ng/mL/g recovered sputum. In certain embodiments, treating moderate to severe asthma of the eosinophilic phenotype by administration of a daily dose of about 300 mg of dexpramipexole results in a reduction of the level of nasal EPX by about 89%. In certain embodiments, treating moderate to severe asthma of the eosinophilic phenotype by administration of a daily dose of about 150 mg of dexpramipexole results in a reduction of the level of nasal EPX by about 83%. In certain embodiments, treating moderate to severe asthma of the eosinophilic phenotype by administration of a daily dose of about 75 mg of dexpramipexole results in a reduction of the level of nasal EPX by about 36%.

In some embodiments, the moderate to severe asthma of the eosinophilic phenotype is treated wherein the level of urine eosinophil granule proteins are reduced. In certain embodiments, the eosinophil granule proteins are selected from the group consisting of MBP-1 (Eosinophil Major Basic Protein-1), EDN (Eosinophil-derived Neurotoxin, RNase-2), ECP (Eosinophil Cationic Protein, RNase-3), EPX (Eosinophil Peroxidase), Charcot-Leyden Crystal protein (Gal-10/CLC) and combinations thereof.

In some embodiments, the reduction in blood eosinophils correlates with a reduction in tissue eosinophil biomarkers. In some embodiments, the tissue eosinophil biomarkers are selected from the group consisting of type 2 inflammation associated mediators, including IL-5, IL-13, IL-33, ST2 (IL1RL1), CCL2, CCL3, CCL4, CCL11, CCL17, CCR3, and combinations thereof. Eosinophil development is supported by a variety of cytokines, including Dc-related cytokines, such as GM-CSF, IL-3, and IL-5, which can also be used as biomarkers to assess the reduction in blood eosinophils. In some embodiments, the reduction in blood eosinophils correlates with a reduction in blood eosinophil progenitor populations. Eosinophils are derived from hematopoietic stem cells (HSCs), and both eosinophils and basophils are derived from myeloid progenitor cells.

In some embodiments, the methods of treating moderate to severe asthma of the eosinophilic phenotype in a human subject in need thereof comprising orally administering a daily dose of about 75 mg to about 300 mg of dexpramipexole, wherein the level of blood basophils is reduced.

In some embodiments, the methods of treating severe asthma of the eosinophilic phenotype in a human subject in need thereof comprising orally administering a daily dose of about 150 mg to about 300 mg of dexpramipexole, wherein the level of blood basophils is reduced.

In some embodiments, the moderate to severe asthma of the eosinophilic phenotype is treated and the subject shows improvement in one or more of the measurements selected from the group consisting of reduction in the frequency of asthma exacerbations, reduction the use of oral corticosteroids, reduction in use of inhaled corticosteroids, reduction in use of long-acting beta agonist, improvement the mean forced expiratory volume in 1 second (FEV₁), improvement forced vital capacity (FVC), lowering of residual volume to total lung capacity ratio (RV/TLC), improvement in score on Asthma Control Questionnaire (ACQ), improvement in score on Asthma Control Test (ACT)™, improvement in score of Asthma Quality of Life Questionnaire (AQLQ), or a combination thereof.

In some embodiments, the subject with moderate to severe asthma of the eosinophilic phenotype experienced at least 1 or at least 2 exacerbations over the past 12 months. A severe asthma exacerbation is defined as worsening of asthma requiring the use of systemic corticosteroids and/or hospitalization and/or medical intervention and/or emergency department (ED) visit. In certain embodiments, the medical intervention was corroborated with at least 1 of the following: 1) a decrease in forced expiratory volume in 1 second (FEV₁) by 20% or more from baseline, 2) a decrease in peak expiratory flow rate (PEFR) by 30% or more from baseline on 2 consecutive days, or 3) worsening of symptoms or other clinical signs per physician evaluation of the event. An increase in asthma symptoms requiring oral or intravenous systemic corticosteroid use for at least 3 consecutive days is considered evidence of a severe asthma exacerbation for; for IM corticosteroids (longer acting), a single dose is sufficient. In embodiments, an exacerbation is defined as the worsening of asthma symptoms and lung function requiring use of oral/systemic corticosteroids for at least 3 days. In embodiments, for subjects on maintenance oral corticosteroids, an exacerbation requiring oral/systemic corticosteroids is defined as the use of oral/systemic corticosteroids that is at least double the existing dose for at least 3 days. In embodiments, frequency of exacerbations is defined as the number of exacerbations per a particular defined unit of time, such as 8 weeks, 12 weeks, 24 weeks, 26 weeks, 36 weeks, or 52 weeks. In embodiments described herein, exacerbations requiring the use of a systemic corticosteroid (e.g., OCS) as well as exacerbations resulting in hospitalization or an emergency room visit were each reduced.

In some embodiments, treating moderate to severe asthma of the eosinophilic phenotype by administration of a daily dose of about 75 mg to about 300 mg of dexpramipexole results in a reduction in the number of exacerbations per year. In some embodiments, improvement in exacerbations measured by either fewer exacerbations per unit of time or an extension in the time between the next subsequent exacerbation. In some embodiments, the exacerbation rate is reduced to less than 2 per year, less than 1 per year, or is 0 per year. In some embodiments, the time to first severe asthma exacerbation was extended. In some embodiments, the time to first severe asthma exacerbation did not occur within one year of the previous exacerbation.

In some embodiments, treating severe asthma of the eosinophilic phenotype by administration of a daily dose of about 150 mg to about 300 mg of dexpramipexole results in a reduction in the number of exacerbations per year. In some embodiments, improvement in exacerbations measured by either fewer exacerbations per unit of time or an extension in the time between the next subsequent exacerbation. In some embodiments, the exacerbation rate is reduced to less than 2 per year, less than 1 per year, or is 0 per year. In some embodiments, the time to first severe asthma exacerbation was extended. In some embodiments, the time to first severe asthma exacerbation did not occur within one year of the previous exacerbation.

In some embodiments, treating moderate to severe asthma of the eosinophilic phenotype by administration of a daily dose of about 75 mg to about 300 mg of dexpramipexole results in improvement in the subject's annualized CompEx event rate. Annualized CompEx event rate is a measurement of an extended definition of asthma exacerbations, combining diary-based events with traditionally defined severe exacerbations. Diary events are based on objective measures of deterioration of peak expiratory flow, reliever use, and asthma symptoms assessed morning and evening and night-time awakenings. Deterioration is defined as either reaching a predefined change from baseline (threshold), for at least 2 consecutive days, or deterioration in all variables over at least a 5-day period plus at least one variable reaching a threshold criterion for at least 2 consecutive days. Deterioration of at least two concurrent criteria is needed to fulfill the criteria for a diary event.

In some embodiments, treating severe asthma of the eosinophilic phenotype by administration of a daily dose of about 150 mg to about 300 mg of dexpramipexole results in improvement in the subject's annualized CompEx event rate. Annualized CompEx event rate is a measurement of an extended definition of asthma exacerbations, combining diary-based events with traditionally defined severe exacerbations. Diary events are based on objective measures of deterioration of peak expiratory flow, reliever use, and asthma symptoms assessed morning and evening and night-time awakenings. Deterioration is defined as either reaching a predefined change from baseline (threshold), for at least 2 consecutive days, or deterioration in all variables over at least a 5-day period plus at least one variable reaching a threshold criterion for at least 2 consecutive days. Deterioration of at least two concurrent criteria is needed to fulfill the criteria for a diary event.

In some embodiments, treating moderate to severe asthma of the eosinophilic phenotype by administration of a daily dose of about 75 mg to about 300 mg of dexpramipexole results in a reduction in oral corticosteroid dose. In some embodiments, the oral corticosteroid dose is reduced by about 90% to about 100%, about 75% to about 90%, about 50% to about 75%, or about 10% to about 50%. In some embodiments, the oral corticosteroid dose is reduced to zero. In some embodiments, the frequency of the use of oral corticosteroid is reduced from baseline, such as a 90-100% decrease, a 75-90% decrease, a 50-75% decrease, or at least a 50% reduction. In embodiments described herein, use of oral corticosteroids is reduced to every other day. In embodiments described herein, reductions in oral corticosteroid dose is gradual and performed under the supervision of a physician. In embodiments described herein, reductions in oral corticosteroid dose and frequency occurs while maintaining asthma control.

In some embodiments, treating severe asthma of the eosinophilic phenotype by administration of a daily dose of about 150 mg to about 300 mg of dexpramipexole results in a reduction in oral corticosteroid dose. In some embodiments, the oral corticosteroid dose is reduced by about 90% to about 100%, about 75% to about 90%, about 50% to about 75%, or about 10% to about 50%. In some embodiments, the oral corticosteroid dose is reduced to zero. In some embodiments, the frequency of the use of oral corticosteroid is reduced from baseline, such as a 90-100% decrease, a 75-90% decrease, a 50-75% decrease, or at least a 50% reduction. In embodiments described herein, use of oral corticosteroids is reduced to every other day. In embodiments described herein, reductions in oral corticosteroid dose is gradual and performed under the supervision of a physician. In embodiments described herein, reductions in oral corticosteroid dose and frequency occurs while maintaining asthma control.

In some embodiments, treating moderate to severe asthma of the eosinophilic phenotype by administration of a daily dose of about 75 mg to about 300 mg of dexpramipexole results in a reduction in inhaled corticosteroid dose. In some embodiments, the inhaled corticosteroid dose is reduced by about 90% to about 100%, about 75% to about 90%, about 50% to about 75%, or about 10% to about 50%. In some embodiments, the inhaled corticosteroid dose is reduced to zero. In some embodiments, the frequency of the use of inhaled corticosteroid is reduced from baseline, such as a 90-100% decrease, a 75-90% decrease, a 50-75% decrease, or at least a 50% reduction. In embodiments described herein, the use of inhaled corticosteroids is reduced to less than 3 times per week. In embodiments described herein, reductions in inhaled corticosteroid dose is gradual and performed under the supervision of a physician. In embodiments described herein, reductions in inhaled corticosteroid dose and frequency occurs while maintaining asthma control.

In some embodiments, treating severe asthma of the eosinophilic phenotype by administration of a daily dose of about 150 mg to about 300 mg of dexpramipexole results in a reduction in inhaled corticosteroid dose. In some embodiments, the inhaled corticosteroid dose is reduced by about 90% to about 100%, about 75% to about 90%, about 50% to about 75%, or about 10% to about 50%. In some embodiments, the inhaled corticosteroid dose is reduced to zero. In some embodiments, the frequency of the use of inhaled corticosteroid is reduced from baseline, such as a 90-100% decrease, a 75-90% decrease, a 50-75% decrease, or at least a 50% reduction. In embodiments described herein, the use of inhaled corticosteroids is reduced to less than 3 times per week. In embodiments described herein, reductions in inhaled corticosteroid dose is gradual and performed under the supervision of a physician. In embodiments described herein, reductions in inhaled corticosteroid dose and frequency occurs while maintaining asthma control.

In some embodiments, treating moderate to severe asthma of the eosinophilic phenotype by administration of a daily dose of about 75 mg to about 300 mg of dexpramipexole results in a reduction in long-acting beta agonist dose. In some embodiments, the long-acting beta agonist dose is reduced by about 90% to about 100%, about 75% to about 90%, about 50% to about 75%, or about 10% to about 50%. In some embodiments, the long-acting beta agonist dose is reduced to zero. In some embodiments, the frequency of the use of long-acting beta agonist is reduced from baseline, such as a 90-100% decrease, a 75-90% decrease, a 50-75% decrease, or at least a 50% reduction. In embodiments described herein, the use of long-acting beta agonist is reduced to less than 1 time per day. In embodiments described herein, reductions in long-acting beta agonist dose is gradual and performed under the supervision of a physician. In embodiments described herein, reductions in long-acting beta agonist dose and frequency occurs while maintaining asthma control.

In some embodiments, treating severe asthma of the eosinophilic phenotype by administration of a daily dose of about 150 mg to about 300 mg of dexpramipexole results in a reduction in long-acting beta agonist dose. In some embodiments, the long-acting beta agonist dose is reduced by about 90% to about 100%, about 75% to about 90%, about 50% to about 75%, or about 10% to about 50%. In some embodiments, the long-acting beta agonist dose is reduced to zero. In some embodiments, the frequency of the use of long-acting beta agonist is reduced from baseline, such as a 90-100% decrease, a 75-90% decrease, a 50-75% decrease, or at least a 50% reduction. In embodiments described herein, the use of long-acting beta agonist is reduced to less than 1 time per day. In embodiments described herein, reductions in long-acting beta agonist dose is gradual and performed under the supervision of a physician. In embodiments described herein, reductions in long-acting beta agonist dose and frequency occurs while maintaining asthma control.

Embodiments described herein are directed to methods of treating moderate to severe asthma of the eosinophilic phenotype in a human subject in need thereof comprising orally administering a daily dose of about 75 mg to about 300 mg of dexpramipexole, wherein pulmonary function is improved. Pulmonary function is measured by performing a spirometry, which measures forced expiratory volume in is (FEV₁), the volume of air moved in the first second of exhalation, this is a well-accepted measure of asthma. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

Embodiments described herein are directed to methods of treating severe asthma of the eosinophilic phenotype in a human subject in need thereof comprising orally administering a daily dose of about 150 mg to about 300 mg of dexpramipexole, wherein pulmonary function is improved. Pulmonary function is measured by performing a spirometry, which measures forced expiratory volume in is (FEV₁), the volume of air moved in the first second of exhalation, this is a well-accepted measure of asthma. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

Embodiments described herein are directed to methods of treating moderate to severe asthma of the eosinophilic phenotype in a human subject in need thereof comprising orally administering a daily dose of about 75 mg to about 300 mg of dexpramipexole, wherein FEV₁ is improved. In certain embodiments, the subject had a baseline FEV₁ of about 1.9 L to about 2.2 L at baseline, which is about a 60% reduction from normal/predicted. In some embodiments, the FEV₁ is increased by about 5% to about 20%. More preferably, the FEV₁ is increased by about 10% to about 20%. In some embodiments, the volume of FEV₁ is increased by about 150 ml to about 300 ml. More preferably, the FEV₁ is increased by about 200 ml to about 300 ml.

Embodiments described herein are directed to methods of treating severe asthma of the eosinophilic phenotype in a human subject in need thereof comprising orally administering a daily dose of about 150 mg to about 300 mg of dexpramipexole, wherein FEV₁ is improved. In certain embodiments, the subject had a baseline FEV₁ of about 1.9 L to about 2.2 L at baseline, which is about a 60% reduction from normal/predicted. In some embodiments, the FEV₁ is increased by about 5% to about 20%. More preferably, the FEV₁ is increased by about 10% to about 20%. In some embodiments, the volume of FEV₁ is increased by about 150 ml to about 300 ml. More preferably, the FEV₁ is increased by about 200 ml to about 300 ml.

In some embodiments, treating moderate to severe asthma of the eosinophilic phenotype by administration of a daily dose of about 75 mg to about 300 mg of dexpramipexole results in an increase in the volume of FEV₁ in about 12 weeks, about 8 weeks, about 6 weeks, about 4 weeks, about 3 weeks, or about 2 weeks.

In some embodiments, treating severe asthma of the eosinophilic phenotype by administration of a daily dose of about 150 mg to about 300 mg of dexpramipexole results in an increase in the volume of FEV₁ in about 12 weeks, about 8 weeks, about 6 weeks, about 4 weeks, about 3 weeks, or about 2 weeks.

Embodiments described herein are directed to methods of treating moderate to severe asthma of the eosinophilic phenotype in a human subject in need thereof comprising orally administering a daily dose of about 75 mg to about 300 mg of dexpramipexole, wherein FVC is improved. In certain embodiments, the subject had a baseline FVC of about 3.0 L to about 3.3 L, which is about a 74%-80% reduction from normal/predicted. In some embodiments, the FVC is increased by about 2% to about 12%. More preferably, the FVC is increased by about 8% to about 12%. In some embodiments, the volume of FVC is increased by about 20 ml to about 450 ml. More preferably, the volume of FVC is increased by about 200 ml to about 450 ml.

Embodiments described herein are directed to methods of treating severe asthma of the eosinophilic phenotype in a human subject in need thereof comprising orally administering a daily dose of about 150 mg to about 300 mg of dexpramipexole, wherein FVC is improved. In certain embodiments, the subject had a baseline FVC of about 3.0 L to about 3.3 L, which is about a 74%-80% reduction from normal/predicted. In some embodiments, the FVC is increased by about 2% to about 12%. More preferably, the FVC is increased by about 8% to about 12%. In some embodiments, the volume of FVC is increased by about 20 ml to about 450 ml. More preferably, the volume of FVC is increased by about 200 ml to about 450 ml.

In some embodiments, treating moderate to severe asthma of the eosinophilic phenotype by administration of a daily dose of about 75 mg to about 300 mg of dexpramipexole results in an increase in the volume of FVC in about 12 weeks, about 8 weeks, about 6 weeks, about 4 weeks, about 3 weeks, or about 2 weeks.

In some embodiments, treating severe asthma of the eosinophilic phenotype by administration of a daily dose of about 150 mg to about 300 mg of dexpramipexole results in an increase in the volume of FVC in about 12 weeks, about 8 weeks, about 6 weeks, about 4 weeks, about 3 weeks, or about 2 weeks.

In some embodiments, treating moderate to severe asthma of the eosinophilic phenotype by administration of a daily dose of about 75 mg to about 300 mg of dexpramipexole results in an increase in morning peak expiratory flow (PEF). Peak flow zones are areas of measurement on a peak flow meter. The goal of the peak flow zones is to show early symptoms of uncontrolled asthma. The green zone is 80% to 100% of the subject's highest peak flow reading. The yellow zone is 50% to 80% of the subject's highest peak flow reading. Measurements in this zone are a sign that large airways are starting to narrow and subject may start to have mild symptoms, such as coughing, feeling tired, feeling short of breath, or chest tightening. The red zone is less than 50% of the subject's highest peak flow reading. Readings in this zone mean severe narrowing of large airways, which is a medical emergency. In some embodiments, treating moderate to severe asthma of the eosinophilic phenotype by administration of a daily dose of about 75 mg to about 300 mg of dexpramipexole results in the subject's ability to remain in the green zone when measuring morning peak expiratory flow.

In some embodiments, treating severe asthma of the eosinophilic phenotype by administration of a daily dose of about 150 mg to about 300 mg of dexpramipexole results in an increase in morning peak expiratory flow (PEF). Peak flow zones are areas of measurement on a peak flow meter. The goal of the peak flow zones is to show early symptoms of uncontrolled asthma. The green zone is 80% to 100% of the subject's highest peak flow reading. The yellow zone is 50% to 80% of the subject's highest peak flow reading. Measurements in this zone are a sign that large airways are starting to narrow and subject may start to have mild symptoms, such as coughing, feeling tired, feeling short of breath, or chest tightening. The red zone is less than 50% of the subject's highest peak flow reading. Readings in this zone mean severe narrowing of large airways, which is a medical emergency. In some embodiments, treating severe asthma of the eosinophilic phenotype by administration of a daily dose of about 150 mg to about 300 mg of dexpramipexole results in the subject's ability to remain in the green zone when measuring morning peak expiratory flow.

Airway inflammation can also be assessed using the FeNO test, fractional concentration of exhaled nitric oxide, wherein the level of nitric oxide is measured in parts per billion (ppb) in the air you slowly exhale. Higher than normal levels of exhaled nitric oxide generally means the airways are inflamed, a sign of asthma. Levels less than about 20 ppb in children and less than about 25 ppb in adults are considered normal. Greater than about 35 ppb in children and 50 ppb in adults are considered to be abnormal.

Embodiments described herein are directed to methods of treating moderate to severe asthma of the eosinophilic phenotype in a human subject in need thereof comprising orally administering a daily dose of about 75 mg to about 300 mg of dexpramipexole, wherein moderate to severe asthma of the eosinophilic phenotype is controlled. The Asthma Control Questionnaire (ACQ), and/or the Asthma Control Test (ACT)™ can be used to assess whether asthma is being controlled. In certain embodiments, the subject had a baseline score of about 1.9 to about 2.3 on the ACQ and about 4.5 to about 5.4 on the AQLQ.

Embodiments described herein are directed to methods of treating severe asthma of the eosinophilic phenotype in a human subject in need thereof comprising orally administering a daily dose of about 150 mg to about 300 mg of dexpramipexole, wherein moderate to severe asthma of the eosinophilic phenotype is controlled. The Asthma Control Questionnaire (ACQ), and/or the Asthma Control Test (ACT)™ can be used to assess whether asthma is being controlled. In certain embodiments, the subject had a baseline score of about 1.9 to about 2.3 on the ACQ and about 4.5 to about 5.4 on the AQLQ.

The ACQ cutoff for well-controlled asthma is a score of about 0.75, and the ACQ cutoff for uncontrolled asthma is a score of about ≥1.5. An ACQ score change of ≥0.5 is considered clinically significant. In embodiments described herein, the subject has a change in ACQ score of at least 0.5 points. In embodiments described herein, the subject has an improvement in score on ACQ and the score is less than or equal to 0.75.

Using the Asthma Control Test (ACT)™, a score of less than 19 indicates the individual's asthma may not be well controlled and a score of less than 15 indicates the individual's asthma may be very poorly controlled. In embodiments described herein, the subject has a change in ACT score of at least 0.5 points. In embodiments described herein, the subject has an improvement in score on Asthma Control Test (ACT)™ and the score is greater than 19.

The AQLQ is a 32-item asthma specific questionnaire designed to measure functional impairments that are most important to patients with asthma. The 32 questions in the AQLQ are divided into four domains; activity limitations, symptoms, emotional function, and environmental stimuli. Individual questions are equally weighted. The overall AQLQ score is the mean of the responses to each of the 32 questions and ranges from 1 to 7. A score 7.0 indicates that the patient has no impairments due to asthma and score 1.0 indicates severe impairment. Embodiments described herein are directed to methods of treating moderate to severe asthma of the eosinophilic phenotype in a human subject in need thereof comprising orally administering a daily dose of about 75 mg to about 300 mg of dexpramipexole, wherein the subject's quality of life is improved. Embodiments described herein are directed to methods of treating severe asthma of the eosinophilic phenotype in a human subject in need thereof comprising orally administering a daily dose of about 150 mg to about 300 mg of dexpramipexole, wherein the subject's quality of life is improved. The Asthma Quality of Life Questionnaire(AQLQ) can be used to assess an individual's quality of life, scores range from 1-7, with higher scores indicating better quality of life. In embodiments described herein, the subject has a change in AQLQ score of at least 0.5 points. In embodiments described herein, the subject has an improvement in score on Asthma Quality of Life Questionnaire (AQLQ) and the score is greater than 3.5.

In embodiments described herein, the subject has an improvement in score on the St. George's Respiratory Questionnaire.

In some embodiments, treating moderate to severe asthma of the eosinophilic phenotype by administration of a daily dose of about 75 mg to about 300 mg of dexpramipexole results in improving a subject's overall morbidity. In some embodiments, treating moderate to severe asthma of the eosinophilic phenotype by administration of a daily dose of about 75 mg to about 300 mg of dexpramipexole results in improving a subject's overall mortality risk.

In some embodiments, treating severe asthma of the eosinophilic phenotype by administration of a daily dose of about 150 mg to about 300 mg of dexpramipexole results in improving a subject's overall morbidity. In some embodiments, treating severe asthma of the eosinophilic phenotype by administration of a daily dose of about 150 mg to about 300 mg of dexpramipexole results in improving a subject's overall mortality risk.

Hematologic Responders

Embodiments are directed to methods for treating moderate to severe asthma of the eosinophilic phenotype in a hematologic responder human subject comprising orally administering to the hematologic responder human subject a daily dose of about 75 mg to about 300 mg of dexpramipexole, or a pharmaceutically acceptable salt thereof, wherein the hematologic responder human subject is already receiving at least two asthma medications, thereby treating the moderate to severe asthma of the eosinophilic phenotype in the hematologic responder human subject, and improving the FEV₁ by at least 150 ml or at least 5%. In certain embodiments, the hematologic responder human subject experiences the improvement in FEV₁ by about week 4 of treatment with dexpramipexole.

In some embodiments, the hematologic responder human subject has a reduction in absolute eosinophil count to less than 100 cell/μl.

The inhaled corticosteroids (ICS) is selected from the group consisting of beclomethasone, fluticasone, ciclesonide, mometasone, budesonide, flunisolide, and combinations thereof. The inhaled corticosteroids may be medium-high dose inhaled corticosteroids or high-dose ICS. The long-acting beta agonist (LABA) is selected from the group consisting of albuterol sulfate, formoterol fumarate, salmeterol, salmeterol xinafoate, arformoterol tartrate, olodaterol, vilanterol, indacaterol, and combinations thereof. The amount of the inhaled corticosteroid being taken by the subject may be low dose, medium dose or high dose.

In embodiments described herein, the hematologic responder human subject may further be taking an oral corticosteroid (OCS) selected from the group consisting of cortisone acetate, dexamethasone, hydrocortisone, methylprednisolone, prednisolone, prednisone, and combinations thereof. Maintenance oral corticosteroids (OCS) (up to 10 mg of prednisone per day or equivalent). In embodiments described herein, the hematologic responder human subject may further be taking a long-acting muscarinic antagonist (LAMA) selected from the group consisting of umeclidinium, aclidinium, gycopyrronium, glycopyrrolate, tiotropium, and combinations thereof.

In some embodiments, the hematologic responder human subject is 18 years of age or older. In some embodiments, the hematologic responder human subject is 12 years of age to about 17 years of age. In some embodiments, the hematologic responder human subject is about 6 years old to about 11 years old. In some embodiments, the hematologic responder human subject is about 2 years old to about 5 years old. In embodiments described herein, the hematologic responder human subject is an adult or an adolescent. In embodiments described herein, the adolescents is 12 up to 18 years of age. In embodiments described herein, the hematologic responder human subject is younger than 18 years of age. In embodiments described herein, the hematologic responder human subject is a child younger than 12 years of age. In embodiments described herein, the hematologic responder human subject is 18 years of age or older. In embodiments described herein, the hematologic responder human subject is between the ages of 18 to 75 years old. In embodiments described herein, the hematologic responder human subject is between the ages of 12 to 75 years old. In embodiments described herein, the hematologic responder human subject is 12 years of age or older.

In some embodiments, the moderate to severe asthma of the eosinophilic phenotype in the hematologic responder human subject is treated, wherein the hematologic responder human subject shows improvement a measurement selected from the group consisting of forced expiratory volume in 1 second (FEV₁), score on Asthma Control Questionnaire (ACQ), score on Asthma Quality of Life Questionnaire (AQLQ), and combinations thereof.

In some embodiments, the moderate to severe asthma of the eosinophilic phenotype in the hematologic responder human subject is treated, wherein the subject has a reduction in a symptom selected from the group consisting of level of nasal eosinophil peroxidase, level of pharyngeal eosinophil peroxidase, level of blood basophils, level of blood eosinophil progenitor population, fractional exhaled nitric oxide, and combinations thereof.

In some embodiments, the moderate to severe asthma of the eosinophilic phenotype in the hematologic responder human subject is treated, wherein the subject shows improvement in reduction in frequency of asthma exacerbations.

In some embodiments, the moderate to severe asthma of the eosinophilic phenotype in the hematologic responder human subject is treated, wherein the subject shows improvement in a measurement selected from the group consisting of forced expiratory volume in 1 second (FEV₁), forced vital capacity (FVC), annualized CompEx event rate, morning peak expiratory flow (PEF), score on Asthma Control Questionnaire (ACQ), score of Asthma Quality of Life Questionnaire (AQLQ), score on St. George's Respiratory Questionnaire, and combinations thereof.

In some embodiments, the moderate to severe asthma of the eosinophilic phenotype in the hematologic responder human subject is treated, wherein the subject has a reduction in one or more of symptoms selected from the group consisting of time to first exacerbation, level of nasal eosinophil peroxidase, level of blood eosinophils, and combinations thereof.

In some embodiments, the moderate to severe asthma of the eosinophilic phenotype is treated and the hematologic responder human subject has a reduction in one or more of symptom selected from the group consisting of level of absolute blood eosinophil, level of tissue eosinophils, amount of mucus plugs in the airways, level of nasal eosinophil peroxidase, and combinations thereof.

In some embodiments, treating moderate to severe asthma of the eosinophilic phenotype in a hematologic responder human subject by oral administration of a daily dose of about 75 mg to about 300 mg of dexpramipexole results in a reduction of the level of eosinophils to less than 300 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype in a hematologic responder human subject by oral administration of a daily dose of about 75 mg to about 300 mg of dexpramipexole results in a reduction of the level of eosinophils to less than 250 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype in a hematologic responder human subject by oral administration of a daily dose of about 75 mg to about 300 mg of dexpramipexole results in a reduction of the level of eosinophils to less than 200 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype in a hematologic responder human subject by oral administration of a daily dose of about 75 mg to about 300 mg of dexpramipexole results in a reduction of the level of eosinophils to less than 150 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype in a hematologic responder human subject by oral administration of a daily dose of about 75 mg to about 300 mg of dexpramipexole results in a reduction of the level of eosinophils to less than 100 cells per microliter in the peripheral blood by a period of time selected from the group consisting of 12 weeks, 8 weeks, 6 weeks, 4 weeks, 3 weeks, or 2 weeks.

In some embodiments, treating moderate to severe asthma of the eosinophilic phenotype in a hematologic responder human subject by administration of a daily dose of about 75 mg to about 300 mg of dexpramipexole results in a reduction of the level of absolute blood eosinophils by at least about 90%. In certain embodiments, treating moderate to severe asthma of the eosinophilic phenotype in a hematologic responder human subject by administration of a daily dose of about 75 mg to about 300 mg of dexpramipexole results in a reduction of the level of absolute blood eosinophils by at least about 50%. In certain embodiments, treating moderate to severe asthma of the eosinophilic phenotype in a hematologic responder human subject by administration of a daily dose of about 300 mg of dexpramipexole results in a reduction of the level of absolute blood eosinophils by about 80%. In certain embodiments, treating moderate to severe asthma of the eosinophilic phenotype in a hematologic responder human subject by administration of a daily dose of about 150 mg of dexpramipexole results in a reduction of the level of absolute blood eosinophils by about 70%. In certain embodiments, treating moderate to severe asthma of the eosinophilic phenotype in a hematologic responder human subject by administration of a daily dose of about 75 mg of dexpramipexole results in a reduction of the level of absolute blood eosinophils by about 60%.

In some embodiments, treating severe asthma of the eosinophilic phenotype in a hematologic responder human subject by administration of a daily dose of about 150 mg to about 300 mg of dexpramipexole results in a reduction of the level of absolute blood eosinophils by at least about 90%. In certain embodiments, treating severe asthma of the eosinophilic phenotype in a hematologic responder human subject by administration of a daily dose of about 150 mg to about 300 mg of dexpramipexole results in a reduction of the level of absolute blood eosinophils by at least about 50%. In certain embodiments, treating severe asthma of the eosinophilic phenotype in a hematologic responder human subject by administration of a daily dose of about 300 mg of dexpramipexole results in a reduction of the level of absolute blood eosinophils by about 80%. In certain embodiments, treating severe asthma of the eosinophilic phenotype in a hematologic responder human subject by administration of a daily dose of about 150 mg of dexpramipexole results in a reduction of the level of absolute blood eosinophils by about 70%. In certain embodiments, treating severe asthma of the eosinophilic phenotype in a hematologic responder human subject by administration of a daily dose of about 75 mg of dexpramipexole results in a reduction of the level of absolute blood eosinophils by about 60%.

In some embodiments, treating moderate to severe asthma of the eosinophilic phenotype in a hematologic responder human subject results in a reduction of the level of eosinophils in the tissue. In certain embodiments, treating moderate to severe asthma of the eosinophilic phenotype in a hematologic responder human subject results in a reduction of the level of eosinophils in tissues including, but not limited to, lung tissue, nasal tissue, bone marrow, and combinations thereof. In certain embodiments, the level of eosinophils in the tissues are reduced by at least 40%, at least 45%, at least 50%, at least 55%, at least 60%, at least 65%, at least 70%, at least 75%, at least 80%, at least 85%, at least 90%, at least 95%, or 100%. In certain embodiments, the levels are reduced to normal. In certain embodiments, the levels are reduced to zero within the level of detection.

In some embodiments, treating moderate to severe asthma of the eosinophilic phenotype in a hematologic responder human subject results in a reduction in the amount of mucus plugs in the airways. Without wishing to be bound by theory, mucus plugs contribute to chronic airflow obstruction in severe asthma, and eosinophils release eosinophil peroxidase (EPX), which generates reactive oxygen species that oxidize cysteine thiol groups, this leads to cross-linking of the mucin proteins, making the mucus stiffer and promoting mucus plug formation in the bronchi (airways). In embodiments described herein, orally administering a daily dose of about 75 mg to about 300 mg of dexpramipexole, or a pharmaceutically acceptable salt thereof, decreases the level of tissue eosinophils, thus reducing airway eosinophil peroxidase, which in turn, reduces the amount of mucus plugs in the airways, all of which leads to improved lung function and greater exercise tolerance.

In some embodiments, the moderate to severe asthma of the eosinophilic phenotype in a hematologic responder human subject is treated wherein the concentration of induced sputum eosinophil peroxidase (EPX), nasal eosinophil peroxidase (EPX), or pharyngeal eosinophil peroxidase (EPX) is reduced. Eosinophil peroxidase (EPX) is a granule protein uniquely secreted by eosinophils which can be detected and quantified using an enzyme-linked immunosorbent assay (ELISA). EPX is measured from mucosal swabs or sputum samples. In some embodiments, the moderate to severe asthma of the eosinophilic phenotype in a hematologic responder human subject is treated and the nasal EPX levels are reduced to normal levels. In embodiments, the nasal and sputum EPX levels following administration of a daily dose of about 75 mg to about 300 mg of dexpramipexole are reduced to level of the nasal and sputum EPX levels in a healthy, non-asthmatic control subject. In embodiments, normal nasal EPX levels are less than about 10 ng/mg of total protein, whereas moderate to severe asthma of the eosinophilic phenotype identified as a hematologic responder human subject may be diagnosed as having nasal EPX levels greater than about 10 ng/mg of total protein. In some embodiments, the moderate to severe asthma of the eosinophilic phenotype in a hematologic responder human subject is treated and the induced sputum EPX levels are reduced to the levels observed in a healthy, non-asthmatic control subject. Normal induced sputum EPX levels may be less than about 200 ng/mL/g recovered sputum, whereas moderate to severe asthma of the eosinophilic phenotype identified as a hematologic responder human subject is diagnosed as having induced sputum EPX levels greater than about 200 ng/mL/g recovered sputum. In certain embodiments, treating moderate to severe asthma of the eosinophilic phenotype in a hematologic responder human subject by administration of a daily dose of about 300 mg of dexpramipexole results in a reduction of the level of nasal EPX by about 89%. In certain embodiments, treating moderate to severe asthma of the eosinophilic phenotype in a hematologic responder human subject by administration of a daily dose of about 150 mg of dexpramipexole results in a reduction of the level of nasal EPX by about 83%. In certain embodiments, treating moderate to severe asthma of the eosinophilic phenotype in a hematologic responder human subject by administration of a daily dose of about 75 mg of dexpramipexole results in a reduction of the level of nasal EPX by about 36%.

In some embodiments, the moderate to severe asthma of the eosinophilic phenotype in a hematologic responder human subject is treated wherein the level of urine eosinophil granule proteins are reduced. In certain embodiments, the eosinophil granule proteins are selected from the group consisting of MBP-1 (Eosinophil Major Basic Protein-1), EDN (Eosinophil-derived Neurotoxin, RNase-2), ECP (Eosinophil Cationic Protein, RNase-3), EPX (Eosinophil Peroxidase), Charcot-Leyden Crystal protein (Gal-10/CLC) and combinations thereof.

In some embodiments, the reduction in blood eosinophils correlates with a reduction in tissue eosinophil biomarkers. In some embodiments, the tissue eosinophil biomarkers are selected from the group consisting of type 2 inflammation associated mediators, including IL-5, IL-13, IL-33, ST2 (IL1RL1), CCL2, CCL3, CCL4, CCL11, CCL17, CCR3, and combinations thereof. Eosinophil development is supported by a variety of cytokines, including Pc-related cytokines, such as GM-CSF, IL-3, and IL-5, which can also be used as biomarkers to assess the reduction in blood eosinophils. In some embodiments, the reduction in blood eosinophils correlates with a reduction in blood eosinophil progenitor populations. Eosinophils are derived from hematopoietic stem cells (HSCs), and both eosinophils and basophils are derived from myeloid progenitor cells.

In some embodiments, the methods of treating moderate to severe asthma of the eosinophilic phenotype in a hematologic responder human subject comprising orally administering a daily dose of about 75 mg to about 300 mg of dexpramipexole, wherein the level of blood basophils is reduced.

In some embodiments, the moderate to severe asthma of the eosinophilic phenotype in a hematologic responder human subject is treated and the hematologic responder human subject shows improvement in one or more of the measurements selected from the group consisting of reduction in the frequency of asthma exacerbations, reduction the use of oral corticosteroids, reduction in use of inhaled corticosteroids, reduction in use of long-acting beta agonist, improvement the mean forced expiratory volume in 1 second (FEV₁), improvement forced vital capacity (FVC), improvement in score on Asthma Control Questionnaire (ACQ), improvement in score on Asthma Control Test (ACT)™, improvement in score of Asthma Quality of Life Questionnaire (AQLQ), or a combination thereof.

In some embodiments, the subject with moderate to severe asthma of the eosinophilic phenotype in a hematologic responder human subject experienced at least 1 or at least 2 exacerbations over the past 12 months. A severe asthma exacerbation is defined as worsening of asthma requiring the use of systemic corticosteroids and/or hospitalization and/or medical intervention and/or emergency department (ED) visit. In certain embodiments, the medical intervention was corroborated with at least 1 of the following: 1) a decrease in forced expiratory volume in 1 second (FEV₁) by 20% or more from baseline, 2) a decrease in peak expiratory flow rate (PEFR) by 30% or more from baseline on 2 consecutive days, or 3) worsening of symptoms or other clinical signs per physician evaluation of the event. Systemic corticosteroid use is considered evidence of a severe asthma exacerbation for intravenous or oral routes if taken for at least 3 consecutive days; for the IM route, a single dose is sufficient. In embodiments, an exacerbation is defined as the worsening of asthma symptoms and lung function requiring use of oral/systemic corticosteroids for at least 3 days. In embodiments, for subjects on maintenance oral corticosteroids, an exacerbation requiring oral/systemic corticosteroids is defined as the use of oral/systemic corticosteroids that is at least double the existing dose for at least 3 days. In embodiments, frequency of exacerbations is defined as the number of exacerbations per a particular defined unit of time, such as 8 weeks, 12 weeks, 24 weeks, 26 weeks, 36 weeks, or 52 weeks. In embodiments described herein, exacerbations requiring the use of a systemic corticosteroid (e.g., OCS) as well as exacerbations resulting in hospitalization or an emergency room visit were each reduced. In some embodiments, treating moderate to severe asthma of the eosinophilic phenotype in a hematologic responder human subject by administration of a daily dose of about 75 mg to about 300 mg of dexpramipexole results in a reduction in the number of exacerbations per year. In some embodiments, improvement in exacerbations measured by either fewer exacerbations per unit of time or an extension in the time between the next subsequent exacerbation. In some embodiments, the exacerbation rate is reduced to less than 2 per year, less than 1 per year, or is 0 per year. In some embodiments, the time to first severe asthma exacerbation was extended. In some embodiments, the time to first severe asthma exacerbation did not occur within one year of the previous exacerbation.

In some embodiments, treating moderate to severe asthma of the eosinophilic phenotype in a hematologic responder human subject by administration of a daily dose of about 75 mg to about 300 mg of dexpramipexole results in improvement in the subject's annualized CompEx event rate. Annualized CompEx event rate is a measurement of an extended definition of asthma exacerbations, combining diary-based events with traditionally defined severe exacerbations. Diary events are based on objective measures of deterioration of peak expiratory flow, reliever use, and asthma symptoms assessed morning and evening and night-time awakenings. Deterioration is defined as either reaching a predefined change from baseline (threshold), for at least 2 consecutive days, or deterioration in all variables over at least a 5-day period plus at least one variable reaching a threshold criterion for at least 2 consecutive days. Deterioration of at least two concurrent criteria is needed to fulfill the criteria for a diary event.

In some embodiments, treating moderate to severe asthma of the eosinophilic phenotype in a hematologic responder human subject by administration of a daily dose of about 75 mg to about 300 mg of dexpramipexole results in a reduction in oral corticosteroid dose. In some embodiments, the oral corticosteroid dose is reduced by about 90% to about 100%, about 75% to about 90%, about 50% to about 75%, or about 10% to about 50%. In some embodiments, the oral corticosteroid dose is reduced to zero. In some embodiments, the frequency of the use of oral corticosteroid is reduced from baseline, such as a 90-100% decrease, a 75-90% decrease, a 50-75% decrease, or at least a 50% reduction. In embodiments described herein, use of oral corticosteroids is reduced to every other day. In embodiments described herein, reductions in oral corticosteroid dose is gradual and performed under the supervision of a physician. In embodiments described herein, reductions in oral corticosteroid dose and frequency occurs while maintaining asthma control.

In some embodiments, treating moderate to severe asthma of the eosinophilic phenotype in a hematologic responder human subject by administration of a daily dose of about 75 mg to about 300 mg of dexpramipexole results in a reduction in inhaled corticosteroid dose. In some embodiments, the inhaled corticosteroid dose is reduced by about 90% to about 100%, about 75% to about 90%, about 50% to about 75%, or about 10% to about 50%. In some embodiments, the inhaled corticosteroid dose is reduced to zero. In some embodiments, the frequency of the use of inhaled corticosteroid is reduced from baseline, such as a 90-100% decrease, a 75-90% decrease, a 50-75% decrease, or at least a 50% reduction. In embodiments described herein, the use of inhaled corticosteroids is reduced to less than 3 times per week. In embodiments described herein, reductions in inhaled corticosteroid dose is gradual and performed under the supervision of a physician. In embodiments described herein, reductions in inhaled corticosteroid dose and frequency occurs while maintaining asthma control.

In some embodiments, treating moderate to severe asthma of the eosinophilic phenotype in a hematologic responder human subject by administration of a daily dose of about 75 mg to about 300 mg of dexpramipexole results in a reduction in long-acting beta agonist dose. In some embodiments, the long-acting beta agonist dose is reduced by about 90% to about 100%, about 75% to about 90%, about 50% to about 75%, or about 10% to about 50%. In some embodiments, the long-acting beta agonist dose is reduced to zero. In some embodiments, the frequency of the use of long-acting beta agonist is reduced from baseline, such as a 90-100% decrease, a 75-90% decrease, a 50-75% decrease, or at least a 50% reduction. In embodiments described herein, the use of long-acting beta agonist is reduced to less than 1 time per day. In embodiments described herein, reductions in long-acting beta agonist dose is gradual and performed under the supervision of a physician. In embodiments described herein, reductions in long-acting beta agonist dose and frequency occurs while maintaining asthma control.

Embodiments described herein are directed to methods of treating moderate to severe asthma of the eosinophilic phenotype in a hematologic responder human subject comprising orally administering a daily dose of about 75 mg to about 300 mg of dexpramipexole, wherein pulmonary function is improved. Pulmonary function is measured by performing a spirometry, which measures forced expiratory volume in 1s (FEV₁), the volume of air moved in the first second of exhalation, this is a well-accepted measure of asthma. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

Embodiments described herein are directed to methods of treating moderate to severe asthma of the eosinophilic phenotype in a hematologic responder human subject comprising orally administering a daily dose of about 75 mg to about 300 mg of dexpramipexole, wherein FEV₁ is improved. In certain embodiments, the hematologic responder human subject had a baseline FEV₁ of about 1.9 L to about 2.2 L at baseline, which is about a 60% reduction from normal/predicted. In some embodiments, the FEV₁ is increased by about 5% to about 20%. More preferably, the FEV₁ is increased by about 10% to about 20%. In some embodiments, the volume of FEV₁ is increased by about 150 ml to about 300 ml. More preferably, the FEV₁ is increased by about 200 ml to about 300 ml.

In some embodiments, treating moderate to severe asthma of the eosinophilic phenotype in a hematologic responder human subject by administration of a daily dose of about 75 mg to about 300 mg of dexpramipexole results in an increase in the volume of FEV₁ in about 12 weeks, about 8 weeks, about 6 weeks, about 4 weeks, about 3 weeks, or about 2 weeks.

Embodiments described herein are directed to methods of treating moderate to severe asthma of the eosinophilic phenotype in a hematologic responder human subject comprising orally administering a daily dose of about 75 mg to about 300 mg of dexpramipexole, wherein FVC is improved. In certain embodiments, the hematologic responder human subject had a baseline FVC of about 3.0 L to about 3.3 L, which is about a 74%-80% reduction from normal/predicted. In some embodiments, the FVC is increased by about 2% to about 12%. More preferably, the FVC is increased by about 8% to about 12%. In some embodiments, the volume of FVC is increased by about 20 ml to about 450 ml. More preferably, the volume of FVC is increased by about 200 ml to about 450 ml.

In some embodiments, treating moderate to severe asthma of the eosinophilic phenotype in a hematologic responder human subject by administration of a daily dose of about 75 mg to about 300 mg of dexpramipexole results in an increase in the volume of FVC in about 12 weeks, about 8 weeks, about 6 weeks, about 4 weeks, about 3 weeks, or about 2 weeks.

In some embodiments, treating moderate to severe asthma of the eosinophilic phenotype in a hematologic responder human subject by administration of a daily dose of about 75 mg to about 300 mg of dexpramipexole results in an increase in morning peak expiratory flow (PEF). Peak flow zones are areas of measurement on a peak flow meter. The goal of the peak flow zones is to show early symptoms of uncontrolled asthma. The green zone is 80% to 100% of the subject's highest peak flow reading. The yellow zone is 50% to 80% of the subject's highest peak flow reading. Measurements in this zone are a sign that large airways are starting to narrow and subject may start to have mild symptoms, such as coughing, feeling tired, feeling short of breath, or chest tightening. The red zone is less than 50% of the subject's highest peak flow reading. Readings in this zone mean severe narrowing of large airways, which is a medical emergency. In some embodiments, treating moderate to severe asthma of the eosinophilic phenotype in a hematologic responder human subject by administration of a daily dose of about 75 mg to about 300 mg of dexpramipexole results in the subject's ability to remain in the green zone when measuring morning peak expiratory flow.

Airway inflammation can also be assessed using the FeNO test, fractional concentration of exhaled nitric oxide, wherein the level of nitric oxide is measured in parts per billion (ppb) in the air you slowly exhale. Higher than normal levels of exhaled nitric oxide generally means the airways are inflamed, a sign of asthma. Levels less than about 20 ppb in children and less than about 25 ppb in adults are considered normal. Greater than about 35 ppb in children and 50 ppb in adults are considered to be abnormal.

Embodiments described herein are directed to methods of treating moderate to severe asthma of the eosinophilic phenotype in a hematologic responder human subject comprising orally administering a daily dose of about 75 mg to about 300 mg of dexpramipexole, wherein moderate to severe asthma of the eosinophilic phenotype in a hematologic responder human subject is controlled. The Asthma Control Questionnaire (ACQ), and/or the Asthma Control Test (ACT)™ can be used to assess whether asthma is being controlled. In certain embodiments, the hematologic responder human subject had a baseline score of about 1.9 to about 2.3 on the ACQ and about 4.5 to about 5.4 on the AQLQ.

The ACQ cutoff for controlled asthma is a score of about 0.75, and the ACQ cutoff for uncontrolled asthma is a score of about 1.5. An ACQ score change of ≥0.5 is considered clinically significant. In embodiments described herein, the hematologic responder human subject has a change in ACQ score of at least 0.5 points. In embodiments described herein, the hematologic responder human subject has an improvement in score on ACQ and the score is less than or equal to 0.75.

Using the Asthma Control Test (ACT)™, a score of less than 19 indicates the individual's asthma may not be well controlled and a score of less than 15 indicates the individual's asthma may be very poorly controlled. In embodiments described herein, the hematologic responder human subject has a change in ACT score of at least 0.5 points. In embodiments described herein, the hematologic responder human subject has an improvement in score on Asthma Control Test (ACT)™ and the score is greater than 19.

The AQLQ is a 32-item asthma specific questionnaire designed to measure functional impairments that are most important to patients with asthma. The 32 questions in the AQLQ are divided into four domains; activity limitations, symptoms, emotional function, and environmental stimuli. Individual questions are equally weighted. The overall AQLQ score is the mean of the responses to each of the 32 questions and ranges from 1 to 7. A score 7.0 indicates that the patient has no impairments due to asthma and score 1.0 indicates severe impairment. Embodiments described herein are directed to methods of treating moderate to severe asthma of the eosinophilic phenotype in a hematologic responder human subject comprising orally administering a daily dose of about 75 mg to about 300 mg of dexpramipexole, wherein the subject's quality of life is improved. The Asthma Quality of Life Questionnaire (AQLQ) can be used to assess an individual's quality of life, scores range from 1-7, with higher scores indicating better quality of life. In embodiments described herein, the hematologic responder human subject has a change in AQLQ score of at least 0.5 points. In embodiments described herein, the hematologic responder human subject has an improvement in score on Asthma Quality of Life Questionnaire (AQLQ) and the score is greater than 3.5.

In embodiments described herein, the hematologic responder human subject has an improvement in score on the St. George's Respiratory Questionnaire.

In some embodiments, treating moderate to severe asthma of the eosinophilic phenotype in a hematologic responder human subject by administration of a daily dose of about 75 mg to about 300 mg of dexpramipexole results in improving a subject's overall morbidity. In some embodiments, treating moderate to severe asthma of the eosinophilic phenotype in a hematologic responder human subject by administration of a daily dose of about 75 mg to about 300 mg of dexpramipexole results in improving a subject's overall mortality risk.

Not-Well Controlled Mild, Moderate, and Severe Asthma

Embodiments are directed to methods for treating not-well controlled mild, moderate, or severe asthma of the eosinophilic phenotype in a human subject comprising orally administering to the not-well controlled asthmatic human subject a daily dose of about 75 mg to about 300 mg of dexpramipexole, or a pharmaceutically acceptable salt thereof, wherein the not-well controlled asthmatic human subject is already receiving at least two asthma medications, thereby treating the not-well controlled mild, moderate, and severe asthma of the eosinophilic phenotype in the not-well controlled asthmatic human subject.

In some embodiments, the not-well controlled moderate to severe asthmatic of the eosinophilic phenotype human subject is defined as a subject with Global Initiative for Asthma (GINA) steps 3-5 persistent asthma, requiring at minimum a low-dose inhaled corticosteroid in combination with a long-acting β-agonist, with a physician diagnosis of asthma for ≥12 months, and elevated AEC of ≥0.30×10⁹ cells/L. In some embodiments, the not-well controlled moderate to severe asthma of the eosinophilic phenotype is defined by the inclusion and exclusion criteria of Example 2 (paragraphs [0179], [0181], and [0182]). In some embodiments, the not-well controlled severe asthmatic of the eosinophilic phenotype human subject is defined by the inclusion and exclusion criteria of Example 4 (Table 15). In preferred embodiments, the not well-controlled mild asthmatic of the eosinophilic phenotype human subject exhibits one or more of the following criteria selected from the group consisting of requiring as needed (not daily) asthma treatment or no more than daily low-dose ICS treatment, ACQ score of ≥1.5, and combinations thereof. In preferred embodiments, the not well-controlled moderate asthmatic of the eosinophilic phenotype human subject exhibits one or more of the following criteria selected from the group consisting of requiring a daily low-dose ICS plus LABA or other controller treatment, ACQ score of ≥1.5, and combinations thereof. In preferred embodiments, the not well-controlled severe asthmatic of the eosinophilic phenotype human subject exhibits one or more of the following criteria selected from the group consisting of requiring a minimum daily medium-dose ICS plus LABA treatment, asthma exacerbations of 2 or more times a year, ACQ score of ≥1.5, and combinations thereof.

The inhaled corticosteroids (ICS) is selected from the group consisting of beclomethasone, fluticasone, ciclesonide, mometasone, budesonide, flunisolide, and combinations thereof. The inhaled corticosteroids may be medium-high dose inhaled corticosteroids or high-dose ICS. The long-acting beta agonist (LABA) is selected from the group consisting of albuterol sulfate, formoterol fumarate, salmeterol, salmeterol xinafoate, arformoterol tartrate, olodaterol, vilanterol, indacaterol, and combinations thereof. The amount of the inhaled corticosteroid being taken by the subject may be low dose, medium dose or high dose. Low-dose ICS refers to: about 100 μg to about 200 μg beclomethasone, about 320 μg flunisolide, about 88 μg to about 300 μg fluticasone is dependent on formulation, about 200 μg mometasone, about 200 μg to about 400 μg budesonide, and about 80 μg to about 160 μg ciclesonide. Medium-dose ICS refers to: about 300 μg to about 400 μg beclomethasone, about 320 μg to about 640 μg flunisolide, about 300 μg to about 500 μg fluticasone dependent on formulation, about 400 μg mometasone, about 600 μg to about 1200 μg budesonide, and about 240 μg to about 320 μg ciclesonide. High-dose ICS refers to: greater than about 40 μg beclomethasone, greater than about 640 μg flunisolide, greater than about 500 μg fluticasone dependent on formulation, greater than about 400 μg mometasone, greater than about 1200 μg budesonide, and about 400 μg to about 640 μg ciclesonide.

In embodiments described herein, the not-well controlled mild, moderate, to severe asthmatic of the eosinophilic phenotype human subject may further be taking an oral corticosteroid (OCS) selected from the group consisting of cortisone acetate, dexamethasone, hydrocortisone, methylprednisolone, prednisolone, prednisone, and combinations thereof. Maintenance oral corticosteroids (OCS) (up to 10 mg of prednisone per day or equivalent). In embodiments described herein, the not-well controlled mild, moderate to severe asthmatic of the eosinophilic phenotype subject may further be taking a long-acting muscarinic antagonist (LAMA) selected from the group consisting of umeclidinium, aclidinium, gycopyrronium, glycopyrrolate, tiotropium, and combinations thereof.

In some embodiments, the not-well controlled mild, moderate, and severe asthmatic of the eosinophilic phenotype human subject is 18 years of age or older. In some embodiments, the not-well controlled mild, moderate to severe asthmatic of the eosinophilic phenotype human subject is 12 years of age to about 17 years of age. In some embodiments, the not-well controlled mild, moderate to severe asthmatic of the eosinophilic phenotype human subject is about 6 years old to about 11 years old. In some embodiments, the not-well controlled mild, moderate to severe asthmatic of the eosinophilic phenotype human subject is about 2 years old to about 5 years old. In embodiments described herein, the not-well controlled mild, moderate to severe asthmatic of the eosinophilic phenotype human subject is an adult or an adolescent. In embodiments described herein, the adolescents is 12 up to 18 years of age. In embodiments described herein, the not-well controlled mild, moderate to severe asthmatic of the eosinophilic phenotype human subject is younger than 18 years of age. In embodiments described herein, the not-well controlled mild, moderate to severe asthmatic of the eosinophilic phenotype human subject is a child younger than 12 years of age. In embodiments described herein, the not-well controlled mild, moderate to severe asthmatic of the eosinophilic phenotype human subject is 18 years of age or older. In embodiments described herein, the not-well controlled mild, moderate to severe asthmatic of the eosinophilic phenotype human subject is between the ages of 18 to 75 years old. In embodiments described herein, the not-well controlled mild, moderate to severe asthmatic of the eosinophilic phenotype human subject is between the ages of 12 to 75 years old. In embodiments described herein, the not-well controlled mild, moderate to severe asthmatic of the eosinophilic phenotype human subject is 12 years of age or older.

In some embodiments, the not-well controlled mild, moderate to severe asthmatic of the eosinophilic phenotype human subject is treated, wherein the not-well controlled mild, moderate to severe asthmatic of the eosinophilic phenotype human subject shows improvement a measurement selected from the group consisting of forced expiratory volume in 1 second (FEV₁), score on Asthma Control Questionnaire (ACQ), score on Asthma Quality of Life Questionnaire (AQLQ), and combinations thereof.

In some embodiments, the not-well controlled mild, moderate to severe asthmatic of the eosinophilic phenotype human subject is treated, wherein the subject has a reduction in a symptom selected from the group consisting of level of nasal eosinophil peroxidase, level of pharyngeal eosinophil peroxidase, level of blood basophils, level of blood eosinophil progenitor population, fractional exhaled nitric oxide, and combinations thereof.

In some embodiments, the not-well controlled mild, moderate to severe asthmatic of the eosinophilic phenotype human subject is treated, wherein the subject shows improvement in reduction in frequency of asthma exacerbations.

In some embodiments, the not-well controlled mild, moderate to severe asthmatic of the eosinophilic phenotype human subject is treated, wherein the subject shows improvement in a measurement selected from the group consisting of forced expiratory volume in 1 second (FEV₁), forced vital capacity (FVC), annualized CompEx event rate, morning peak expiratory flow (PEF), score on Asthma Control Questionnaire (ACQ), score of Asthma Quality of Life Questionnaire (AQLQ), score on St. George's Respiratory Questionnaire, and combinations thereof.

In some embodiments, the not-well controlled mild, moderate to severe asthmatic of the eosinophilic phenotype human subject is treated, wherein the subject has a reduction in one or more of symptoms selected from the group consisting of time to first exacerbation, level of nasal eosinophil peroxidase, level of blood eosinophils, and combinations thereof.

In some embodiments, the not-well controlled mild, moderate to severe asthmatic of the eosinophilic phenotype human subject has a reduction in one or more of symptom selected from the group consisting of level of absolute blood eosinophil, level of tissue eosinophils, amount of mucus plugs in the airways, level of nasal eosinophil peroxidase, and combinations thereof.

In some embodiments, treating the not-well controlled mild, moderate to severe asthmatic of the eosinophilic phenotype human subject by oral administration of a daily dose of about 75 mg to about 300 mg of dexpramipexole results in a reduction of the level of eosinophils to less than 300 cells per microliter in the peripheral blood by a period of time selected from the group consisting of 12 weeks, 8 weeks, 6 weeks, 4 weeks, 3 weeks, or 2 weeks.

In some embodiments, treating the not-well controlled mild, moderate to severe asthmatic of the eosinophilic phenotype human subject by oral administration of a daily dose of about 75 mg to about 300 mg of dexpramipexole results in a reduction of the level of eosinophils to less than 250 cells per microliter in the peripheral blood by a period of time selected from the group consisting of 12 weeks, 8 weeks, 6 weeks, 4 weeks, 3 weeks, or 2 weeks.

In some embodiments, treating the not-well controlled mild, moderate to severe asthmatic of the eosinophilic phenotype human subject by oral administration of a daily dose of about 75 mg to about 300 mg of dexpramipexole results in a reduction of the level of eosinophils to less than 200 cells per microliter in the peripheral blood by a period of time selected from the group consisting of 12 weeks, 8 weeks, 6 weeks, 4 weeks, 3 weeks, or 2 weeks.

In some embodiments, treating the not-well controlled mild, moderate to severe asthmatic of the eosinophilic phenotype human subject by oral administration of a daily dose of about 75 mg to about 300 mg of dexpramipexole results in a reduction of the level of eosinophils to less than 150 cells per microliter in the peripheral blood by a period of time selected from the group consisting of 12 weeks, 8 weeks, 6 weeks, 4 weeks, 3 weeks, or 2 weeks.

In some embodiments, treating the not-well controlled mild, moderate to severe asthmatic of the eosinophilic phenotype human subject by oral administration of a daily dose of about 75 mg to about 300 mg of dexpramipexole results in a reduction of the level of eosinophils to less than 100 cells per microliter in the peripheral blood by a period of time selected from the group consisting of 12 weeks, 8 weeks, 6 weeks, 4 weeks, 3 weeks, or 2 weeks.

In some embodiments, treating the not-well controlled mild, moderate to severe asthmatic of the eosinophilic phenotype human subject by administration of a daily dose of about 75 mg to about 300 mg of dexpramipexole results in a reduction of the level of absolute blood eosinophils by at least about 90%. In certain embodiments, treating the not-well controlled mild, moderate to severe asthmatic of the eosinophilic phenotype human subject by administration of a daily dose of about 75 mg to about 300 mg of dexpramipexole results in a reduction of the level of absolute blood eosinophils by at least about 50%. In certain embodiments, treating the not-well controlled mild, moderate to severe asthmatic of the eosinophilic phenotype human subject by administration of a daily dose of about 300 mg of dexpramipexole results in a reduction of the level of absolute blood eosinophils by about 80%. In certain embodiments, treating the not-well controlled mild, moderate to severe asthmatic of the eosinophilic phenotype human subject by administration of a daily dose of about 150 mg of dexpramipexole results in a reduction of the level of absolute blood eosinophils by about 70%. In certain embodiments, treating the not-well controlled mild, moderate to severe asthmatic of the eosinophilic phenotype human subject by administration of a daily dose of about 75 mg of dexpramipexole results in a reduction of the level of absolute blood eosinophils by about 60%.

In some embodiments, treating the not-well controlled mild, moderate to severe asthmatic of the eosinophilic phenotype human subject by administration of a daily dose of about 150 mg to about 300 mg of dexpramipexole results in a reduction of the level of absolute blood eosinophils by at least about 90%. In certain embodiments, treating the not-well controlled mild, moderate to severe asthmatic of the eosinophilic phenotype human subject by administration of a daily dose of about 150 mg to about 300 mg of dexpramipexole results in a reduction of the level of absolute blood eosinophils by at least about 50%. In certain embodiments, treating the not-well controlled mild, moderate to severe asthmatic of the eosinophilic phenotype human subject by administration of a daily dose of about 300 mg of dexpramipexole results in a reduction of the level of absolute blood eosinophils by about 80%. In certain embodiments, treating the not-well controlled mild, moderate to severe asthmatic of the eosinophilic phenotype human subject by administration of a daily dose of about 150 mg of dexpramipexole results in a reduction of the level of absolute blood eosinophils by about 70%. In certain embodiments, treating the not-well controlled mild, moderate to severe asthmatic of the eosinophilic phenotype human subject by administration of a daily dose of about 75 mg of dexpramipexole results in a reduction of the level of absolute blood eosinophils by about 60%.

In some embodiments, treating the not-well controlled mild, moderate to severe asthmatic of the eosinophilic phenotype human subject results in a reduction of the level of eosinophils in the tissue. In certain embodiments, treating the not-well controlled mild, moderate to severe asthmatic of the eosinophilic phenotype human subject results in a reduction of the level of eosinophils in tissues including, but not limited to, lung tissue, nasal tissue, bone marrow, and combinations thereof. In certain embodiments, the level of eosinophils in the tissues are reduced by at least 40%, at least 45%, at least 50%, at least 55%, at least 60%, at least 65%, at least 70%, at least 75%, at least 80%, at least 85%, at least 90%, at least 95%, or 100%. In certain embodiments, the levels are reduced to normal. In certain embodiments, the levels are reduced to zero within the level of detection.

In some embodiments, treating the not-well controlled mild, moderate to severe asthmatic of the eosinophilic phenotype human subject results in a reduction in the amount of mucus plugs in the airways. Without wishing to be bound by theory, mucus plugs contribute to chronic airflow obstruction in severe asthma, and eosinophils release eosinophil peroxidase (EPX), which generates reactive oxygen species that oxidize cysteine thiol groups, this leads to cross-linking of the mucin proteins, making the mucus stiffer and promoting mucus plug formation in the bronchi (airways). In embodiments described herein, orally administering a daily dose of about 75 mg to about 300 mg of dexpramipexole, or a pharmaceutically acceptable salt thereof, decreases the level of tissue eosinophils, thus reducing airway eosinophil peroxidase, which in turn, reduces the amount of mucus plugs in the airways, all of which leads to improved lung function and greater exercise tolerance.

In some embodiments, the not-well controlled mild, moderate to severe asthmatic of the eosinophilic phenotype human subject is treated wherein the concentration of induced sputum eosinophil peroxidase (EPX), nasal eosinophil peroxidase (EPX), or pharyngeal eosinophil peroxidase (EPX) is reduced. Eosinophil peroxidase (EPX) is a granule protein uniquely secreted by eosinophils which can be detected and quantified using an enzyme-linked immunosorbent assay (ELISA). EPX is measured from mucosal swabs or sputum samples. In some embodiments, the moderate to severe asthma of the eosinophilic phenotype in a human subject is treated and the nasal EPX levels are reduced to normal levels. In embodiments, the nasal and sputum EPX levels following administration of a daily dose of about 75 mg to about 300 mg of dexpramipexole are reduced to level of the nasal and sputum EPX levels in a healthy, non-asthmatic control subject. In embodiments, normal nasal EPX levels are less than about 10 ng/mg of total protein, whereas moderate to severe asthma of the eosinophilic phenotype human subject may be diagnosed as having nasal EPX levels greater than about 10 ng/mg of total protein. In some embodiments, is treated and the induced sputum EPX levels are reduced to the levels observed in a healthy, non-asthmatic control subject. Normal induced sputum EPX levels may be less than about 200 ng/mL/g recovered sputum, whereas moderate to severe asthma of the eosinophilic phenotype human subject is diagnosed as having induced sputum EPX levels greater than about 200 ng/mL/g recovered sputum. In certain embodiments, treating the not-well controlled mild, moderate to severe asthmatic of the eosinophilic phenotype human subject by administration of a daily dose of about 300 mg of dexpramipexole results in a reduction of the level of nasal EPX by about 89%. In certain embodiments, treating the not-well controlled mild, moderate to severe asthmatic of the eosinophilic phenotype human subject by administration of a daily dose of about 150 mg of dexpramipexole results in a reduction of the level of nasal EPX by about 83%. In certain embodiments, treating the not-well controlled mild, moderate to severe asthmatic of the eosinophilic phenotype human subject by administration of a daily dose of about 75 mg of dexpramipexole results in a reduction of the level of nasal EPX by about 36%.

In some embodiments, is treated wherein the level of urine eosinophil granule proteins are reduced. In certain embodiments, the eosinophil granule proteins are selected from the group consisting of MBP-1 (Eosinophil Major Basic Protein-1), EDN (Eosinophil-derived Neurotoxin, RNase-2), ECP (Eosinophil Cationic Protein, RNase-3), EPX (Eosinophil Peroxidase), Charcot-Leyden Crystal protein (Gal-10/CLC) and combinations thereof.

In some embodiments, the reduction in blood eosinophils correlates with a reduction in tissue eosinophil biomarkers. In some embodiments, the tissue eosinophil biomarkers are selected from the group consisting of type 2 inflammation associated mediators, including IL-5, IL-13, IL-33, ST2 (IL1RL1), CCL2, CCL3, CCL4, CCL11, CCL17, CCR3, and combinations thereof. Eosinophil development is supported by a variety of cytokines, including Pc-related cytokines, such as GM-CSF, IL-3, and IL-5, which can also be used as biomarkers to assess the reduction in blood eosinophils. In some embodiments, the reduction in blood eosinophils correlates with a reduction in blood eosinophil progenitor populations. Eosinophils are derived from hematopoietic stem cells (HSCs), and both eosinophils and basophils are derived from myeloid progenitor cells.

In some embodiments, the methods of treating the not-well controlled mild, moderate to severe asthmatic of the eosinophilic phenotype human subject comprising orally administering a daily dose of about 75 mg to about 300 mg of dexpramipexole, wherein the level of blood basophils is reduced.

In some embodiments, the not-well controlled mild, moderate to severe asthmatic of the eosinophilic phenotype human subject is treated and the not-well controlled asthmatic human subject shows improvement in one or more of the measurements selected from the group consisting of reduction in the frequency of asthma exacerbations, reduction the use of oral corticosteroids, reduction in use of inhaled corticosteroids, reduction in use of long-acting beta agonist, improvement the mean forced expiratory volume in 1 second (FEV₁), improvement forced vital capacity (FVC), improvement in score on Asthma Control Questionnaire (ACQ), improvement in score on Asthma Control Test (ACT)™, improvement in score of Asthma Quality of Life Questionnaire (AQLQ), or a combination thereof.

In some embodiments, the not-well controlled mild, moderate to severe asthmatic of the eosinophilic phenotype human subject experienced at least 1 or at least 2 exacerbations over the past 12 months. A severe asthma exacerbation is defined as worsening of asthma requiring the use of systemic corticosteroids and/or hospitalization and/or medical intervention and/or emergency department (ED) visit. In certain embodiments, the medical intervention was corroborated with at least 1 of the following: 1) a decrease in forced expiratory volume in 1 second (FEV₁) by 20% or more from baseline, 2) a decrease in peak expiratory flow rate (PEFR) by 30% or more from baseline on 2 consecutive days, or 3) worsening of symptoms or other clinical signs per physician evaluation of the event. Systemic corticosteroid use is considered evidence of a severe asthma exacerbation for intravenous or oral routes if taken for at least 3 consecutive days; for the IM route, a single dose is sufficient. In embodiments, an exacerbation is defined as the worsening of asthma symptoms and lung function requiring use of oral/systemic corticosteroids for at least 3 days. In embodiments, for subjects on maintenance oral corticosteroids, an exacerbation requiring oral/systemic corticosteroids is defined as the use of oral/systemic corticosteroids that is at least double the existing dose for at least 3 days. In embodiments, frequency of exacerbations is defined as the number of exacerbations per a particular defined unit of time, such as 8 weeks, 12 weeks, 24 weeks, 26 weeks, 36 weeks, or 52 weeks. In embodiments described herein, exacerbations requiring the use of a systemic corticosteroid (e.g., OCS) as well as exacerbations resulting in hospitalization or an emergency room visit were each reduced. In some embodiments, treating the not-well controlled mild, moderate to severe asthmatic of the eosinophilic phenotype human subject by administration of a daily dose of about 75 mg to about 300 mg of dexpramipexole results in a reduction in the number of exacerbations per year. In some embodiments, improvement in exacerbations measured by either fewer exacerbations per unit of time or an extension in the time between the next subsequent exacerbation. In some embodiments, the exacerbation rate is reduced to less than 2 per year, less than 1 per year, or is 0 per year. In some embodiments, the time to first severe asthma exacerbation was extended. In some embodiments, the time to first severe asthma exacerbation did not occur within one year of the previous exacerbation.

In some embodiments, treating the not-well controlled mild, moderate to severe asthmatic of the eosinophilic phenotype human subject by administration of a daily dose of about 75 mg to about 300 mg of dexpramipexole results in improvement in the subject's annualized CompEx event rate. Annualized CompEx event rate is a measurement of an extended definition of asthma exacerbations, combining diary-based events with traditionally defined severe exacerbations. Diary events are based on objective measures of deterioration of peak expiratory flow, reliever use, and asthma symptoms assessed morning and evening and night-time awakenings. Deterioration is defined as either reaching a predefined change from baseline (threshold), for at least 2 consecutive days, or deterioration in all variables over at least a 5-day period plus at least one variable reaching a threshold criterion for at least 2 consecutive days. Deterioration of at least two concurrent criteria is needed to fulfill the criteria for a diary event.

In some embodiments, treating the not-well controlled mild, moderate to severe asthmatic of the eosinophilic phenotype human subject by administration of a daily dose of about 75 mg to about 300 mg of dexpramipexole results in a reduction in oral corticosteroid dose. In some embodiments, the oral corticosteroid dose is reduced by about 90% to about 100%, about 75% to about 90%, about 50% to about 75%, or about 10% to about 50%. In some embodiments, the oral corticosteroid dose is reduced to zero. In some embodiments, the frequency of the use of oral corticosteroid is reduced from baseline, such as a 90-100% decrease, a 75-90% decrease, a 50-75% decrease, or at least a 50% reduction. In embodiments described herein, use of oral corticosteroids is reduced to every other day. In embodiments described herein, reductions in oral corticosteroid dose is gradual and performed under the supervision of a physician. In embodiments described herein, reductions in oral corticosteroid dose and frequency occurs while maintaining asthma control.

In some embodiments, treating the not-well controlled mild, moderate to severe asthmatic of the eosinophilic phenotype human subject by administration of a daily dose of about 75 mg to about 300 mg of dexpramipexole results in a reduction in inhaled corticosteroid dose. In some embodiments, the inhaled corticosteroid dose is reduced by about 90% to about 100%, about 75% to about 90%, about 50% to about 75%, or about 10% to about 50%. In some embodiments, the inhaled corticosteroid dose is reduced to zero. In some embodiments, the frequency of the use of inhaled corticosteroid is reduced from baseline, such as a 90-100% decrease, a 75-90% decrease, a 50-75% decrease, or at least a 50% reduction. In embodiments described herein, the use of inhaled corticosteroids is reduced to less than 3 times per week. In embodiments described herein, reductions in inhaled corticosteroid dose is gradual and performed under the supervision of a physician. In embodiments described herein, reductions in inhaled corticosteroid dose and frequency occurs while maintaining asthma control.

In some embodiments, treating the not-well controlled mild, moderate to severe asthmatic of the eosinophilic phenotype human subject by administration of a daily dose of about 75 mg to about 300 mg of dexpramipexole results in a reduction in long-acting beta agonist dose. In some embodiments, the long-acting beta agonist dose is reduced by about 90% to about 100%, about 75% to about 90%, about 50% to about 75%, or about 10% to about 50%. In some embodiments, the long-acting beta agonist dose is reduced to zero. In some embodiments, the frequency of the use of long-acting beta agonist is reduced from baseline, such as a 90-100% decrease, a 75-90% decrease, a 50-75% decrease, or at least a 50% reduction. In embodiments described herein, the use of long-acting beta agonist is reduced to less than 1 time per day. In embodiments described herein, reductions in long-acting beta agonist dose is gradual and performed under the supervision of a physician. In embodiments described herein, reductions in long-acting beta agonist dose and frequency occurs while maintaining asthma control.

Embodiments described herein are directed to methods of treating the not-well controlled mild, moderate to severe asthmatic of the eosinophilic phenotype human subject comprising orally administering a daily dose of about 75 mg to about 300 mg of dexpramipexole, wherein pulmonary function is improved. Pulmonary function is measured by performing a spirometry, which measures forced expiratory volume in is (FEV₁), the volume of air moved in the first second of exhalation, this is a well-accepted measure of asthma. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

Embodiments described herein are directed to methods of treating the not-well controlled mild, moderate to severe asthmatic of the eosinophilic phenotype human subject comprising orally administering a daily dose of about 75 mg to about 300 mg of dexpramipexole, wherein FEV₁ is improved. In certain embodiments, the not-well controlled asthmatic human subject had a baseline FEV₁ of about 1.9 L to about 2.2 L at baseline, which is about a 60% reduction from normal/predicted. In some embodiments, the FEV₁ is increased by about 5% to about 20%. More preferably, the FEV₁ is increased by about 10% to about 20%. In some embodiments, the volume of FEV₁ is increased by about 150 ml to about 300 ml. More preferably, the FEV₁ is increased by about 200 ml to about 300 ml.

In some embodiments, treating the not-well controlled mild, moderate to severe asthmatic of the eosinophilic phenotype human subject by administration of a daily dose of about 75 mg to about 300 mg of dexpramipexole results in an increase in the volume of FEV₁ in about 12 weeks, about 8 weeks, about 6 weeks, about 4 weeks, about 3 weeks, or about 2 weeks.

Embodiments described herein are directed to methods of treating the not-well controlled mild, moderate to severe asthmatic of the eosinophilic phenotype human subject comprising orally administering a daily dose of about 75 mg to about 300 mg of dexpramipexole, wherein FVC is improved. In certain embodiments, the not-well controlled asthmatic human subject had a baseline FVC of about 3.0 L to about 3.3 L, which is about a 74%-80% reduction from normal/predicted. In some embodiments, the FVC is increased by about 2% to about 12%. More preferably, the FVC is increased by about 8% to about 12%. In some embodiments, the volume of FVC is increased by about 20 ml to about 450 ml. More preferably, the volume of FVC is increased by about 200 ml to about 450 ml.

In some embodiments, treating the not-well controlled mild, moderate to severe asthmatic of the eosinophilic phenotype human subject by administration of a daily dose of about 75 mg to about 300 mg of dexpramipexole results in an increase in the volume of FVC in about 12 weeks, about 8 weeks, about 6 weeks, about 4 weeks, about 3 weeks, or about 2 weeks.

In some embodiments, treating the not-well controlled mild, moderate to severe asthmatic of the eosinophilic phenotype human subject by administration of a daily dose of about 75 mg to about 300 mg of dexpramipexole results in an increase in morning peak expiratory flow (PEF). Peak flow zones are areas of measurement on a peak flow meter. The goal of the peak flow zones is to show early symptoms of uncontrolled asthma. The green zone is 80% to 100% of the subject's highest peak flow reading. The yellow zone is 50% to 80% of the subject's highest peak flow reading. Measurements in this zone are a sign that large airways are starting to narrow and subject may start to have mild symptoms, such as coughing, feeling tired, feeling short of breath, or chest tightening. The red zone is less than 50% of the subject's highest peak flow reading. Readings in this zone mean severe narrowing of large airways, which is a medical emergency. In some embodiments, treating the not-well controlled mild, moderate to severe asthmatic of the eosinophilic phenotype human subject by administration of a daily dose of about 75 mg to about 300 mg of dexpramipexole results in the subject's ability to remain in the green zone when measuring morning peak expiratory flow.

Airway inflammation can also be assessed using the FeNO test, fractional concentration of exhaled nitric oxide, wherein the level of nitric oxide is measured in parts per billion (ppb) in the air you slowly exhale. Higher than normal levels of exhaled nitric oxide generally means the airways are inflamed, a sign of asthma. Levels less than about 20 ppb in children and less than about 25 ppb in adults are considered normal. Greater than about 35 ppb in children and 50 ppb in adults are considered to be abnormal.

Embodiments described herein are directed to methods of treating the not-well controlled mild, moderate to severe asthmatic of the eosinophilic phenotype human subject comprising orally administering a daily dose of about 75 mg to about 300 mg of dexpramipexole, wherein the not-well controlled mild, moderate to severe asthmatic of the eosinophilic phenotype human subject is controlled. The Asthma Control Questionnaire (ACQ), and/or the Asthma Control Test (ACT)™ can be used to assess whether asthma is being controlled. In certain embodiments, the not-well controlled asthmatic human subject had a baseline score of about 1.9 to about 2.3 on the ACQ and about 4.5 to about 5.4 on the AQLQ.

The ACQ cutoff for controlled asthma is a score of about 0.75, and the ACQ cutoff for uncontrolled asthma is a score of about 1.5. An ACQ score change of ≥0.5 is considered clinically significant. In embodiments described herein, the not-well controlled asthmatic human subject has a change in ACQ score of at least 0.5 points. In embodiments described herein, the not-well controlled asthmatic human subject has an improvement in score on ACQ and the score is less than or equal to 0.75.

Using the Asthma Control Test (ACT)™, a score of less than 19 indicates the individual's asthma may not be well controlled and a score of less than 15 indicates the individual's asthma may be very poorly controlled. In embodiments described herein, the not-well controlled asthmatic human subject has a change in ACT score of at least 0.5 points. In embodiments described herein, the not-well controlled asthmatic human subject has an improvement in score on Asthma Control Test (ACT)™ and the score is greater than 19.

The AQLQ is a 32-item asthma specific questionnaire designed to measure functional impairments that are most important to patients with asthma. The 32 questions in the AQLQ are divided into four domains; activity limitations, symptoms, emotional function, and environmental stimuli. Individual questions are equally weighted. The overall AQLQ score is the mean of the responses to each of the 32 questions and ranges from 1 to 7. A score 7.0 indicates that the patient has no impairments due to asthma and score 1.0 indicates severe impairment. Embodiments described herein are directed to methods of treating the not-well controlled mild, moderate to severe asthmatic of the eosinophilic phenotype human subject comprising orally administering a daily dose of about 75 mg to about 300 mg of dexpramipexole, wherein the subject's quality of life is improved. The Asthma Quality of Life Questionnaire (AQLQ) can be used to assess an individual's quality of life, scores range from 1-7, with higher scores indicating better quality of life. In embodiments described herein, the not-well controlled asthmatic human subject has a change in AQLQ score of at least 0.5 points. In embodiments described herein, the not-well controlled asthmatic human subject has an improvement in score on Asthma Quality of Life Questionnaire (AQLQ) and the score is greater than 3.5.

In embodiments described herein, the not-well controlled asthmatic human subject has an improvement in score on the St. George's Respiratory Questionnaire.

In some embodiments, treating the not-well controlled mild, moderate to severe asthmatic of the eosinophilic phenotype human subject by administration of a daily dose of about 75 mg to about 300 mg of dexpramipexole results in improving a subject's overall morbidity. In some embodiments, treating the not-well controlled mild, moderate to severe asthmatic of the eosinophilic phenotype human subject by administration of a daily dose of about 75 mg to about 300 mg of dexpramipexole results in improving a subject's overall mortality risk.

Uncontrolled Severe Asthma

Embodiments are directed to methods for treating uncontrolled severe asthma of the eosinophilic phenotype in a human subject comprising orally administering to the uncontrolled asthmatic human subject a daily dose of about 75 mg to about 300 mg of dexpramipexole, or a pharmaceutically acceptable salt thereof, wherein the uncontrolled asthmatic human subject is already receiving at least two asthma medications, thereby treating the uncontrolled severe asthma of the eosinophilic phenotype in the uncontrolled asthmatic human subject.

In some embodiments, the uncontrolled severe human subject is defined as a subject with symptomatic severe asthmatic as defined by GINA steps 4 and 5 requiring daily treatment with, at a minimum, medium dose inhaled corticosteroids in combination with a long-acting β2 agonist, for at least 3 months and on a stable dose for at least 1 month, having an FEV₁ of <80% of predicted, as evidenced by ≥12% and ≥200 mL improvement in FEV₁ 15 to 25 minutes following inhalation of albuterol, pre-bronchodilator FEV_{1 ≥40}%, ACQ-6 ≥1.5, and documented history of ≥2 asthma exacerbations requiring treatment with systemic corticosteroids (intramuscular, intravenous, or oral), in the previous 12 months. In some embodiments, the uncontrolled severe asthma of the eosinophilic phenotype is defined by the inclusion and exclusion criteria of Example 4 (Table 15). In preferred embodiments, the important criteria include one or more selected from the group consisting of requiring a minimum daily medium dose ICS plus LABA treatment to maintain asthma control, severe asthma exacerbations of 2 or more times a year, ACQ score of ≥1.5, and AEC of ≥0.30×10⁹ cells/L.

The inhaled corticosteroids (ICS) is selected from the group consisting of beclomethasone, fluticasone, ciclesonide, mometasone, budesonide, flunisolide, and combinations thereof. The inhaled corticosteroids may be medium-high dose inhaled corticosteroids or high-dose ICS. The long-acting beta agonist (LABA) is selected from the group consisting of albuterol sulfate, formoterol fumarate, salmeterol, salmeterol xinafoate, arformoterol tartrate, olodaterol, vilanterol, indacaterol, and combinations thereof. The amount of the inhaled corticosteroid being taken by the subject may be low dose, medium dose or high dose.

In embodiments described herein, the uncontrolled severe asthma of the eosinophilic phenotype in the subject may further be taking an oral corticosteroid (OCS) selected from the group consisting of cortisone acetate, dexamethasone, hydrocortisone, methylprednisolone, prednisolone, prednisone, and combinations thereof. Maintenance oral corticosteroids (OCS) (up to 10 mg of prednisone per day or equivalent). In embodiments described herein, the uncontrolled severe asthma of the eosinophilic phenotype in the subject may further be taking a long-acting muscarinic antagonist (LAMA) selected from the group consisting of umeclidinium, aclidinium, gycopyrronium, glycopyrrolate, tiotropium, and combinations thereof.

In some embodiments, the uncontrolled severe asthma of the eosinophilic phenotype in the subject is 18 years of age or older. In some embodiments, the uncontrolled severe asthma of the eosinophilic phenotype in the subject is 12 years of age to about 17 years of age. In some embodiments, the uncontrolled severe asthma of the eosinophilic phenotype in the subject is about 6 years old to about 11 years old. In some embodiments, the uncontrolled severe asthma of the eosinophilic phenotype in the subject is about 2 years old to about 5 years old. In embodiments described herein, the uncontrolled severe asthma of the eosinophilic phenotype in the subject is an adult or an adolescent. In embodiments described herein, the adolescents is 12 up to 18 years of age. In embodiments described herein, the uncontrolled severe asthma of the eosinophilic phenotype in the subject is younger than 18 years of age. In embodiments described herein, the uncontrolled severe asthma of the eosinophilic phenotype in the subject is a child younger than 12 years of age. In embodiments described herein, the uncontrolled severe asthma of the eosinophilic phenotype in the subject is 18 years of age or older. In embodiments described herein, the uncontrolled severe asthma of the eosinophilic phenotype in the subject is between the ages of 18 to 75 years old. In embodiments described herein, the uncontrolled severe asthma of the eosinophilic phenotype in the subject is between the ages of 12 to 75 years old. In embodiments described herein, the uncontrolled severe asthma of the eosinophilic phenotype in the subject is 12 years of age or older.

In some embodiments, the uncontrolled severe asthma of the eosinophilic phenotype in the subject is treated, wherein the uncontrolled severe asthma of the eosinophilic phenotype in the subject shows improvement a measurement selected from the group consisting of forced expiratory volume in 1 second (FEV₁), score on Asthma Control Questionnaire (ACQ), score on Asthma Quality of Life Questionnaire (AQLQ), and combinations thereof.

In some embodiments, the uncontrolled severe asthma of the eosinophilic phenotype in the subject is treated, wherein the subject has a reduction in a symptom selected from the group consisting of level of nasal eosinophil peroxidase, level of pharyngeal eosinophil peroxidase, level of blood basophils, level of blood eosinophil progenitor population, fractional exhaled nitric oxide, and combinations thereof.

In some embodiments, the uncontrolled severe asthma of the eosinophilic phenotype in the subject is treated, wherein the subject shows improvement in reduction in frequency of asthma exacerbations.

In some embodiments, the uncontrolled severe asthma of the eosinophilic phenotype in the subject is treated, wherein the subject shows improvement in a measurement selected from the group consisting of forced expiratory volume in 1 second (FEV1), forced vital capacity (FVC), annualized CompEx event rate, morning peak expiratory flow (PEF), score on Asthma Control Questionnaire (ACQ), score of Asthma Quality of Life Questionnaire (AQLQ), score on St. George's Respiratory Questionnaire, and combinations thereof.

In some embodiments, the uncontrolled severe asthma of the eosinophilic phenotype in the subject is treated, wherein the subject has a reduction in one or more of symptoms selected from the group consisting of time to first exacerbation, level of nasal eosinophil peroxidase, level of blood eosinophils, and combinations thereof.

In some embodiments, the uncontrolled severe asthma of the eosinophilic phenotype in the subject has a reduction in one or more of symptom selected from the group consisting of level of absolute blood eosinophil, level of tissue eosinophils, amount of mucus plugs in the airways, level of nasal eosinophil peroxidase, and combinations thereof.

In some embodiments, treating the uncontrolled severe asthma of the eosinophilic phenotype in the subject by oral administration of a daily dose of about 75 mg to about 300 mg of dexpramipexole results in a reduction of the level of eosinophils to less than 300 cells per microliter in the peripheral blood by a period of time selected from the group consisting of 12 weeks, 8 weeks, 6 weeks, 4 weeks, 3 weeks, or 2 weeks.

In some embodiments, treating the uncontrolled severe asthma of the eosinophilic phenotype in the subject by oral administration of a daily dose of about 75 mg to about 300 mg of dexpramipexole results in a reduction of the level of eosinophils to less than 250 cells per microliter in the peripheral blood by a period of time selected from the group consisting of 12 weeks, 8 weeks, 6 weeks, 4 weeks, 3 weeks, or 2 weeks.

In some embodiments, treating the uncontrolled severe asthma of the eosinophilic phenotype in the subject by oral administration of a daily dose of about 75 mg to about 300 mg of dexpramipexole results in a reduction of the level of eosinophils to less than 200 cells per microliter in the peripheral blood by a period of time selected from the group consisting of 12 weeks, 8 weeks, 6 weeks, 4 weeks, 3 weeks, or 2 weeks.

In some embodiments, treating the uncontrolled severe asthma of the eosinophilic phenotype in the subject by oral administration of a daily dose of about 75 mg to about 300 mg of dexpramipexole results in a reduction of the level of eosinophils to less than 150 cells per microliter in the peripheral blood by a period of time selected from the group consisting of 12 weeks, 8 weeks, 6 weeks, 4 weeks, 3 weeks, or 2 weeks.

In some embodiments, treating the uncontrolled severe asthma of the eosinophilic phenotype in the subject by oral administration of a daily dose of about 75 mg to about 300 mg of dexpramipexole results in a reduction of the level of eosinophils to less than 100 cells per microliter in the peripheral blood by a period of time selected from the group consisting of 12 weeks, 8 weeks, 6 weeks, 4 weeks, 3 weeks, or 2 weeks.

In some embodiments, treating the uncontrolled severe asthma of the eosinophilic phenotype in the subject by administration of a daily dose of about 75 mg to about 300 mg of dexpramipexole results in a reduction of the level of absolute blood eosinophils by at least about 90%. In certain embodiments, treating the uncontrolled severe asthma of the eosinophilic phenotype in the subject by administration of a daily dose of about 75 mg to about 300 mg of dexpramipexole results in a reduction of the level of absolute blood eosinophils by at least about 50%. In certain embodiments, treating the uncontrolled severe asthma of the eosinophilic phenotype in the subject by administration of a daily dose of about 300 mg of dexpramipexole results in a reduction of the level of absolute blood eosinophils by about 80%. In certain embodiments, treating the uncontrolled severe asthma of the eosinophilic phenotype in the subject by administration of a daily dose of about 150 mg of dexpramipexole results in a reduction of the level of absolute blood eosinophils by about 70%. In certain embodiments, treating the uncontrolled severe asthma of the eosinophilic phenotype in the subject by administration of a daily dose of about 75 mg of dexpramipexole results in a reduction of the level of absolute blood eosinophils by about 60%.

In some embodiments, treating the uncontrolled severe asthma of the eosinophilic phenotype in the subject by administration of a daily dose of about 150 mg to about 300 mg of dexpramipexole results in a reduction of the level of absolute blood eosinophils by at least about 90%. In certain embodiments, treating the uncontrolled severe asthma of the eosinophilic phenotype in the subject by administration of a daily dose of about 150 mg to about 300 mg of dexpramipexole results in a reduction of the level of absolute blood eosinophils by at least about 50%. In certain embodiments, treating the uncontrolled severe asthma of the eosinophilic phenotype in the subject by administration of a daily dose of about 300 mg of dexpramipexole results in a reduction of the level of absolute blood eosinophils by about 80%. In certain embodiments, treating the uncontrolled severe asthma of the eosinophilic phenotype in the subject by administration of a daily dose of about 150 mg of dexpramipexole results in a reduction of the level of absolute blood eosinophils by about 70%. In certain embodiments, treating the uncontrolled severe asthma of the eosinophilic phenotype in the subject by administration of a daily dose of about 75 mg of dexpramipexole results in a reduction of the level of absolute blood eosinophils by about 60%.

In some embodiments, treating the uncontrolled severe asthma of the eosinophilic phenotype in the subject results in a reduction of the level of eosinophils in the tissue. In certain embodiments, treating the uncontrolled severe asthma of the eosinophilic phenotype in the subject results in a reduction of the level of eosinophils in tissues including, but not limited to, lung tissue, nasal tissue, bone marrow, and combinations thereof. In certain embodiments, the level of eosinophils in the tissues are reduced by at least 40%, at least 45%, at least 50%, at least 55%, at least 60%, at least 65%, at least 70%, at least 75%, at least 80%, at least 85%, at least 90%, at least 95%, or 100%. In certain embodiments, the levels are reduced to normal. In certain embodiments, the levels are reduced to zero within the level of detection.

In some embodiments, treating the uncontrolled severe asthma of the eosinophilic phenotype in the subject results in a reduction in the amount of mucus plugs in the airways. Without wishing to be bound by theory, mucus plugs contribute to chronic airflow obstruction in severe asthma, and eosinophils release eosinophil peroxidase (EPX), which generates reactive oxygen species that oxidize cysteine thiol groups, this leads to cross-linking of the mucin proteins, making the mucus stiffer and promoting mucus plug formation in the bronchi (airways). In embodiments described herein, orally administering a daily dose of about 75 mg to about 300 mg of dexpramipexole, or a pharmaceutically acceptable salt thereof, decreases the level of tissue eosinophils, thus reducing airway eosinophil peroxidase, which in turn, reduces the amount of mucus plugs in the airways, all of which leads to improved lung function and greater exercise tolerance.

In some embodiments, the uncontrolled severe asthma of the eosinophilic phenotype in the subject is treated wherein the concentration of induced sputum eosinophil peroxidase (EPX), nasal eosinophil peroxidase (EPX), or pharyngeal eosinophil peroxidase (EPX) is reduced. Eosinophil peroxidase (EPX) is a granule protein uniquely secreted by eosinophils which can be detected and quantified using an enzyme-linked immunosorbent assay (ELISA). EPX is measured from mucosal swabs or sputum samples. In some embodiments, the moderate to severe asthma of the eosinophilic phenotype in a human subject is treated and the nasal EPX levels are reduced to normal levels. In embodiments, the nasal and sputum EPX levels following administration of a daily dose of about 75 mg to about 300 mg of dexpramipexole are reduced to level of the nasal and sputum EPX levels in a healthy, non-asthmatic control subject. In embodiments, normal nasal EPX levels are less than about 10 ng/mg of total protein, whereas moderate to severe asthma of the eosinophilic phenotype human subject may be diagnosed as having nasal EPX levels greater than about 10 ng/mg of total protein. In some embodiments, is treated and the induced sputum EPX levels are reduced to the levels observed in a healthy, non-asthmatic control subject. Normal induced sputum EPX levels may be less than about 200 ng/mL/g recovered sputum, whereas moderate to severe asthma of the eosinophilic phenotype human subject is diagnosed as having induced sputum EPX levels greater than about 200 ng/mL/g recovered sputum. In certain embodiments, treating the uncontrolled severe asthma of the eosinophilic phenotype in the subject by administration of a daily dose of about 300 mg of dexpramipexole results in a reduction of the level of nasal EPX by about 89%. In certain embodiments, treating the uncontrolled severe asthma of the eosinophilic phenotype in the subject by administration of a daily dose of about 150 mg of dexpramipexole results in a reduction of the level of nasal EPX by about 83%. In certain embodiments, treating the uncontrolled severe asthma of the eosinophilic phenotype in the subject by administration of a daily dose of about 75 mg of dexpramipexole results in a reduction of the level of nasal EPX by about 36%.

In some embodiments, is treated wherein the level of urine eosinophil granule proteins are reduced. In certain embodiments, the eosinophil granule proteins are selected from the group consisting of MBP-1 (Eosinophil Major Basic Protein-1), EDN (Eosinophil-derived Neurotoxin, RNase-2), ECP (Eosinophil Cationic Protein, RNase-3), EPX (Eosinophil Peroxidase), Charcot-Leyden Crystal protein (Gal-10/CLC) and combinations thereof.

In some embodiments, the reduction in blood eosinophils correlates with a reduction in tissue eosinophil biomarkers. In some embodiments, the tissue eosinophil biomarkers are selected from the group consisting of type 2 inflammation associated mediators, including IL-5, IL-13, IL-33, ST2 (IL1RL1), CCL2, CCL3, CCL4, CCL11, CCL17, CCR3, and combinations thereof. Eosinophil development is supported by a variety of cytokines, including Pc-related cytokines, such as GM-CSF, IL-3, and IL-5, which can also be used as biomarkers to assess the reduction in blood eosinophils. In some embodiments, the reduction in blood eosinophils correlates with a reduction in blood eosinophil progenitor populations. Eosinophils are derived from hematopoietic stem cells (HSCs), and both eosinophils and basophils are derived from myeloid progenitor cells.

In some embodiments, the methods of treating the uncontrolled severe asthma of the eosinophilic phenotype in the subject comprising orally administering a daily dose of about 75 mg to about 300 mg of dexpramipexole, wherein the level of blood basophils is reduced.

In some embodiments, the uncontrolled severe asthma of the eosinophilic phenotype in the subject is treated and the uncontrolled asthmatic human subject shows improvement in one or more of the measurements selected from the group consisting of reduction in the frequency of asthma exacerbations, reduction the use of oral corticosteroids, reduction in use of inhaled corticosteroids, reduction in use of long-acting beta agonist, improvement the mean forced expiratory volume in 1 second (FEV₁), improvement forced vital capacity (FVC), improvement in score on Asthma Control Questionnaire (ACQ), improvement in score on Asthma Control Test (ACT)™, improvement in score of Asthma Quality of Life Questionnaire (AQLQ), or a combination thereof.

In some embodiments, the uncontrolled severe asthma of the eosinophilic phenotype in the subject experienced at least 1 or at least 2 exacerbations over the past 12 months. A severe asthma exacerbation is defined as worsening of asthma requiring the use of systemic corticosteroids and/or hospitalization and/or medical intervention and/or emergency department (ED) visit. In certain embodiments, the medical intervention was corroborated with at least 1 of the following: 1) a decrease in forced expiratory volume in 1 second (FEV₁) by 20% or more from baseline, 2) a decrease in peak expiratory flow rate (PEFR) by 30% or more from baseline on 2 consecutive days, or 3) worsening of symptoms or other clinical signs per physician evaluation of the event. Systemic corticosteroid use is considered evidence of a severe asthma exacerbation for intravenous or oral routes if taken for at least 3 consecutive days; for the IM route, a single dose is sufficient. In embodiments, an exacerbation is defined as the worsening of asthma symptoms and lung function requiring use of oral/systemic corticosteroids for at least 3 days. In embodiments, for subjects on maintenance oral corticosteroids, an exacerbation requiring oral/systemic corticosteroids is defined as the use of oral/systemic corticosteroids that is at least double the existing dose for at least 3 days. In embodiments, frequency of exacerbations is defined as the number of exacerbations per a particular defined unit of time, such as 8 weeks, 12 weeks, 24 weeks, 26 weeks, 36 weeks, or 52 weeks. In embodiments described herein, exacerbations requiring the use of a systemic corticosteroid (e.g., OCS) as well as exacerbations resulting in hospitalization or an emergency room visit were each reduced. In some embodiments, treating the uncontrolled severe asthma of the eosinophilic phenotype in the subject by administration of a daily dose of about 75 mg to about 300 mg of dexpramipexole results in a reduction in the number of exacerbations per year. In some embodiments, improvement in exacerbations measured by either fewer exacerbations per unit of time or an extension in the time between the next subsequent exacerbation. In some embodiments, the exacerbation rate is reduced to less than 2 per year, less than 1 per year, or is 0 per year. In some embodiments, the time to first severe asthma exacerbation was extended. In some embodiments, the time to first severe asthma exacerbation did not occur within one year of the previous exacerbation.

In some embodiments, treating the uncontrolled severe asthma of the eosinophilic phenotype in the subject by administration of a daily dose of about 75 mg to about 300 mg of dexpramipexole results in improvement in the subject's annualized CompEx event rate. Annualized CompEx event rate is a measurement of an extended definition of asthma exacerbations, combining diary-based events with traditionally defined severe exacerbations. Diary events are based on objective measures of deterioration of peak expiratory flow, reliever use, and asthma symptoms assessed morning and evening and night-time awakenings. Deterioration is defined as either reaching a predefined change from baseline (threshold), for at least 2 consecutive days, or deterioration in all variables over at least a 5-day period plus at least one variable reaching a threshold criterion for at least 2 consecutive days. Deterioration of at least two concurrent criteria is needed to fulfill the criteria for a diary event.

In some embodiments, treating the uncontrolled severe asthma of the eosinophilic phenotype in the subject by administration of a daily dose of about 75 mg to about 300 mg of dexpramipexole results in a reduction in oral corticosteroid dose. In some embodiments, the oral corticosteroid dose is reduced by about 90% to about 100%, about 75% to about 90%, about 50% to about 75%, or about 10% to about 50%. In some embodiments, the oral corticosteroid dose is reduced to zero. In some embodiments, the frequency of the use of oral corticosteroid is reduced from baseline, such as a 90-100% decrease, a 75-90% decrease, a 50-75% decrease, or at least a 50% reduction. In embodiments described herein, use of oral corticosteroids is reduced to every other day. In embodiments described herein, reductions in oral corticosteroid dose is gradual and performed under the supervision of a physician. In embodiments described herein, reductions in oral corticosteroid dose and frequency occurs while maintaining asthma control.

In some embodiments, treating the uncontrolled severe asthma of the eosinophilic phenotype in the subject by administration of a daily dose of about 75 mg to about 300 mg of dexpramipexole results in a reduction in inhaled corticosteroid dose. In some embodiments, the inhaled corticosteroid dose is reduced by about 90% to about 100%, about 75% to about 90%, about 50% to about 75%, or about 10% to about 50%. In some embodiments, the inhaled corticosteroid dose is reduced to zero. In some embodiments, the frequency of the use of inhaled corticosteroid is reduced from baseline, such as a 90-100% decrease, a 75-90% decrease, a 50-75% decrease, or at least a 50% reduction. In embodiments described herein, the use of inhaled corticosteroids is reduced to less than 3 times per week. In embodiments described herein, reductions in inhaled corticosteroid dose is gradual and performed under the supervision of a physician. In embodiments described herein, reductions in inhaled corticosteroid dose and frequency occurs while maintaining asthma control.

In some embodiments, treating the uncontrolled severe asthma of the eosinophilic phenotype in the subject by administration of a daily dose of about 75 mg to about 300 mg of dexpramipexole results in a reduction in long-acting beta agonist dose. In some embodiments, the long-acting beta agonist dose is reduced by about 90% to about 100%, about 75% to about 90%, about 50% to about 75%, or about 10% to about 50%. In some embodiments, the long-acting beta agonist dose is reduced to zero. In some embodiments, the frequency of the use of long-acting beta agonist is reduced from baseline, such as a 90-100% decrease, a 75-90% decrease, a 50-75% decrease, or at least a 50% reduction. In embodiments described herein, the use of long-acting beta agonist is reduced to less than 1 time per day. In embodiments described herein, reductions in long-acting beta agonist dose is gradual and performed under the supervision of a physician. In embodiments described herein, reductions in long-acting beta agonist dose and frequency occurs while maintaining asthma control.

Embodiments described herein are directed to methods of treating the uncontrolled severe asthma of the eosinophilic phenotype in the subject comprising orally administering a daily dose of about 75 mg to about 300 mg of dexpramipexole, wherein pulmonary function is improved. Pulmonary function is measured by performing a spirometry, which measures forced expiratory volume in 1s (FEV₁), the volume of air moved in the first second of exhalation, this is a well-accepted measure of asthma. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

Embodiments described herein are directed to methods of treating the uncontrolled severe asthma of the eosinophilic phenotype in the subject comprising orally administering a daily dose of about 75 mg to about 300 mg of dexpramipexole, wherein FEV₁ is improved. In certain embodiments, the uncontrolled asthmatic human subject had a baseline FEV₁ of about 1.9 L to about 2.2 L at baseline, which is about a 60% reduction from normal/predicted. In some embodiments, the FEV₁ is increased by about 5% to about 20%. More preferably, the FEV₁ is increased by about 10% to about 20%. In some embodiments, the volume of FEV₁ is increased by about 150 ml to about 300 ml. More preferably, the FEV₁ is increased by about 200 ml to about 300 ml.

In some embodiments, treating the uncontrolled severe asthma of the eosinophilic phenotype in the subject by administration of a daily dose of about 75 mg to about 300 mg of dexpramipexole results in an increase in the volume of FEV₁ in about 12 weeks, about 8 weeks, about 6 weeks, about 4 weeks, about 3 weeks, or about 2 weeks.

Embodiments described herein are directed to methods of treating the uncontrolled severe asthma of the eosinophilic phenotype in the subject comprising orally administering a daily dose of about 75 mg to about 300 mg of dexpramipexole, wherein FVC is improved. In certain embodiments, the uncontrolled asthmatic human subject had a baseline FVC of about 3.0 L to about 3.3 L, which is about a 74%-80% reduction from normal/predicted. In some embodiments, the FVC is increased by about 2% to about 12%. More preferably, the FVC is increased by about 8% to about 12%. In some embodiments, the volume of FVC is increased by about 20 ml to about 450 ml. More preferably, the volume of FVC is increased by about 200 ml to about 450 ml.

In some embodiments, treating the uncontrolled severe asthma of the eosinophilic phenotype in the subject by administration of a daily dose of about 75 mg to about 300 mg of dexpramipexole results in an increase in the volume of FVC in about 12 weeks, about 8 weeks, about 6 weeks, about 4 weeks, about 3 weeks, or about 2 weeks.

In some embodiments, treating the uncontrolled severe asthma of the eosinophilic phenotype in the subject by administration of a daily dose of about 75 mg to about 300 mg of dexpramipexole results in an increase in morning peak expiratory flow (PEF). Peak flow zones are areas of measurement on a peak flow meter. The goal of the peak flow zones is to show early symptoms of uncontrolled asthma. The green zone is 80% to 100% of the subject's highest peak flow reading. The yellow zone is 50% to 80% of the subject's highest peak flow reading. Measurements in this zone are a sign that large airways are starting to narrow and subject may start to have mild symptoms, such as coughing, feeling tired, feeling short of breath, or chest tightening. The red zone is less than 50% of the subject's highest peak flow reading. Readings in this zone mean severe narrowing of large airways, which is a medical emergency. In some embodiments, treating the uncontrolled severe asthma of the eosinophilic phenotype in the subject by administration of a daily dose of about 75 mg to about 300 mg of dexpramipexole results in the subject's ability to remain in the green zone when measuring morning peak expiratory flow.

Airway inflammation can also be assessed using the FeNO test, fractional concentration of exhaled nitric oxide, wherein the level of nitric oxide is measured in parts per billion (ppb) in the air you slowly exhale. Higher than normal levels of exhaled nitric oxide generally means the airways are inflamed, a sign of asthma. Levels less than about 20 ppb in children and less than about 25 ppb in adults are considered normal. Greater than about 35 ppb in children and 50 ppb in adults are considered to be abnormal.

Embodiments described herein are directed to methods of treating the uncontrolled severe asthma of the eosinophilic phenotype in the subject comprising orally administering a daily dose of about 75 mg to about 300 mg of dexpramipexole, wherein the uncontrolled severe asthma of the eosinophilic phenotype in the subject is controlled. The Asthma Control Questionnaire (ACQ), and/or the Asthma Control Test (ACT)™ can be used to assess whether asthma is being controlled. In certain embodiments, the uncontrolled asthmatic human subject had a baseline score of about 1.9 to about 2.3 on the ACQ and about 4.5 to about 5.4 on the AQLQ.

The ACQ cutoff for controlled asthma is a score of about 0.75, and the ACQ cutoff for uncontrolled asthma is a score of about 1.5. An ACQ score change of ≥0.5 is considered clinically significant. In embodiments described herein, the uncontrolled asthmatic human subject has a change in ACQ score of at least 0.5 points. In embodiments described herein, the uncontrolled asthmatic human subject has an improvement in score on ACQ and the score is less than or equal to 0.75.

Using the Asthma Control Test (ACT)™, a score of less than 19 indicates the individual's asthma may not be well controlled and a score of less than 15 indicates the individual's asthma may be very poorly controlled. In embodiments described herein, the uncontrolled asthmatic human subject has a change in ACT score of at least 0.5 points. In embodiments described herein, the uncontrolled asthmatic human subject has an improvement in score on Asthma Control Test (ACT)™ and the score is greater than 19.

The AQLQ is a 32-item asthma specific questionnaire designed to measure functional impairments that are most important to patients with asthma. The 32 questions in the AQLQ are divided into four domains; activity limitations, symptoms, emotional function, and environmental stimuli. Individual questions are equally weighted. The overall AQLQ score is the mean of the responses to each of the 32 questions and ranges from 1 to 7. A score 7.0 indicates that the patient has no impairments due to asthma and score 1.0 indicates severe impairment. Embodiments described herein are directed to methods of treating the uncontrolled severe asthma of the eosinophilic phenotype in the subject comprising orally administering a daily dose of about 75 mg to about 300 mg of dexpramipexole, wherein the subject's quality of life is improved. The Asthma Quality of Life Questionnaire (AQLQ) can be used to assess an individual's quality of life, scores range from 1-7, with higher scores indicating better quality of life. In embodiments described herein, the uncontrolled asthmatic human subject has a change in AQLQ score of at least 0.5 points. In embodiments described herein, the uncontrolled asthmatic human subject has an improvement in score on Asthma Quality of Life Questionnaire (AQLQ) and the score is greater than 3.5.

In embodiments described herein, the uncontrolled asthmatic human subject has an improvement in score on the St. George's Respiratory Questionnaire.

In some embodiments, treating the uncontrolled severe asthma of the eosinophilic phenotype in the subject by administration of a daily dose of about 75 mg to about 300 mg of dexpramipexole results in improving a subject's overall morbidity. In some embodiments, treating the uncontrolled severe asthma of the eosinophilic phenotype in the subject by administration of a daily dose of about 75 mg to about 300 mg of dexpramipexole results in improving a subject's overall mortality risk.

Dosage and Timing

In embodiments described herein, dexpramipexole or a pharmaceutically acceptable salt thereof, may be administered to the human subject at a daily dose of about 75 mg/day.

In embodiments described herein, dexpramipexole or a pharmaceutically acceptable salt thereof, may be administered to the human subject at a daily dose of about 150 mg/day.

In embodiments described herein, dexpramipexole or a pharmaceutically acceptable salt thereof, may be administered to the human subject at a daily dose of about 300 mg/day.

In embodiments described herein, dexpramipexole or a pharmaceutically acceptable salt thereof, may be administered to the human subject at a dose of about 37.5 mg twice per day.

In embodiments described herein, dexpramipexole or a pharmaceutically acceptable salt thereof, may be administered to the human subject at a dose of about 75 mg twice per day.

In embodiments described herein, dexpramipexole or a pharmaceutically acceptable salt thereof, may be administered to the human subject at a dose of about 300 mg twice per day.

In embodiments described herein, dexpramipexole is administered once daily for up to 24 weeks. In embodiments described herein, dexpramipexole is administered once daily for up to 12 weeks. In embodiments described herein, dexpramipexole is administered once daily for up to 8 weeks. In embodiments described herein, dexpramipexole is administered once daily for up to 6 weeks. In embodiments described herein, dexpramipexole is administered once daily for up to 4 weeks. In embodiments described herein, dexpramipexole is administered once daily until the moderate to severe asthma of the eosinophilic phenotype is resolved. In embodiments described herein, dexpramipexole is administered once daily for the lifetime of the subject.

In embodiments described herein, dexpramipexole is administered twice daily for up to 24 weeks. In embodiments described herein, dexpramipexole is administered twice daily for up to 12 weeks. In embodiments described herein, dexpramipexole is administered twice daily for up to 8 weeks. In embodiments described herein, dexpramipexole is administered twice daily for up to 6 weeks. In embodiments described herein, dexpramipexole is administered twice daily for up to 4 weeks. In embodiments described herein, dexpramipexole is administered twice daily until the moderate to severe asthma of the eosinophilic phenotype is resolved. In embodiments described herein, dexpramipexole is administered twice daily for the lifetime of the subject.

In some embodiments, the reduction of blood eosinophils is reduced within 1 week of treatment, within 2 weeks of treatment, within 3 weeks of treatment, within 4 weeks of treatment, within 5 weeks of treatment, within 6 weeks of treatment, within 7 weeks of treatment, within 8 weeks of treatment, within 9 weeks of treatment, within 10 weeks of treatment, within 11 weeks of treatment, or within 12 weeks of treatment. In some embodiments, the reduction of blood eosinophils is reduced wherein the subject is treated for 12 weeks.

Surprisingly, treatment with dexpramipexole provides a remittive effect. In certain embodiments, the moderate to severe asthma of the eosinophilic phenotype remains controlled after dexpramipexole has been discontinued for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In certain embodiments, the level of blood eosinophils remains reduced after dexpramipexole has been discontinued for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks.

In certain embodiments, the dexpramipexole is administered in combination with a bronchodilator selected from the group consisting of levalbuterol, ipratropium bromide, budesonide/formoterol, ipratropium, fluticasone/salmeterol, and combinations thereof. In certain embodiments, the dexpramipexole is administered in combination with an anti-inflammatory selected from the group consisting of zileuton, zafirlukast, and combinations thereof.

In certain embodiments, the dexpramipexole is administered instead of mepolizumab (NUCALA®). In certain embodiments, the dexpramipexole is administered instead of reslizumab (CINQAIR®). In certain embodiments, the dexpramipexole is administered instead of benralizumab (FASENRA®). In certain embodiments, the dexpramipexole is administered instead of dupilumab (DUPIXENT®). In certain embodiments, the dexpramipexole is administered instead of tezepelumab.

One of ordinary skill in the art will understand and appreciate the dosages and timing of the dosages to be administered to a human subject in need thereof. The doses and duration of treatment may vary, and may be based on assessment by one of ordinary skill in the art based on monitoring and measuring improvements in pulmonary and non-pulmonary tissues. This assessment may be made based on outward physical signs of improvement, such as decreased wheezing or less shortness of breath, or on internal physiological signs or markers. The doses may also depend on the condition or disease being treated, the degree of the condition or disease being treated and further on the age, weight, body mass index and body surface area of the subject.

In some embodiments, therapeutically effective amounts, daily doses, or single unit doses of dexpramipexole may be administered once per day or multiple times per day, such as 1 to 5 doses, twice per day or three times per day or every other day.

Embodiments are also directed to a dosage regimen for orally administering dexpramipexole or a pharmaceutically acceptable salt thereof to treat the conditions disclosed herein. For example, in some embodiments, the methods described herein may comprise a dosage regimen that may include a plurality of daily doses having an equal amount of dexpramipexole or a pharmaceutically acceptable salt thereof as the initial dose in one or more unit doses. In other embodiments, the dosage regimen may include an initial dose of dexpramipexole or a pharmaceutically acceptable salt thereof in one or more unit doses, then a plurality of daily doses having a lower amount of dexpramipexole or a pharmaceutically acceptable salt thereof as the initial dose in one or more unit doses. The dosage regimen may administer an initial dose followed by one or more maintenance doses. The plurality of doses following the administering of an initial dose may be maintenance doses.

Such embodiments are not limited by the amount of the initial dose and daily doses. For example, in particular embodiments, the initial dose and each of the plurality of daily doses may be from about 75 mg to about 300 mg of dexpramipexole. In preferred embodiments, the initial dose is about 37.5 mg twice per day or about 150 mg twice per day.

In some embodiments, two unit doses of about 37.5 mg are administered daily, wherein each unit dose may be substantially equal. In some embodiments, three unit doses of about 37.5 mg are administered daily, wherein each unit dose may be substantially equal.

In some embodiments, two unit doses of about 75 mg are administered daily, wherein each unit dose may be substantially equal. In some embodiments, three unit doses of about 75 mg are administered daily, wherein each unit dose may be substantially equal.

In some embodiments, two unit doses of about 150 mg are administered daily, wherein each unit dose may be substantially equal. In some embodiments, three unit doses of about 150 mg are administered daily, wherein each unit dose may be substantially equal.

Following the initial dose for a period of time, the maintenance dose may include administering less than the initial daily dose of 75 mg to about 300 mg administered twice per day as a 37.5 mg or 150 mg tablet, respectively, or administering the daily dose once per day. In preferred embodiments, the maintenance dose is 75 mg once per day or 300 mg once per day.

In further embodiments, the method may include an initial dosing regimen and a maintenance dosing regimen. In certain embodiments, the initial dosing regimen may include administering a higher dose of dexpramipexole or a pharmaceutically acceptable salt thereof than the maintenance dosing regimen as either a single administration or by administering an increased dosage for a limited period of time prior to beginning a maintenance dosing regimen of dexpramipexole or a pharmaceutically acceptable salt thereof. In some embodiments, subjects undergoing a maintenance regimen may be administered one or more higher-dosage treatments at one or more times during the maintenance dosage regimen.

In some embodiments, the initial dosing regimen and the maintenance dosing regimen may include administering dexpramipexole or a pharmaceutically acceptable salt thereof once per day, or twice per day. In such embodiments, the dosage regimen may continue administering an initial dose for 1, 2, 3, 4, 5, 6 or 7 days, up to 4 weeks, up to 8 weeks or up to 12 weeks. In some embodiments, the dosage regimen for administering an initial dose and/or a maintenance dose may continue for an extended period of time. Various embodiments are directed to a dosing regimen for dexpramipexole or a pharmaceutically acceptable salt thereof in which maintenance doses are maintained for an extended period of time without titration or otherwise changing the dosing regimen. In such embodiments, the extended period of time may be about 12 weeks or longer, about 6 months or longer, about 1 year or longer, 2, 3, 4, 5, or 10 years or longer, and in certain embodiments, an indefinite period of time.

In some embodiments, treatment with a daily dose of about 75 mg to about 300 mg of dexpramipexole is without the adverse side effects associated with dopamine agonism.

Oral pharmaceutical compositions containing dexpramipexole or a pharmaceutically acceptable salt thereof in a solid dosage may include, but are not limited to, softgels, tablets, capsules, cachets, pellets, pills, powders and granules; other oral dosage forms include, but are not limited to, solutions, suspensions, emulsions, and dry powder.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### EXAMPLES

### Example 1—Description of Dosage Form

The tablets for oral administration containing 150 mg of Dexpramipexole dihydrochloride monohydrate are formulated as described in Table A.

TABLE A
Composition of Dexpramipexole Tablets

| Component | Function | Quantity per tablet (mg) |
|---|---|---|
| Dexpramipexole | Drug Substance | 150.0 |
| dihydrochloride monohydrate | | |
| Microcrystalline cellulose/ | Diluent | 132.2 |
| Cellulose, microcrystalline | | |
| Mannitol | Diluent | 75.0 |
| Crospovidone | Disintegrant | 15.0 |
| Magnesium stearate | Glidant | 2.8 |

| | |
|---|---|
| Core Total | 375.0 |

Tablet Coat

| | | |
|---|---|---|
| Opadry II white | Film former | 11.3 |

| | |
|---|---|
| Coated tablet Total | 386.3 |

Summary of Tablets Containing 12.5, 20, 37.5, 75, 125, 150, and 200 mg of Dexpramipexole Dihydrochloride Monohydrate is provided in Table B. Tablets having 125 mg, 150 mg, or 200 mg comprise the same relative formulation, however the size of the tablet will be proportional to the dosage amount.

TABLE B
Formulations

| | Tablet Strength (mg) and Formulation (%) | | | | | |
|---|---|---|---|---|---|---|
| | 12.5 | 37.5 | 75 | 125 | 150 | 200 |
| Component | mg | mg | mg | mg | mg | mg |
| Dexpramipexole dihydrochloride | 4.00 | 12.00 | 24.00 | 40.00 | 40.00 | 40.00 |
| monohydrate (%) | | | | | | |
| Microcrystalline cellulose/Cellulose, | 58.22 | 53.11 | 45.46 | 35.25 | 35.25 | 35.25 |
| microcrystalline (%) | | | | | | |
| Mannitol (%) | 33.03 | 30.14 | 25.79 | 20.00 | 20.00 | 20.00 |
| Crospovidone (%) | 4.00 | 4.00 | 4.00 | 4.00 | 4.00 | 4.00 |
| Magnesium stearate (%) | 0.75 | 0.75 | 0.75 | 0.75 | 0.75 | 0.75 |
| Total (%) | 100 | 100 | 100 | 100 | 100 | 100 |

### Example 2—Phase 2 Clinical Study—KNS-760704-AS201 “A Randomized, Double-Blind, Placebo-Controlled Dose-Ranging Biomarker Study of the Effects of Dexpramipexole on Eosinophils in Subjects with Eosinophilic Asthma”

The primary objective of the clinical study was to evaluate the efficacy of dexpramipexole in reducing blood eosinophil count in subjects with eosinophilic asthma. The secondary objectives of the clinical study were to evaluate the safety and tolerability of dexpramipexole administered for 12 weeks in subjects with eosinophilic asthma, to evaluate the efficacy of dexpramipexole on pulmonary function, asthma control, and quality of life, and to evaluate the relative effect of dexpramipexole dosed at 75 mg/day, 150 mg/day, and 300 mg/day on blood eosinophil count. The clinical study also evaluated the efficacy of dexpramipexole in reducing tissue eosinophil biomarkers, evaluated the efficacy of dexpramipexole in reducing blood basophil count, evaluated the effect of dexpramipexole on blood eosinophil progenitor populations, evaluated the onset of blood eosinophil lowering, assess the recovery of blood eosinophil count after dexpramipexole discontinuation, evaluated the effect of dexpramipexole on asthma serum biomarkers, assessed the correlation between eosinophil lowering with changes in pulmonary function and asthma control, evaluated the exposure of dexpramipexole across the dose range used in the study, examined the relationship between dexpramipexole exposure and eosinophil-lowering response, and investigated potential predictive biomarkers to identify hematologic responders.

The endpoints used in the clinical study were as described in Table 1.

TABLE 1
Measurable Endpoints

| Primary Endpoint | Secondary Endpoints | Exploratory Endpoints |
|---|---|---|
| Change in blood absolute | Change in pre- | Change in pharyngeal and nasal eosinophil |
| eosinophil count from | bronchodilator FEV1, | peroxidase concentration from Baseline to |
| Baseline to Week 12 | from Baseline to | Week 12 |
| | Week 12 | |
| | Change in Asthma | |
| | Control Questionnaire | |
| | (ACQ-6) score, from | |
| | Baseline to Week 12 | |
| | Change in Asthma | Change in blood basophils, from Baseline to |
| | Control Questionnaire | Week 12 |
| | (ACQ-7) score, from | |
| | Baseline to Week 12 | |
| | Change in post- | Change in blood eosinophil progenitor |
| | bronchodilator FEV1, | populations, from Baseline to Week 12 |
| | from Baseline to | |
| | Week 12 | |
| | Change in quality of | Change in fractional exhaled nitric oxide |
| | life, as measured by the | (FeNO), from Baseline to Week 12 |
| | Asthma Quality of Life | |
| | Questionnaire (AQLQ), | |
| | from Baseline to | |
| | Week 12 | |
| | Incidence and severity | The fraction of hematologic responders, defined |
| | of AEs, changes in vital | as subjects with a ≥90% reduction of blood |
| | signs, clinical | eosinophil count, from Baseline to Week 12 |
| | laboratory safety tests, | |
| | physical examination, | |
| | body weight, and ECGs | |
| | | Kinetics of eosinophil response, defined at each |
| | | time point as the fraction of subjects having a |
| | | hematologic response at that time point or |
| | | previously, from Baseline to Week 12 |
| | | Kinetics of blood eosinophil recovery after |
| | | discontinuation of dexpramipexole during the |
| | | Eosinophil Recovery Period, defined as the |
| | | fraction of subjects with an AEC ≥50% of the |
| | | Baseline value over time, Week 12 to Week 24 |
| | | (hematologic responder population) |
| | | Changes in biomarkers (e.g., cytokines) in |
| | | peripheral blood: change from Baseline to |
| | | Week 12. Serum samples will be collected at |
| | | the Baseline, Week 12, and Week 24 visits for |
| | | storage and testing of samples for biomarker |
| | | evaluations of type 2 inflammation associated |
| | | mediators, including IL-5, IL-13, IL-33, ST2, |
| | | CCL2, CCL3, CCL4, CCL11, and CCL17. |
| | | Changes in urine eosinophil granule proteins, |
| | | from Baseline to Week 12 |
| | | Dexpramipexole plasma concentration across |
| | | the study dose range used in the study |
| | | Correlation between dexpramipexole exposure, |
| | | as measured by dexpramipexole |
| | | trough plasma concentration, and eosinophil- |
| | | lowering response |
| | | Whole genome DNA sequencing to discover |
| | | potential predictive genetic biomarkers to |
| | | identify hematologic responders |

The clinical study tested oral administration of dexpramipexole tablets BID for 12 weeks at 75 mg/day, 150 mg/day, and 300 mg/day, versus placebo. The clinical study tested approximately 100 subjects randomized approximately 1:1:1:1; placebo, dexpramipexole 75 mg/day, 150 mg/day, and 300 mg/day (25 per arm). Table 2 provides the summary of the subjects selected for the clinical study. The protocol enrolled symptomatic eosinophilic asthmatic subjects 18-75 years with moderate to greater disease severity as defined by GINA steps 3-5. Subjects must have had an FEV1 of <80% of predicted at Screening and Baseline and bronchodilator FEV1 reversibility after albuterol inhalation of ≥12% and ≥200 ml at Screening.

TABLE 2
Summary of Screening Demographics and Baseline Characteristics

| | | | 75 mg/day | | 150 mg/day | | 300 mg/day | |
|---|---|---|---|---|---|---|---|---|
| | Placebo | | dexpramipexole | | dexpramipexole | | dexpramipexole | |
| | (n = 27) | | (n = 22) | | (n = 26) | | (n = 28) | |
| Mean age | 45.8 | | 46.6 | | 44.5 | | 44.6 | |
| Sex: Male (%) | 10 | (37.0%) | 11 | (50.0%) | 12 | (46.2%) | 12 | (42.9%) |
| Sex: Female (%) | 17 | (63.0%) | 11 | (50.0%) | 14 | (53.8%) | 16 | (57.1%) |
| Race: White | 21 | (77.8%) | 17 | (77.3%) | 16 | (61.5%) | 22 | (78.6%) |
| Race: African | 4 | (14.8%) | 4 | (18.2%) | 6 | (23.1%) | 6 | (21.4%) |
| American | | | | | | | | |
| Race: Asia | 1 | (3.7%) | 1 | (4.5%) | 2 | (7.7%) | — | |
| Race: other | 1 | (3.7%) | — | | 2 | (7.6%) | — | |
| Mean BMI | 34.31 | | 31.73 | | 33.44 | | 32.13 | |
| Mean asthma | 30.21 | | 33.16 | | 29.08 | | 30.87 | |
| duration (yrs) | | | | | | | | |
| GINA Step 3* (%) | 10 | (37.0%) | 8 | (36.4%) | 4 | (15.4%) | 3 | (10.7%) |
| GINA Step 4* (%) | 14 | (51.9%) | 9 | (40.9%) | 15 | (57.7%) | 19 | (67.9%) |
| GINA Step 5* (%) | 3 | (11.1%) | 5 | (22.7%) | 7 | (26.9%) | 6 | (21.4%) |

| | | | | |
|---|---|---|---|---|
| Baseline FEV1 (L) | 2.05 | 1.98 | 2.03 | 2.11 |
| Baseline FEV1 | 62.7 | 59.0 | 63.6 | 62.3 |
| (% predicted) | | | | |

*As defined in the 2019 GINA guidelines.

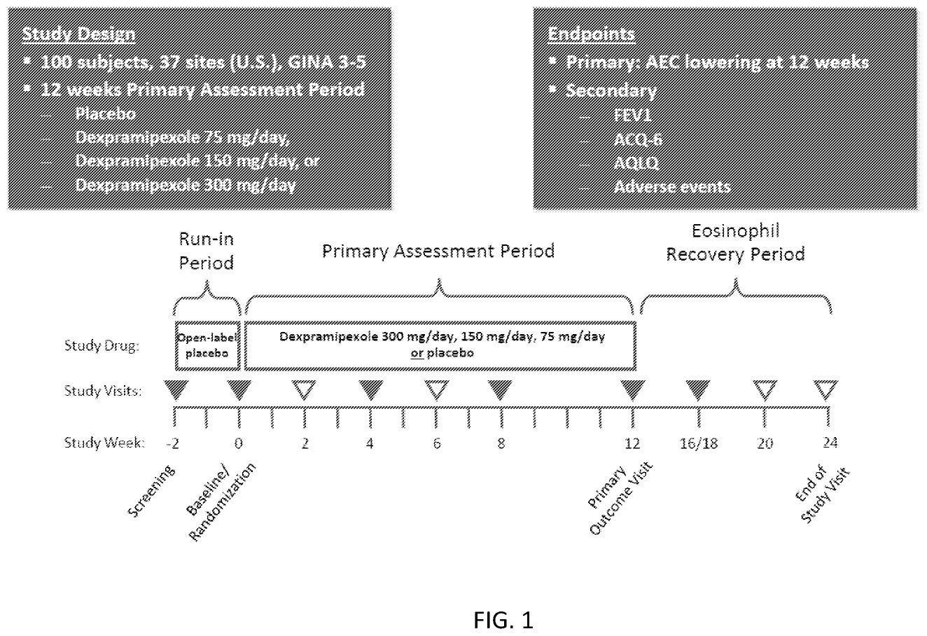

This clinical study is a randomized, double-blind, placebo-controlled, parallel-group, dose-ranging, multi-center study. Subjects received open-label placebo during the Run-in Period of the study, and subjects received double-blind study drug during the Primary Assessment Period of the study. See FIG. 1.

Duration of Treatment and Follow-up included: 1) Run-in Period: Screening through initiation of Baseline. Subjects underwent Screening assessments to assess whether they satisfied the eligibility criteria. They participated in the Run-in Period (of at least 12 days duration) to confirm a stable asthma medication regimen and to assess adherence to the dosing schedule, after which eligible subjects were randomized. 2) Primary Assessment Period: Baseline through completion of Week 12. After the collection of all Baseline assessments, subjects began receiving study drug for 12 weeks. Subjects had a site visit at Week 4, Week 8, and Week 12, and a CBC collected at Week 2 and Week 6 (on site or remotely). The Week 12 visit was the Primary Outcome Visit for the study. Subjects took the last dose of study drug at the Week 12 visit. 3) Eosinophil Recovery Period: Conclusion of Week 12 assessments through Week 24. Following the Primary Assessment Period, subjects entered the Eosinophil Recovery Period. During this period, subjects were seen at the site at either Week 16 or Week 18. Specific sites were designated to perform either Week 16 or Week 18 visits for subjects enrolled at their site. The Week 20 and Week 24 visits were at the site or at a designated remote laboratory facility.

Study Population: In this study, dexpramipexole was added to existing asthma therapy in subjects with Global Initiative for Asthma (GINA) steps 3-5 persistent asthma (requiring at minimum a low dose inhaled corticosteroid in combination with a long-acting β-agonist). The study population was limited to subjects with a physician diagnosis of asthma for ≥12 months and eligibility criteria described herein. In addition to its role in defining eosinophilic asthma, blood absolute eosinophil count (AEC) is also a biomarker associated with greater risk of asthma exacerbation. Additionally, elevated AEC is predictive of patients with greater clinical improvement to anti-eosinophil therapies. Accordingly, the eligibility criteria for this trial requires an AEC of ≥0.30×10⁹/L. This eligibility criterion was intended to select an asthmatic population comparable to the package label for approved eosinophil-lowering biologics.

Study Design: Approximately half of asthma patients fail to achieve adequate control of asthma symptoms and exacerbations when treated with approved asthma therapy, typically consisting of inhaled corticosteroids and long acting β-agonists. Although some of the responsible factors are modifiable, such as suboptimal medication adherence and poor inhaler technique, others reflect intrinsic deficiencies in available asthma pharmacotherapy. This deficiency is particularly apparent in patients prone to severe asthma exacerbations. Eosinophil-lowering biologics, such as mepolizumab, reslizumab, and benralizumab, have clearly demonstrated that significant lowering of blood and/or tissue eosinophils results in reduction of asthma exacerbations and improvement in asthma symptoms. However, all of the biologics require parenteral administration. Thus, there is a need and opportunity for an oral eosinophil-lowering drug, such as dexpramipexole, for the management of eosinophilic asthma. A 4-arm dose ranging placebo-controlled design was chosen to provide a range of drug exposure. Blood eosinophil lowering was chosen as the primary endpoint to facilitate identification of the lowest effective dose for use in future clinical development. The Primary Assessment Period of 12 weeks was chosen to provide adequate time to observe both pharmacodynamic endpoints, such as eosinophil lowering, as well as the clinical endpoints that may improve as a consequence of eosinophil lowering (FEV1, FVC, ACQ-6, ACQ-7, and AQLQ). The 12-week Eosinophil Recovery Period was chosen based on expected recovery to near baseline count in most subjects.

Patient inclusion/exclusion criteria. To be eligible to participate in this study, candidates must have met the following criteria:
1. Signed informed consent.
2. Male or female ≥18 and <75 years of age at the time of consent.
3. Willing to practice one highly effective method of contraception or 2 protocol acceptable methods of contraception in tandem, from the time of informed consent through 1 month (females) or 3 months (males) after the last dose of study drug.
4. Physician diagnosis of asthma for ≥12 months (relative to Baseline) based on Global Initiative for Asthma (GINA) 2018 Guidelines.
5. Asthma requiring treatment with, at a minimum, low dose inhaled corticosteroids in combination with a long-acting β2 agonist (GINA steps 3-5), on a stable dose for at least 1 month before Screening. Subjects using other controller options without long-acting β2 agonist are not eligible for the study.
6. Bronchodilator reversibility, as evidenced by ≥12% and ≥200 mL improvement in FEV1 15 to 25 minutes following inhalation of albuterol at Screening.
7. Pre-bronchodilator FEV1 ≥40% and <80% of predicted at Screening and Baseline.
8. AEC ≥0.30×10⁹/L at the Screening visit. May be repeated once if the initial value is between 0.25-0.29×10⁹/L; the second AEC must be ≥0.30×10⁹/L.
9. ACQ-7 ≥1.5 at Screening.
10. Negative pregnancy test at Baseline.
11. Adherence ≥85% with twice-daily placebo taken during the Run-in Period (minimum 12 days of adherence data), as documented by the smart bottle.

Exclusion Criteria. Candidates were excluded from study entry if any of the following had been documented by Baseline:
1. Asthma considered by the Site Investigator as unstable at Baseline.
2. Treatment for an asthma exacerbation within 8 weeks prior to Baseline visit.
3. Current diagnosis of allergic bronchopulmonary aspergillosis, eosinophilic granulomatosis with polyangiitis, eosinophilic gastrointestinal diseases, or hypereosinophilic syndrome, or lung diseases (e.g., chronic obstructive pulmonary disease, idiopathic pulmonary fibrosis) which may confound interpretation of this trial's findings.
4. Infection of the upper or lower respiratory tract, including paranasal sinuses and middle ear within the 4 weeks prior to Baseline.
5. Treatment with systemic corticosteroids in the 8 weeks prior to Screening.
6. Treatment with an investigational drug in the previous 30 days or 5-half-lives prior to Baseline, whichever is longer.
7. Treatment with monoclonal antibody therapy, including benralizumab, dupilumab, mepolizumab, reslizumab, omalizumab, or TNF inhibitors, within 5-half-lives prior to Baseline.
8. Treatment with pramipexole within 4 weeks of Baseline.
9. Treatment with selected drugs known to have a substantial risk of neutropenia.
10. Planned surgical procedures during the conduct of the study.
11. History of malignancy. Subjects with basal cell carcinoma, localized squamous cell carcinoma of the skin, or in-situ carcinoma of the cervix are not excluded, provided that the subject is in remission and curative therapy was completed ≥12 months prior to Screening. Subjects with other malignancies are not excluded, provided that the subject is in remission and curative therapy was completed ≥5 years prior to Screening.
12. Known history of human immunodeficiency virus (HIV) infection.
13. Active hepatitis B or C infection. Subjects with a history of hepatitis C with undetectable viral load for ≥1 year are not excluded.
14. Renal dysfunction, defined as an estimated glomerular filtration rate (eGFR)<60 mL/min/1.73m2 at Screening (using the Chronic Kidney Disease Epidemiology Collaboration [CKD-EPI] formula).
15. History of unstable or severe cardiac, hepatic, or renal disease, or other medically significant illness.
16. Medical or other condition likely to interfere with subject's ability to undergo study assessments, adhere to visit schedule, or comply with study requirements.
17. Helminth infection within 6 months prior to Baseline.
18. Use of any smoke or inhaled nicotine delivery device within 1 year prior to Screening or a smoking history ≥10 pack-years.
19. Known or suspected alcohol or other substance abuse.
20. Known or suspected non-adherence with study dosing schedule.
21. Unwillingness or inability to follow the procedures outlined in the protocol, including throat or nasal swab.
22. Absolute neutrophil count <2.0×10⁹/L at Screening, or any documented history of absolute neutrophil count <2.0×10⁹/L.
23. History of long QT syndrome or arrhythmia.
24. ECG showing prolongation of QTc interval calculated using Fridericia's heart rate correction formula (QTcF) >450 ms at the Screening visit or pre-dose at Baseline. QTcF interval calculated as the mean of triplicate determinations.
25. Clinically important abnormalities in resting ECG at Screening or Baseline, including any of the following:
   a) PR interval >210 ms;
   b) QRS>110 ms;
   c) Heart rate <45 bpm or >100 bpm (average of 3 assessments).
26. Pregnant or breastfeeding women.

Concomitant Therapy. A concomitant therapy is any drug or substance administered between the Screening visit and the subject's last study visit. All concomitant medication was recorded in the subject's source document and on the case report form (CRF).

Concomitant Therapy Restrictions. Inhaled corticosteroids: The dose of any inhaled or intranasal corticosteroids was stabilized for at least 4 weeks prior to the first dose of study drug and remained constant throughout the study. Systemic corticosteroids: Medically indicated use of systemic corticosteroids to treat an asthma exacerbation occurring while on study were allowed on the study. Investigational drugs: Use of investigational drugs was prohibited for the duration of the study. Monoclonal antibody therapy: The use of monoclonal antibody therapy, including benralizumab, dupilumab, mepolizumab, reslizumab, omalizumab, or TNF inhibitors was prohibited throughout the duration of the study. Pramipexole: The use of pramipexole was prohibited throughout the duration of the study. Selected drugs known to have substantial risk of neutropenia: Drugs known to have a substantial risk of neutropenia were prohibited for the duration of the study.

Evaluation of Safety: Physical examination, vital signs (systolic and diastolic blood pressure, respiratory rate, heart rate, and temperature), body weight, 12-lead ECG, clinical laboratory evaluations (hematology, blood chemistry, and urinalysis), pregnancy testing, adverse event monitoring, and concomitant medication were monitored throughout the study. As neutropenia is an adverse event of special interest for dexpramipexole, neutrophil counts were reported using a central laboratory and monitored by the study medical monitor. A neutropenia case report form was utilized during the study to capture detailed clinical information concurrent with any laboratory events of neutropenia. To assess potential cardiac drug interactions of albuterol and dexpramipexole, Week 8 ECGs were timed to correspond with approximate Cₘₐₓ dexpramipexole plasma concentrations.

Evaluation of Efficacy: Blood eosinophil count, changes in pulmonary function (FEV1), Asthma Control Questionnaire (ACQ-6), Asthma Control Questionnaire (ACQ-7), Asthma Quality of Life Questionnaire (AQLQ), exhaled nitric oxide, pharyngeal and nasal eosinophil peroxidase were collected periodically during the study.

Pharmacokinetic Evaluations: Blood for plasma drug concentration was collected at the Week 4 (trough), Week 8 (trough and post-dose at 2 hours), and Week 12 (trough) visits. Dexpramipexole trough plasma concentration was used to evaluate the relationship between dexpramipexole exposure and eosinophil-lowering response.

Statistics: The study used a blocked randomization stratified by study site. The safety sample population was used for all safety analyses and the modified ITT population was used for efficacy analyses. The modified ITT population, which consists of all subjects who received at least one dose of study drug and who have at least one post-randomization evaluation for at least one of the efficacy endpoints, was used for efficacy analyses.

The primary endpoint of this study was the change in blood absolute eosinophil count from Baseline to Week 12. Absolute eosinophil counts were transformed to the log 10 scale. To avoid taking the log of zero, zeroes were replaced with 5 cells/μL in conventional units and 0.005×10⁹/L in the International System of Units (SI), which is 50% of the lower limit of quantification. The geometric mean of all eosinophil counts obtained between the Screening and Baseline visits were used to establish the baseline eosinophil count used in the efficacy analyses. Geometric means and standard deviations were presented by treatment group for observed values at each visit along with a p-value comparing each dexpramipexole treatment group to placebo based on an analysis of variance (ANOVA).

The primary analysis was a mixed-effect model with repeated-measures (MMRM) with terms for baseline, the 3 level of GINA treatment step as a categorical variable, treatment, visit, treatment by visit interaction, and baseline by visit interaction as fixed effects, and subjects as a random effect. An unstructured covariance was used.

Dexpramipexole group treatment effects and treatment group effects versus placebo at Week 12 was tested by contrasts within the MMRM. Estimated LS means of treatment effects and estimated difference in treatment effects at each visit were back transformed to the original scale to present estimated geometric means for treatment effects and ratio of geometric means of treatment effects along with 95% CI.

A contrast was created to test the treatment effect at Week 12 for the pooled 150 mg/day and 300 mg/day group versus the placebo group. A contrast was created to test the treatment effect at Week 12 for log-linear dose response.

To control the alpha level for testing 3 dose groups versus placebo for the primary endpoint and for testing FEV1, a closed hierarchical testing procedure was used in the following order: 1. First, the 300 mg/day dose group was tested versus placebo for change in blood absolute eosinophil count, and if this reached the <0.05 level then, 2. the 150 mg/day dose group was tested versus placebo at the 0.05 level for change in blood absolute eosinophil count, and if this reached the <0.05 level then, 3. the pooled 150 and 300 mg/day dose groups were tested versus placebo at the 0.05 level for change in FEV1 at Week 12, then 4. the 75 mg/day dose group was tested versus placebo at the 0.05 level for change in blood absolute eosinophil count, and if this reached the <0.05 level.

An interim analysis of efficacy after all subjects have completed the Week 12 visit (Primary Outcome Visit) was performed. There were no p-value adjustment for the interim analysis because subjects were off study drug after the Week 12 visit and they completed the assessment of the primary endpoint. The primary purpose of visits after Week 12 was to observe eosinophil recovery following discontinuation of study drug and to monitor subjects for safety.

Sample Size: The primary endpoint for this study was change in blood absolute eosinophil count from Baseline to Week 12. In an open label study of dexpramipexole in subjects with chronic rhinosinusitis with nasal polyps (KNS-760704-CS201), dexpramipexole reduced eosinophils by 94% (ratio of endpoint to baseline=−2.81 on the log base e scale). The standard deviation for the ratio of endpoint to baseline was 1.82 on the log base e scale.

Nineteen subjects per arm provided approximately 84% power if the true reduction in blood eosinophils was 85% with dexpramipexole and 10% with placebo. The power was 95% if the true reduction in blood eosinophils was 90% with dexpramipexole and 10% with placebo. The sample size was calculated using methodology for a two-sample t-test. Assuming an approximate 20% dropout rate of subjects who do not have the final observation for blood eosinophils yields a sample size of 25 subjects randomized per arm.

Results of the clinical study demonstrated that the eosinophil lowering primary endpoint was highly significant and that this highly significant dose-response trend had a significant effect on absolute eosinophil count (AEC) at all dose levels. The highly significant dose-dependent response lead to an increase hematologic responders, defined as subjects with ≥505 reduction in AEC or a lowering of AEC to <100 cells/μL. The increase in FEV1 highly correlated with the reduction in eosinophil count and the hematologic responders had clinically important and statistically significant improvement in lung function measured by FEV1.

For completeness, Table 3 provides the final number of subjects who completed the study per study arm.

TABLE 3
Summary of Subject Disposition and Study Assignment (Population: All Subjects)

| | | | 75 mg/day | | 150 mg/day | | 300 mg/day | | | |
|---|---|---|---|---|---|---|---|---|---|---|
| | Placebo | | dexpramipexole | | dexpramipexole | | dexpramipexole | | Overall | |
| | (N = 27) | | (N = 22) | | (N = 26) | | (N = 28) | | (N = 103) | |
| | n (%) | | n (%) | | n (%) | | n (%) | | n (%) | |
| Completed | 25 | (92.6%) | 22 | (100%) | 24 | (92.3%) | 28 | (100%) | 99 | (96.1%) |

| the Primary | | | | | | | | |
|---|---|---|---|---|---|---|---|---|
| Assessment | | | | | | | | |
| Phase | | | | | | | | |
| Discontinued | 2 | (7.4%) | — | 2 | (7.7%) | — | 4 | (3.9%) |
| the Primary | | | | | | | | |
| Assessment | | | | | | | | |
| Phase | | | | | | | | |
| Adverse | 1 | (3.7%) | — | — | | — | 1 | (1.0%) |
| Event | | | | | | | | |
| Withdrawal | 1 | (3.7%) | — | 1 | (3.8%) | — | 2 | (1.9%) |

| by Subject | | | | | | | |
|---|---|---|---|---|---|---|---|
| Physician | — | — | 1 | (3.8%) | — | 1 | (1.0%) |

Decision

n = number of subjects with valid observations, N = number of subjects, %-percentage of subjects with valid observations (n/N × 100)

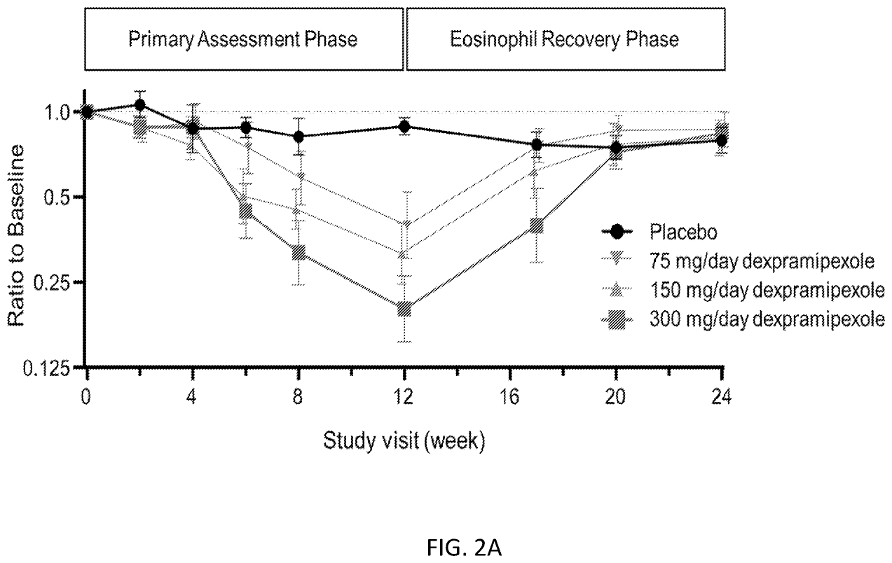

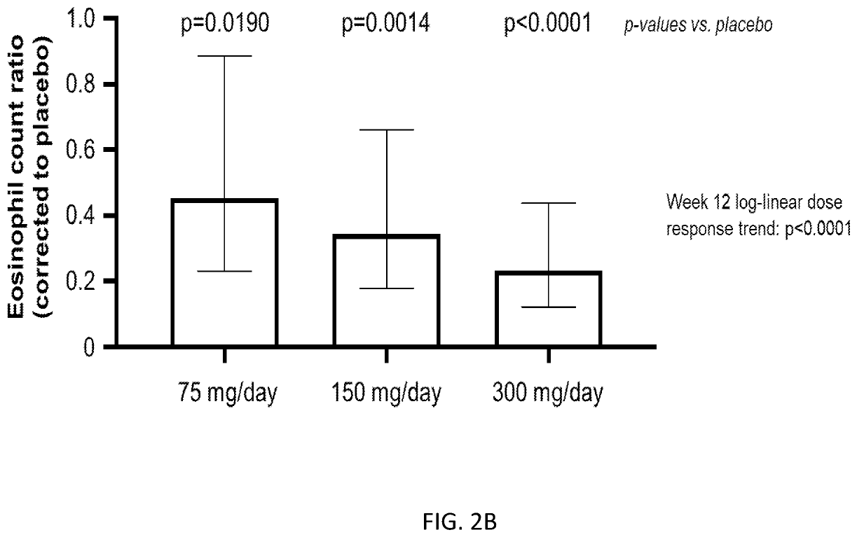

The primary endpoint of the study was met: a highly significant decrease in eosinophil count in blood compared with baseline at week 12 was observed. As can be seen in Table 4, up to an 80% reduction in AEC was observed. FIGS. 2A and 2B graphically depicts the lowering of AEC in a dose-dependent manner up to week 12 and continues following dexpramipexole discontinuation at week 12, during the subsequent return to baseline AEC in weeks 13-24.

TABLE 4
Primary Analysis of Blood Absolute Eosinophil Counts
During the Primary Assessment Phase (Population: Efficacy)

| | | | | | Ratio vs | |
|---|---|---|---|---|---|---|
| | | | Analysis | Ratio to | Placebo | |
| | | | Baseline | baseline | geometric | P-value |
| | | | (cells/ | geometric | LS mean | vs |
| Timepoint | Treatment | n | μL) | LS mean | (95% CI) | Placebo |
| Week 12 | Placebo | 25 | 382 | 0.8980 | | |
| (Primary | 75 mg/day | 22 | 404 | 0.4031 | 0.4489 | 0.0190 |
| Endpoint) | dexpram- | | | | (0.231, | |
| | ipexole | | | | 0.874) | |
| | 150 mg/day | 24 | 374 | 0.3056 | 0.3403 | 0.0014 |
| | dexpram- | | | | (0.177, | |
| | ipexole | | | | 0.653) | |
| | 300 mg/day | 28 | 438 | 0.2051 | 0.2283 | <0.0001 |
| | dexpram- | | | | (0.121, | |
| | ipexole | | | | 0.431) | |
| Week 12 | | | | | | <0.0001 |
| log-linear | | | | | | |
| dose | | | | | | |
| response | | | | | | |
| trend | | | | | | |

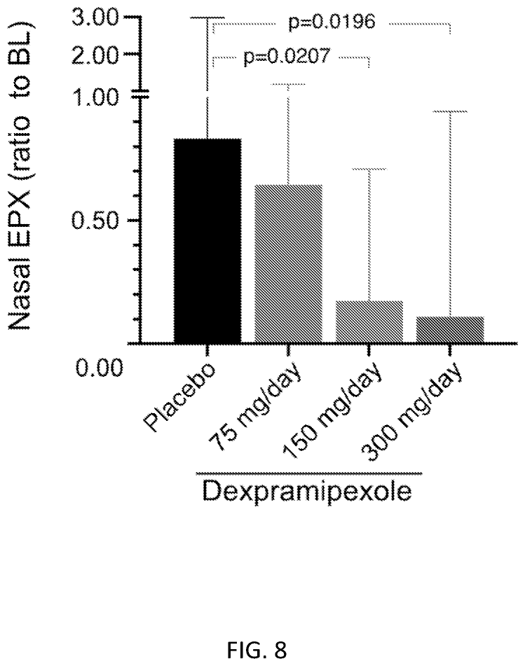

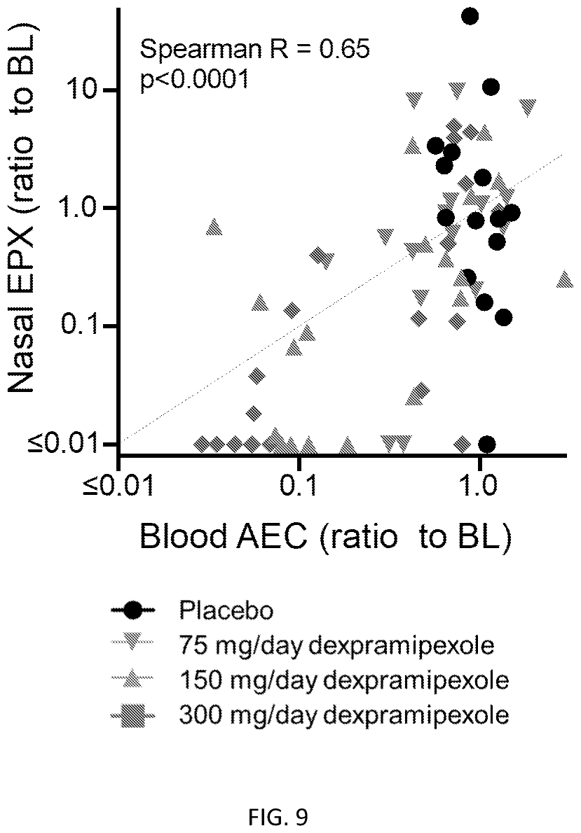

As compared with placebo, significant lowering in the ration of nasal tissue EPX to baseline by dexpramipexole is observed at week 12 in both the 150 mg/day and the 300 mg/day dexpramipexole dose groups (FIG. 8). Further, FIG. 9 demonstrated the strong correlation with the reduction of nasal tissue eosinophil peroxidase (EPX) and reduction in blood absolute eosinophil count (AEC).

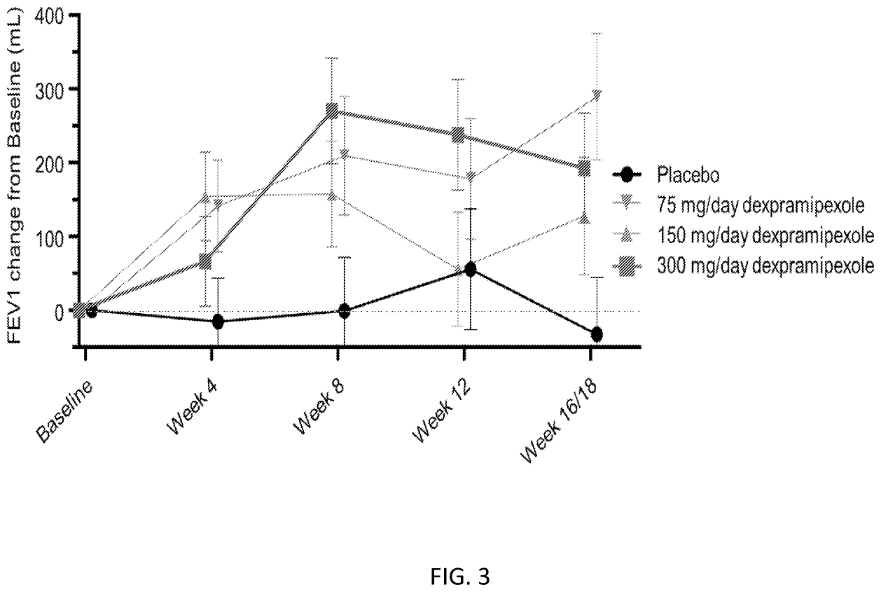

Table 5 and FIG. 3 present significantly improved FEV1 data from the clinical study for each of the 3 dosage groups compared with placebo.

TABLE 5
FEV1 Data

| | | | Change | Difference | |
|---|---|---|---|---|---|
| | | | from | versus | |
| | | | Baseline | Placebo (L) | P-value |
| | | | (L) | LSM (95% CI) | VS |
| Timepoint | Treatment | n | LSM | [SE] | Placebo |
| Analysis | Placebo | 26 | | | |
| Baseline | 75 mg/day | 22 | | | |
| | dexpramipexole | | | | |
| | 150 mg/day | 25 | | | |
| | dexpramipexole | | | | |
| | 300 mg/day | 28 | | | |
| | dexpramipexole | | | | |
| Week 4 | Placebo | 23 | −0.0153 | | |
| | 75 mg/day | 20 | 0.150 | 0.165 | 0.0548 |
| | dexpramipexole | | | (−0.00345, 0.334) | |
| | | | | [0.0848] | |
| | 150 mg/day | 22 | 0.151 | 0.166 | 0.0502 |
| | dexpramipexole | | | (−.000183, 0.333) | |
| | | | | [0.0836] | |
| | 300 mg/day | 23 | 0.0685 | 0.0838 | 0.3161 |
| | dexpramipexole | | | (−0.0815, 0.249) | |
| | | | | [0.0831] | |
| Week 8 | Placebo | 20 | −0.00645 | | |
| | 75 mg/day | 15 | 0.204 | 0.210 | 0.0521 |
| | dexpramipexole | | | (−0.00197, 0.423) | |
| | | | | [0.107] | |
| | 150 mg/day | 21 | 0.166 | 0.173 | 0.0930 |
| | dexpramipexole | | | (−0.0294, 0.374) | |
| | | | | [0.102] | |
| | 300 mg/day | 22 | 0.248 | 0.254 | 0.0130 |
| | dexpramipexole | | | (0.0550, 0.453) | |
| | | | | [0.100] | |
| Week 12 | Placebo | 17 | 0.0470 | | |
| | 75 mg/day | 18 | 0.168 | 0.121 | 0.2942 |
| | dexpramipexole | | | (−0.107, 0.349) | |
| | | | | [0.115] | |
| | 150 mg/day | 21 | 0.0601 | 0.0131 | 0.9071 |
| | dexpramipexole | | | (−0.209, 0.235) | |
| | | | | [0.112] | |
| | 300 mg/day | 24 | 0.209 | 0.162 | 0.1406 |
| | dexpramipexole | | | (−0.0546, 0.379) | |
| | | | | [0.109] | |
| Week 16 or | Placebo | 22 | −0.0328 | | |
| Week 18 | 75 mg/day | 17 | 0.295 | 0.327 | 0.0043 |
| | dexpramipexole | | | (0.105, 0.550) | |
| | | | | [0.112] | |
| | 150 mg/day | 20 | 0.128 | 0.161 | 0.1411 |
| | dexpramipexole | | | (−0.0544, 0.377) | |
| | | | | [0.109] | |
| | 300 mg/day | 25 | 0.207 | 0.240 | 0.0242 |
| | dexpramipexole | | | (0.0320, 0.448) | |
| | | | | [0.105] | |

Data on lung function in the 300 mg/day dexpramipexole group is provided in Tables 6A-6E as pre-bronchodilator FEV1 improvement versus placebo observed at all time points. This demonstrates that the pharmacodynamics effect of dexpramipexole on FEV1 is persistent and observed follow drug discontinuation.

TABLE 6A
Population: Efficacy: Pre-Bronchodilator FEV1 (L), % change from Baseline

| | | | 75 mg/day | 150 mg/day | 300 mg/day |
|---|---|---|---|---|---|
| | | Placebo | dexpramipexole | dexpramipexole | dexpramipexole |
| | | (N = 27) | (N = 22) | (N = 25) | (N = 28) |
| Timepoint | Statistic | % Change | % Change | % Change | % Change |
| Week 4 | Mean | 0.0654 | 6.25 | 7.31 | 1.42 |
| | n | 23 | 20 | 22 | 23 |
| Week 8 | Mean | 1.92 | 12.0 | 7.57 | 15.5 |
| | n | 20 | 15 | 21 | 22 |
| Week 12 | Mean | 3.63 | 9.27 | 3.14 | 12.5 |
| | n | 17 | 18 | 21 | 24 |
| Week 16 or | Mean | 0.110 | 17.6 | 7.71 | 10.8 |
| Week 18 | n | 22 | 17 | 20 | 25 |

TABLE 6B
Pre-Bronchodilator FEV1 (L)

| | | | Observed | Observed | Observed |
|---|---|---|---|---|---|
| | | Observed | 75 mg/day | 150 mg/day | 300 mg/day |
| | | Placebo | dex | dex | dex |
| Timepoint | Statistic | (N = 27) | (N = 22) | (N = 25) | (N = 28) |
| Screening | Mean | 1.98 | 2.03 | 1.93 | 2.06 |
| | SE | 0.0905 | 0.135 | 0.115 | 0.0933 |
| | Median | 1.91 | 2.06 | 1.86 | 2.10 |
| | Min | 1.22 | 1.02 | 0.970 | 1.14 |
| | Max | 2.93 | 3.46 | 3.60 | 2.95 |
| | n | 26 | 22 | 25 | 28 |
| Baseline Visit | Mean | 2.06 | 1.99 | 2.03 | 2.12 |
| | SE | 0.117 | 0.132 | 0.115 | 0.0989 |
| | Median | 1.89 | 1.91 | 2.00 | 1.98 |
| | Min | 1.23 | 1.17 | 1.08 | 1.35 |
| | Max | 3.50 | 3.88 | 3.26 | 3.04 |
| | n | 24 | 20 | 24 | 27 |

TABLE 6C
Pre-Bronchodilator FEV1 (L)

| | | Placebo (N = 27) | | | 75 mg/day dexpramipexole (N = 22) | | |
|---|---|---|---|---|---|---|---|
| | | | | % | | | % |
| Tinepoint | Statistic | Observed | Change | Change | Observed | Change | Change |
| Analysis | Mean | 2.05 | | | 1.98 | | |
| Base- | SE | 0.111 | | | 0.123 | | |
| line* | Median | 1.89 | | | 1.91 | | |
| | Min | 1.23 | | | 1.17 | | |
| | Max | 3.50 | | | 3.88 | | |
| | n | 26 | | | 22 | | |
| Week 4 | Mean | 2.08 | −.000435 | 0.0654 | 2.10 | 0.130 | 6.25 |
| | SE | 0.125 | 0.0412 | 1.90 | 0.156 | 0.0658 | 2.98 |
| | Median | 1.89 | −0.0100 | −0.680 | 2.10 | 0.0750 | 4.85 |
| | Min | 1.19 | −0.440 | −17.0 | 1.22 | −0.240 | −12.6 |
| | Max | 3.42 | 0.590 | 29.2 | 3.78 | 1.14 | 46.5 |
| | n | 23 | 23 | 23 | 20 | 20 | 20 |

| | | 150 mg/day dexpramipexole (N = 25) | | |
|---|---|---|---|---|
| | | Observed | Change | % Change |
| Analysis | Mean | 2.03 | | |
| Base- | SE | 0.111 | | |
| line* | Median | 2.02 | | |
| | Min | 1.08 | | |
| | Max | 3.26 | | |
| | n | 25 | | |
| Week | Mean | 2.15 | 0.123 | 7.31 |
| 4 | SE | 0.126 | 0.0642 | 3.40 |
| | Median | 2.05 | 0.0800 | 4.54 |
| | Min | 1.14 | −0.400 | −16.2 |
| | Max | 3.65 | 0.960 | 47.5 |
| | n | 22 | 22 | 22 |
| | | 300 mg/day dexpramipexole (N = 28) | | |
| | | Observed | Change | % Change |
| Analysis | Mean | 2.11 | | |
| Base- | SE | 0.0957 | | |
| line* | Median | 1.97 | | |
| | Min | 1.35 | | |
| | Max | 3.04 | | |
| | n | 28 | | |
| Week | Mean | 2.14 | 0.0417 | 1.42 |
| 4 | SE | 0.136 | 0.0608 | 3.03 |
| | Median | 2.16 | 0.0700 | 3.95 |
| | Min | 1.01 | −0.710 | −41.3 |
| | Max | 3.48 | 0.520 | 22.9 |
| | n | 23 | 23 | 23 |

TABLE 6D
Pre-Bronchodilator FEV1 (L)

| | | Placebo (N = 27) | | | 75 mg/day dexpramipexole (N = 22) | | |
|---|---|---|---|---|---|---|---|
| | | | | % | | | % |
| Tinepoint | Statistic | Observed | Change | Change | Observed | Change | Change |
| Analysis | Mean | 2.09 | 0.0360 | 192 | 228 | 0.237 | 12.0 |
| Base- | SE | 0.129 | 0.0438 | 2.10 | 0.187 | 0.0833 | 4.39 |
| line* | Median | 2.00 | 0.0150 | 0.863 | 2.24 | 0.170 | 10.7 |
| | Min | 1.38 | −0.440 | −17.5 | 1.02 | −0.470 | −31.5 |
| | Max | 3.58 | 0.440 | 20.4 | 3.86 | 0.900 | 36.7 |
| | n | 20 | 20 | 20 | 15 | 15 | 15 |
| Week 4 | Mean | 2.13 | 0.0706 | 3.63 | 2.24 | 0.207 | 9.27 |
| | SE | 0.129 | 0.0435 | 2.02 | 0.183 | 0.0868 | 4.21 |
| | Median | 1.95 | 0.0500 | 2.78 | 2.08 | 0.115 | 5.48 |
| | Min | 1.44 | −0.200 | −9.90 | 0.980 | −0.330 | −25.2 |
| | Max | 3.31 | 0.420 | 19.4 | 3.94 | 1.28 | 52.2 |
| | n | 18 | 17 | 17 | 18 | 18 | 18 |

| | | 150 mg/day dexpramipexole (N = 25) | | |
|---|---|---|---|---|
| | | Observed | Change | % Change |
| Analysis | Mean | 2.21 | 0.130 | 7.57 |
| Base- | SE | 0.130 | 0.0847 | 4.11 |
| line* | Median | 2.22 | 0.0900 | 3.88 |
| | Min | 1.30 | −0.400 | −18.8 |
| | Max | 3.28 | 1.19 | 58.9 |
| | n | 21 | 21 | 21 |
| Week | Mean | 2.12 | 0.0414 | 3.14 |
| 4 | SE | 0.132 | 0.0965 | 4.32 |
| | Median | 2.11 | −0.0400 | −1.72 |
| | Min | 0.960 | −0.920 | −30.5 |
| | Max | 3.19 | 1.06 | 52.5 |
| | n | 21 | 21 | 21 |
| | | 300 mg/day dexpramipexole (N = 28) | | |
| | | Observed | Change | % Change |
| Analysis | Mean | 2.32 | 0.277 | 15.5 |
| Base- | SE | 0.116 | 0.0851 | 4.45 |
| line* | Median | 2.40 | 0.250 | 10.5 |
| | Min | 1.38 | −0.470 | −17.3 |
| | Max | 3.29 | 1.35 | 70.3 |
| | n | 22 | 22 | 22 |
| Week | Mean | 2.29 | 0.236 | 12.5 |
| 4 | SE | 0.119 | 0.0768 | 4.18 |
| | Median | 2.45 | 0.210 | 10.0 |
| | Min | 1.23 | −0.320 | −18.6 |
| | Max | 3.38 | 1.19 | 64.3 |
| | n | 24 | 24 | 24 |

TABLE 6E
Pre-Bronchodilator Percent Predicted FEV1 (%)

| | | Placebo (N = 27) | | | 75 mg/day dexpramiperole (N = 22) | | |
|---|---|---|---|---|---|---|---|
| | | | | % | | | % |
| Tinepoint | Statistic | Observed | Change | Change | Observed | Change | Change |
| Analysis | Mean | 62.7 | | | 59.0 | | |
| Base- | SE | 1.48 | | | 2.31 | | |
| line* | Median | 64.0 | | | 57.8 | | |
| | Min | 46.1 | | | 43.8 | | |
| | Max | 78.7 | | | 78.2 | | |
| | n | 26 | | | 22 | | |
| Week 4 | Mean | 63.2 | 0.114 | 0.0641 | 62.2 | 3.59 | 62.4 |
| | SE | 2.11 | 1.15 | 1.90 | 2.75 | 1.58 | 2.98 |
| | Median | 64.3 | −0.380 | −0.690 | 64.4 | 2.69 | 4.85 |
| | Min | 44.6 | −9.78 | −17.0 | 40.3 | −6.02 | −12.6 |
| | Max | 82.6 | 16.6 | 29.2 | 81.1 | 23.0 | 46.5 |
| | n | 23 | 23 | 23 | 20 | 20 | 20 |

| | | 150 mg/day dexpramiperole (N = 25) | | |
|---|---|---|---|---|
| | | Observed | Change | % Change |
| Analysis | Mean | 63.6 | | |
| Base- | SE | 1.90 | | |
| line* | Median | 64.9 | | |
| | Min | 41.7 | | |
| | Max | 79.9 | | |
| | n | 25 | | |
| Week | Mean | 67.4 | 3.95 | 7.31 |
| 4 | SE | 2.22 | 1.99 | 3.41 |
| | Median | 69.2 | 2.85 | 4.54 |
| | Min | 45.4 | −9.82 | −16.2 |
| | Max | 83.0 | 26.7 | 47.5 |
| | n | 22 | 22 | 22 |
| | | 300 mg/day dexpramiperole (N = 28) | | |
| | | Observed | Change | % Change |
| Analysis | Mean | 62.3 | | |
| Base- | SE | 1.95 | | |
| line* | Median | 64.8 | | |
| | Min | 41.0 | | |
| | Max | 78.9 | | |
| | n | 28 | | |
| Week | Mean | 62.5 | 0.607 | 1.42 |
| 4 | SE | 2.62 | 1.81 | 3.03 |
| | Median | 62.3 | 2.34 | 3.94 |
| | Min | 32.2 | −22.6 | −41.3 |
| | Max | 83.1 | 14.5 | 22.9 |
| | n | 23 | 23 | 23 |

Pooled analysis of the FEV₁ data provides evidence in the magnitude of the improvements seen in subjects treated with dexpramipexole, see Table 7.

TABLE 7
Pooled data

| | | Pooled | Pooled | ΔFEV1 vs | | |
|---|---|---|---|---|---|---|
| | | Data | Data | Placebo | | P-value |
| Study | | points (n) | points (n) | (mL) LSM | | vs |
| arm(s) | Visit(s) | Placebo | Dex | (SEM) | | Placebo |
| 75 mg/day | Weeks 4, 8, | 82 | 70 | 206 | (86.3) | 0.0190 |
| | 12, & 16/18 | | | | | |
| | pooled | | | | | |
| 150 mg/day | Weeks 4, 8, | 82 | 84 | 126 | (84.4) | 0.1322 |
| | 12, & 16/18 | | | | | |
| | pooled | | | | | |
| 300 mg/day | Weeks 4, 8, | 82 | 94 | 185 | (83.0) | 0.0282 |
| | 12, & 16/18 | | | | | |
| | pooled | | | | | |
| All Dex | Week 4 | 23 | 65 | 138 | (68.0) | 0.0450 |
| arms pooled | | | | | | |
| All Dex | Week 8 | 20 | 58 | 212 | (83.5) | 0.0128 |
| arms | | | | | | |
| pooled @ | | | | | | |
| All Dex | Week 12 | 17 | 63 | 99 | (92.5) | 0.288 |
| arms | | | | | | |
| pooled @ | | | | | | |
| All Dex | Week 16/18 | 22 | 62 | 233 | (87.9) | 0.0069 |
| arms | | | | | | |
| pooled @ | | | | | | |
| All Dex | Weeks 4, 8, | 82 | 248 | 173 | (68.8) | 0.0136 |

| | |
|---|---|
| arms | 12, & 16/18 |
| pooled @ | pooled |

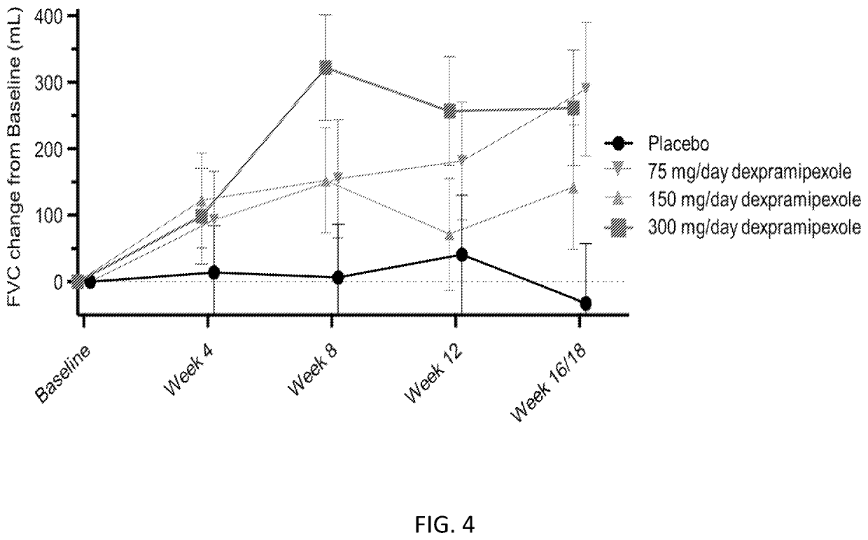

Significant improvement in lung function is also observed in the increase in volume of FVC. Data on pre-bronchodilator FVC in all dosage groups is provided in Tables 8, 9, and FIG. 4. This dexpramipexole improvement in FVC demonstrates an effect on air trapping and mucus plugs, which translates into decreased morbidity and mortality in asthma patients.

TABLE 8
Pre-bronchodilator FVC improvement

| | | | | Difference | |
|---|---|---|---|---|---|
| | | | | versus | |
| | | | Change | Placebo | |
| | | | from | LSM | P-value |
| | | | Baseline | (95% CI) | vs |
| Timepoint | Treatment | n | LSM | [SE] | Placebo |
| Analysis | Placebo | 26 | | | |
| Baseline | | | | | |
| | 75 mg/day | 22 | | | |
| | dexpramipexole | | | | |
| | 150 mg/day | 25 | | | |
| | dexpramipexole | | | | |
| | 300 mg/day | 27 | | | |
| | dexpramipexole | | | | |
| Week 4 | Placebo | 23 | 0.0140 | | |
| | 75 mg/day | 20 | 0.0925 | 0.0785 | 0.4348 |
| | dexpramipexole | | | (−0.121, 0.278) | |
| | | | | [0.100] | |
| | 150 mg/day | 22 | 0.122 | 0.108 | 0.2756 |
| | dexpramipexole | | | (−0.0880, 0.305) | |
| | | | | [0.0987] | |
| | 300 mg/day | 23 | 0.0983 | 0.0844 | 0.3958 |
| | dexpramipexole | | | (−0.112, 0.281) | |
| | | | | [0.0988] | |
| Week 8 | Placebo | 20 | 0.00630 | | |
| | 75 mg/day | 15 | 0.155 | 0.149 | 0.2101 |
| | dexpramipexole | | | (−0.0854, 0.383) | |
| | | | | [0.118] | |
| | 150 mg/day | 21 | 0.152 | 0.146 | 0.1930 |
| | dexpramipexole | | | (−0.0753, 0.368) | |
| | | | | [0.111] | |
| | 300 mg/day | 22 | 0.322 | 0.316 | 0.0056 |
| | dexpramipexole | | | (0.0952, 0.536) | |
| | | | | [0.111] | |
| Week 12 | Placebo | 17 | 0.0410 | | |
| | 75 mg/day | 18 | 0.181 | 0.140 | 0.2629 |
| | dexpramipexole | | | (−0.107, 0.388) | |
| | | | | [0.125] | |
| | 150 mg/day | 21 | 0.0714 | 0.0304 | 0.8027 |
| | dexpramipexole | | | (−0.211, 0.271) | |
| | | | | [0.121] | |
| | 300 mg/day | 24 | 0.257 | 0.216 | 0.0749 |
| | dexpramipexole | | | (−0.0221, 0.453) | |
| | | | | [0.120] | |
| Week 16 or | Placebo | 22 | −0.0326 | | |
| Week 18 | | | | | |
| | 75 mg/day | 17 | 0.297 | 0.329 | 0.0143 |
| | dexpramipexole | | | (0.0674, 0.592) | |
| | | | | [0.132] | |
| | 150 mg/day | 20 | 0.143 | 0.175 | 0.1744 |
| | dexpramipexole | | | (−0.0791, 0.430) | |
| | | | | [0.128] | |
| | 300 mg/day | 25 | 0.267 | 0.299 | 0.0174 |
| | dexpramipexole | | | (0.0539, 0.545) | |
| | | | | [0.124] | |

TABLE 9
Population: Efficacy: Pre-Bronchodilator FVC (L), % change from Baseline

| | | | 75 mg/day | 150 mg/day | 300 mg/day |
|---|---|---|---|---|---|
| | | Placebo | dexpramipexole | dexpramipexole | dexpramipexole |
| | | (N = 27) | (N = 22) | (N = 25) | (N = 28) |
| Timepoint | Statistic | % Change | % Change | % Change | % Change |
| Week 4 | Mean | 1.90 | 2.82 | 3.54 | 1.91 |
| | n | 23 | 20 | 22 | 23 |
| Week 8 | Mean | 2.21 | 7.17 | 5.28 | 11.5 |
| | n | 20 | 15 | 21 | 22 |
| Week 12 | Mean | 2.83 | 7.93 | 3.63 | 8.95 |
| | n | 17 | 18 | 21 | 24 |
| Week 16 or | Mean | 1.55 | 13.4 | 7.33 | 9.11 |
| Week 18 | n | 22 | 17 | 20 | 25 |

Table 10 provides the data for analyzing bronchodilator reversibility, the % improvement in FEV₁ pre/post albuterol bronchodilator (same day). The study entry criteria required ≥12% and ≥200 mL at screening. These values are accepted in the diagnosis of asthma.

At Week 12, the % reversibility is similar between the various study arms and no less in the 3 DEX arms vs. placebo. This indicates that bronchodilator reversibility is preserved with DEX and that the DEX FEV₁ effect is additive to the albuterol bronchodilator effect. Given that albuterol and long acting beta agonist (LABA) drugs all work through the beta-adrenergic receptor, this suggests the value of L ABA therapy with DEX.

TABLE 10
Post-Bronchodilator Percent FEV1 Reversibility (%)
Population: Efficacy
Parameter: Post-Bronchodilator FEV1 Reversibility (mL)

| | | | | | | 75 mg/day | | | 150 mg/day | | | 300 mg/day | |
|---|---|---|---|---|---|---|---|---|---|---|---|---|---|
| | | Placebo | | | | dexpramipexole | | | dexpramipexole | | | dexpramipexole | |
| | | (N = 27) | | | | (N = 22) | | | (N = 25) | | | (N = 28) | |
| | | | | % | | | % | | | % | | | % |
| | Statistic | Observed | Change | Change | Observed | Change | Change | Observed | Change | Change | Observed | Change | Change |
| Analysis | Mean | 388 | | | 343 | | | 356 | | | 427 | | |
| | SE | 58.4 | | | 53.1 | | | 45.1 | | | 52.7 | | |
| | Median | 340 | | | 335 | | | 330 | | | 405 | | |
| | Min | 40.0 | | | −60.0 | | | −70.0 | | | −110 | | |
| | Max | 1570 | | | 1050 | | | 1100 | | | 1080 | | |
| | n | 27 | | | 22 | | | 25 | | | 28 | | |
| Week | Mean | 290 | −102 | 10.8 | 264 | −112 | −58.7 | 279 | −72.3 | −39.8 | 336 | −109 | −19.6 |
| 12 | SE | 48.5 | 70.2 | 27.2 | 49.6 | 69.8 | 39.5 | 34.9 | 62.7 | 33.6 | 52.0 | 78.6 | 21.1 |
| | Median | 290 | −20.0 | −15.4 | 235 | −55.0 | −49.9 | 235 | 0 | −9.68 | 290 | −50.0 | −19.0 |
| | Min | −300 | −1040 | −183 | −50.0 | −790 | −667 | −20.0 | −980 | −700 | −120 | −1020 | −336 |
| | Max | 640 | 230 | 425 | 580 | 400 | 208 | 590 | 490 | 105 | 1140 | 590 | 222 |
| | n | 19 | 19 | 19 | 18 | 18 | 18 | 22 | 22 | 22 | 23 | 23 | 23 |

The study further identified hematologic responders, defined as a subject with a blood eosinophil count of ≤50 cells/μL at week 12. The number of hematologic responders was significantly increased in dexpramipexole treatment groups compared with placebo and was dose-dependent, see Table 10. Further, there was a significant number of subjects with a greater than or equal to 50% decrease in AEC from baseline in dexpramipexole treatment groups compared with placebo, see Table 11.

TABLE 11
Categorical Summary of Blood Absolute Eosinophil Counts During
the Primary Assessment Phase (Population: Efficacy)

| | | | 75 mg/day | | 150 mg/day | | 300 mg/day | |
|---|---|---|---|---|---|---|---|---|
| | | Placebo | dexpramipexole | | dexpramipexole | | dexpramipexole | |
| | | (N = 24) | (N = 22) | | (N = 24) | | (N =2 8) | |
| Timepoint | Category | n (%) | n (%) | | n (%) | | n (%) | |
| Week 12 | AEC ≤50 cells/pL | | 5 | (22.7%) | 9 | (37.5%) | 12 | (42.9%) |
| | [Fisher's exact test | | [0.0172] | | [0.0006] | | [0.0001] | |
| | p-value versus | | | | | | | |
| | placebo | | | | | | | |
| Week 12 | AEC ≥50% reduction | 1 (4.2%) | 11 | (50.07%) | 15 | (62.5%) | 16 | (57.1%) |

| | | | |
|---|---|---|---|
| from Baseline to | [0.00052] | [0.000028] | [0.000058] |
| Week 12 [Fisher's | | | |
| exact test | | | |
| p-value vs | | | |
| placebo] | | | |

n = number of subjects with valid observations, N = number of subjects, Results are presented as n (%) where the percentage is calculated as number of observations in category at timepoint/totalnumber of observations at timepoint *100.

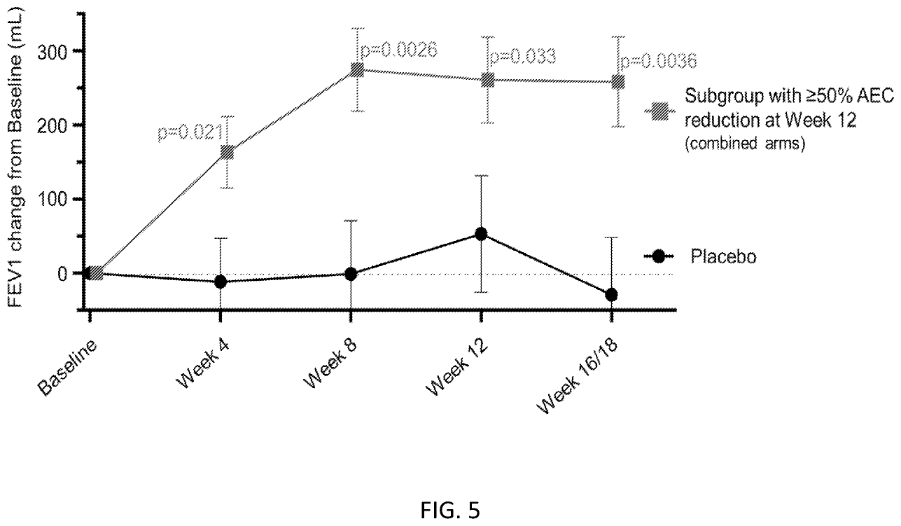

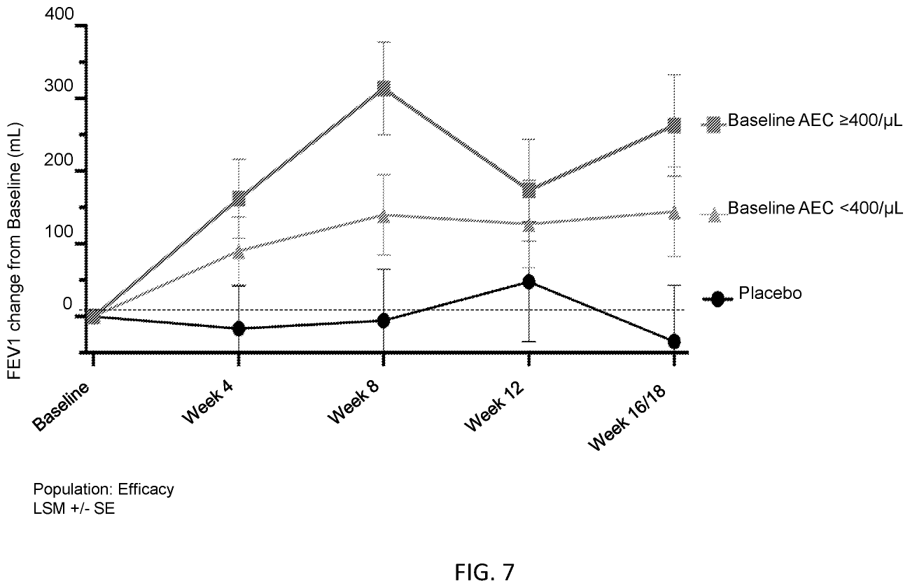

Table 12 and FIG. 5 demonstrates that these hematologic responders showed a significant increase in FEV1 at all post-baseline time points. Improvement in FEV1 was seen in subgroup having <400/μL and even greater FEV1 improvement in the subgroup with baseline AEC ≥400/μL was seen compared placebo shown in FIG. 7.

TABLE 12
Pre-Bronchodilator FEV1 (L) pooled dexpramipexole study arms
(split by ≥ or <50% AEC reduction at Week 12)

| | | n | Baseline | | |
|---|---|---|---|---|---|
| | | | Observed | | |
| Timepoint | Treatment | | Raw mean | | |
| Analysis | Placebo | 26 | 2.05 | | |
| Baseline | | | | | |
| | Pooled dexpramipexole | 42 | 1.97 | | |
| | (≥50% AEC reduction | | | | |
| | at Week 12) | | | | |
| | Pooled dexpramipexole | 33 | 2.14 | | |
| | (<50% AEC reduction | | | | |
| | at Week 12) | | | | |
| | | | | Difference | |
| | | | | versus | |
| | | | Change | Placebo | |
| | | | from | LSM | P-value |
| | | | Baseline | (95% CI) | VS |
| Timepoint | Treatment | n | LSM | [SE] | Placebo |
| Week 4 | Placebo | 23 | −0.0117 | | |
| | Pooled dexpramipexole | 35 | 0.163 | 0.175 | 0.0205 |
| | (≥50% AEC red @ W12) | | | (0.0277, 0.322) | |
| | | | | [0.0741] | |
| | Pooled dexpramipexole | 30 | 0.0742 | 0.0859 | 0.2729 |
| | (<50% AEC red @ W12) | | | (−0.0689, 0.241) | |
| | | | | [0.0778] | |
| Week 8 | Placebo | 20 | −0.00109 | | |
| | Pooled dexpramipexole | 34 | 0.275 | 0.276 | 0.0026 |
| | (≥50% AEC red @ W12) | | | (0.0988, 0.453) | |
| | | | | [0.0890] | |
| | Pooled dexpramipexole | 24 | 0.131 | 0.132 | 0.1743 |
| | (<50% AEC red @ W12) | | | (−0.0593, 0.323) | |
| | | | | [0.0961] | |
| Week 12 | Placebo | 17 | 0.0528 | | |
| | | | Baseline | | |
| | | | Observed | | |
| Timepoint | Treatment | n | Raw mean | | |
| | Pooled dexpramipexole | 36 | 0.261 | 0.208 | 0.0331 |
| | (≥50% AEC red @ W12) | | | (0.0171, 0.399) | |
| | | | | [0.0960] | |
| | Pooled dexpramipexole | 27 | 0.00797 | −0.0448 | 0.6622 |
| | (<50% AEC red @ W12) | | | (−0.248, 0.158) | |
| | | | | [0.102] | |
| Week 16 or | Placebo | 22 | −0.0289 | | |
| Week 18 | | | | | |
| | Pooled dexpramipexole | 36 | 0.258 | 0.287 | 0.0035 |
| | (≥50% AEC red @ W12) | | | (0.0970, 0.476) | |
| | | | | [0.0955] | |
| | Pooled dexpramipexole | 26 | 0.140 | 0.169 | 0.1034 |
| | (<50% AEC red @ W12) | | | (−0.0350, 0.373) | |
| | | | | [0.103] | |

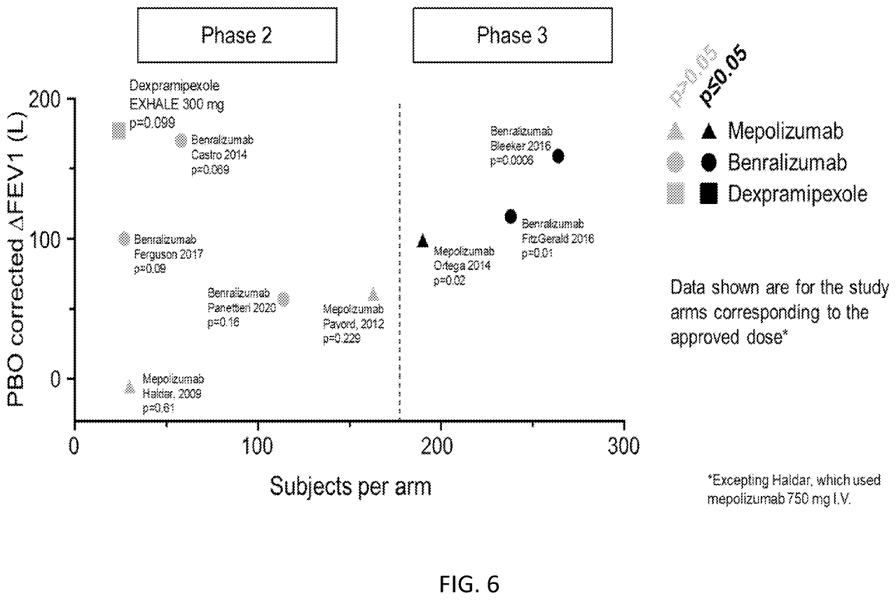

Conclusion: Treatment of moderate to severe asthma with dexpramipexole demonstrated a highly significant, dose-dependent eosinophil lowering response in subjects. This treatment further improved lung function which correlated with the reduction in eosinophils. Surprisingly, approximately 60% of the dexpramipexole treated subjects saw a greater than or equal to 50% reduction in absolute eosinophil count (AEC) and a significant increase in FEV1. Finally, dexpramipexole was well-tolerated across all dose levels. The magnitude and statistical significance of dexpramipexole FEV1 improvement from baseline (shown in Table 5 and FIG. 3) is comparable or greater than that demonstrated in phase 2 clinical trials of benralizumab and mepolizumab. As can be seen in FIG. 6, dexpramipexole's ability to improve FEV1 and effects on lung function are highly competitive with mepolizumab and benralizumab.

As shown in Table 13, administration of dexpramipexole across several dose groups demonstrated improved ACQ-6 scores which indicates both improved treatment of asthma as well as improved asthma control.

TABLE 13
ACQ-6 Numerical Improvement at 12 weeks

| | | Change | Difference | |
|---|---|---|---|---|
| | | from | vs Placebo | P-value |
| | | Baseline | LSM | vs |
| Treatment | n | LSM | (95% Cl) | Placebo |
| Placebo | 25 | −0.391 | | |
| 75 mg/day | 22 | −0.419 | −0.0276 (−0.560, 0.505) | 0.9182 |
| dexpramipexole | | | | |
| 150 mg/day | 24 | −0.437 | −0.0457 (−0.575, 0.484) | 0.8642 |
| dexpramipexole | | | | |
| 300 mg/day | 28 | −0.655 | −0.264 (−0.772, 0.245) | 0.3059 |
| dexpramipexole | | | | |

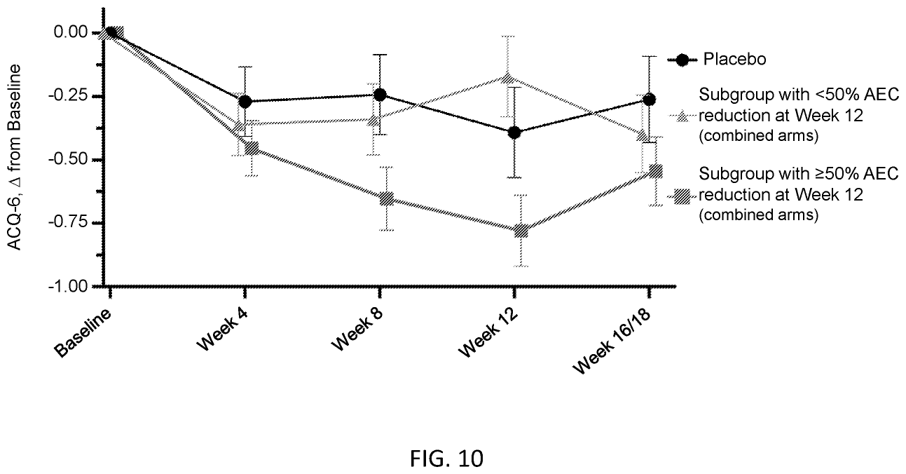

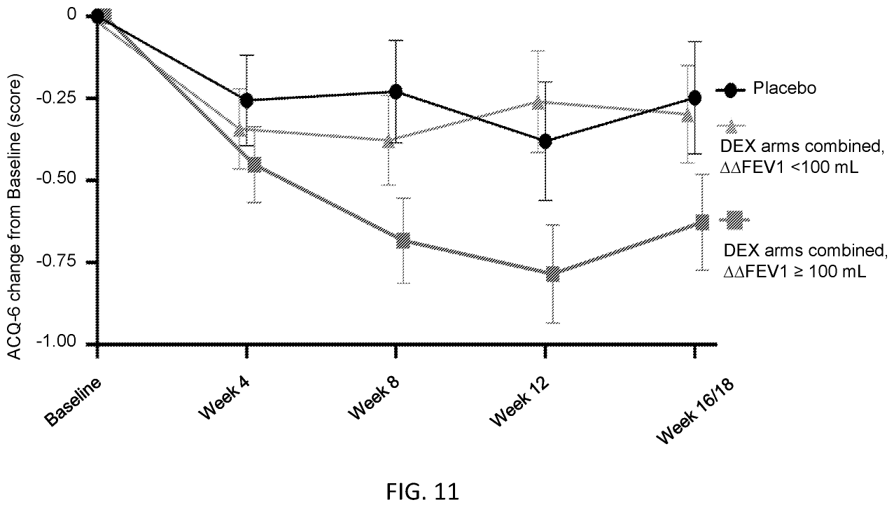

Greater ACQ-6 improvement in subgroup with ≥50% AEC and greater ACQ-6 improvement in subgroup with ΔΔFEV1 ≥100 mL are shown in FIG. 10 and FIG. 11, respectively.

### Example 3: Randomized, Double Blind, Placebo Controlled, Platform Clinical Trial in Patients Stratified into the T2-HIGH Severe Asthma Phenotype (Using Blood Eosinophil Levels

The BEAT-SA T2-HIGH trial (summary set forth in Table 14) will test whether lowering of blood eosinophils in patients receiving dexpramipexole is associated with a reduction in severe exacerbations when compared to matching placebo. 100 participants will be recruited with 50 randomized to receive dexpramipexole and 50 to receive the placebo.

TABLE 14
Summary of Clinical Study Trial

| | |
|---|---|
| Trial Participants | Patients that are both (i) exacerbation prone (≥2 per year; ≥3 days use |
| | of an acute prescription for systemic steroids and/or an acute prescription |
| | for antibiotics due to a worsening of asthma, or admission to hospital/ |
| | A&E) and have (ii) severe asthma according to the ATS/ERS consensus |
| | criterion. Patients eligible for monoclonals are also eligible for BEAT-SA |
| | and can be offered the trial if they wish to participate, following sufficient |
| | explanation that there is a 50% chance of receiving a placebo. Patients that |
| | have failed monoclonal therapy (s) can also be offered the trial after a |
| | period of biologic washout (4 months). |
| Planned Sample Size | T2-HIGH: 100 (50 dexpramipexole, 50 Placebo) |
| Treatment duration | 1 year (follow up) |
| Formulation, | Dexpramipexole 150 mg (or matching placebo) twice daily, orally |
| Dose, Route of | |
| Administration | |
| Primary Objective(s) | (i) Identify patients with severe, exacerbation prone asthma in UK specialist |
| | severe asthma centers. |
| | (ii) Stratify patients using blood eosinophil levels into T2-HIGH phenotype. |
| | (iii) Conduct a 52 week, phase II, randomized double blind |
| | placebo controlled exacerbation trial (RDBPCT) of oral dexpramipexole |
| | (T2-HIGH) or matching placebo. |
| Primary Outcome | Annual rate of severe exacerbations defined as one of more of: |
| Measures | (i) Use of systemic steroid (tablets, suspension or injection) |
| | or an increase in systemic steroids (if stable maintenance) for ≥3 days |
| | (ii) A new prescription of antibiotics for asthma for ≥3 days |
| | (iii) Both (i) and (ii) |
| | (iv) An admission to hospital because of asthma or an emergency department |
| | admission requiring systemic steroids or antibiotics |
| Secondary Outcome | Patients will be assessed for change from baseline at 3, 6, 9 and 12 months for: |
| Measures | Study visits (baseline, 5, 8, 11 and 14) |
| | Juniper six-point Asthma Control Score (ACQ-6) |
| | Juniper Asthma Quality of Life Questionnaire (AQLQ S) |
| | Pre and post bronchodilator FEV1 and FEV1/FVC |
| | Absolute blood eosinophil and neutrophil levels (×10⁹/L) |
| | Nasal eosinophil granule proteins |
| | Fractional exhaled nitric oxide levels (FeNO) |
| | Sino-nasal Outcome Test (SNOT-22) |
| | EuroQOL-5D-5L Questionnaire (Baseline and Final visit) |
| | Work Productivity & Activity Impairment (WPAI) |
| | (Baseline and Final visit) Additional secondary outcomes include: |
| | Time to first severe exacerbation |
| | Annual rate of severe exacerbation events defined by the use of systemic |
| | steroids, antibiotics or both |
| | Adverse events |
| Mechanistic | The mechanistic outcomes for the T2-HIGH trial will be measured at 3 monthly |
| outcomes | intervals (markers of dysbiosis and granulocytic inflammation) and twice |
| | daily (optional use of digital asthma diaries) for CompEX sub-study evaluation. |

Participant Eligibility Criteria

Inclusion Criteria:
Male and female patients aged ≥18 years and <80 years of age.
Capable of giving written informed consent.
Diagnosis of severe asthma as per the ATS/ERS severe asthma guidelines (participants may be included with a lower dose of current ICS than endorsed by the ATS/ERS criteria at the discretion of the investigator if previous high ICS dose had led to side effects).
Stable asthma therapy for at least 1 month before screening.
History of ‘exacerbation prone asthma’ defined as ≥two severe exacerbations within 12 months of MDT initial pre-screening review. Defined as worsening asthma symptoms necessitating one or more of the following:
Use of systemic steroids (tablets, suspension or injection) or an increase in systemic steroids (for those on stable maintenance steroids) for 3 or more days.
A new prescription of antibiotics because of asthma for 3 or more days.
Both (i) and (ii)
An admission to hospital because of asthma, or an emergency department admission requiring systemic steroids (tablets, suspension or injection) or antibiotics.
If any of the above are identified >7 days apart, these would be defined as separate/new exacerbation events.
Willing and able to comply with study protocol requirements.

Exclusion Criteria
Current participation in an investigational drug or device trial at the time of screening.
Patients who are planning to take more than a 21-day consecutive holiday during the trial period.
Have received treatment with biologics such as omalizumab, mepolizumab, reslizumab, benralizumab or dupilumab within four months or five half-lives (whichever is longer) prior to screening.
Recent treatment with bronchial thermoplasty, defined as completion of all thermoplasty treatment sessions within 6 months of screening.
Patients who have been hospitalised or required a new prescription of high-dose (≥10 mg prednisolone/day) oral corticosteroid (OCS) therapy within 4 weeks of the screening visit.
Recent (within 4 weeks of screening) or current lower respiratory tract infection requiring antibiotics (this excludes antibiotics taken for other purposes other than asthma exacerbations).
Acute illness other than asthma which, in the investigator's opinion, may compromise the well-being of the patient or study endpoint assessments at the start of the study.
History of unstable or severe cardiac, hepatic, or renal disease, or other medically significant illness which the investigator believes would be a contraindication to study participation.
Current smoking within the past year or a prior smoking history of ≥15 pack-years (excluding e cigarettes).
Patients with a body mass index (BMI)≤17 or ≥45 kg/m².
History of human immunodeficiency virus (HIV) or hepatitis B or C.
History of malignancy of any organ system (other than localised basal cell carcinoma of the skin), treated or untreated, currently or within the past 5 years, regardless of whether there is evidence of local recurrence or metastases.
History (or suspected history) of alcohol or substance abuse as defined by the Diagnostic and Statistical manual of mental Disorders (5th edition) substance use disorders guidelines within two years of screening.
History of long QT syndrome or whose QTcF interval (Fridericia's) is prolonged >450 msec at screening or baseline.
If female, is pregnant or lactating or intends to become pregnant during the study period where pregnancy is defined as the state of a female after conception and until the termination of gestation, confirmed by a positive hCG laboratory test.
Women of child-bearing potential, defined as all women physiologically capable of becoming pregnant, unless they are using 2 highly effective forms of contraception during dosing of study treatment and for 1 month after treatment.
Males unwilling to use effective methods of contraception during dosing of trial treatment and for at least 3 months after their last dose.
Patients with clinically significant laboratory abnormalities (not associated with the study indication) at screening including (but not limited to):
AST or ALT>2.0×upper limit of normal (ULN) or total bilirubin >1.3×ULN at screening (with the exception of patients with Gilberts syndrome where suitability for inclusion will be left to the discretion of the local investigator).
Estimated Glomerular Filtration Rate (eGFR) by the MDRD equation <55 mL/minute/1.73 m² at screening
Treatment with pramipexole (Mirapex®) within 4 weeks of baseline.
Patients where untreated infection with helminthic parasites is suspected by the clinician.
Concomitant use of drugs known to be associated with significant neutropenia.
Absolute neutrophil count <2.0×10⁹/L at screening, or any documented history of absolute neutrophil count <2.0×10⁹/L on an NHS electronic pathology system available to the local investigator within two years of screening.
Prior history of a neutropenic illness, such as neutropenic sepsis.

Trial Treatment: Participants will be randomized to receive dexpramipexole 150 mg (or matching placebo) be to taken twice daily orally for a maximum of 52 weeks.

Evaluations:

1. Questionnaires

Juniper Asthma Control Six Point Questionnaire (ACQ-6)—Baseline and visits 5, 8, 11 and 14. The ACQ-6 will be used to assess improvements in asthma symptom control (6). The ACQ-6 was originally validated in participants with asthma aged 17 to 90 years (2, 3, and is one of several asthma control measures recommended by GINA Guidelines). The ACQ-6 consists of 6 items: 5 items on symptom assessment, 1 item on rescue bronchodilator use. The ACQ-6 has been fully validated, including a minimal important difference (MID) or smallest change that can be considered clinically important (0.5). The ACQ-6 will be self-administered at the clinic (questions 1-6 only) and it only takes a few minutes to complete. Participants are asked how they have been feeling during the past week and to score each item on a 6-point response scale, where 0 indicates ‘totally controlled’ and 6 indicates ‘severely uncontrolled.’ The total score is calculated as the mean of all questions. The questionnaire should always be completed before any other assessments.

Juniper Asthma Quality of Life Questionnaire (AQLQ S)—Baseline and visits 5, 8, 11 and 14. The disease-specific, standardized version of the asthma quality of life questionnaire (AQLQ) will be used to measure health-related quality of life in trial participants. The measure was originally validated for use in participants with asthma aged 17 to 70 years (7). The AQLQ is a 32-item questionnaire designed to measure functional impairments that are most important to participants with asthma. It consists of 4 domains: symptoms, emotional function, environmental stimuli and activity limitation. Full validation has been demonstrated, including a minimal important difference (MID) or smallest change that can be considered clinically important (0.5). The AQLQ will be self-administered at the clinic and takes about 4 to 5 minutes to complete. Given that the AQLQ will be administered along with the ACQ-6, which has a 1-week recall, participants completing the AQLQ will also be asked to recall their experiences during the past week, and to score each item on a 7-point scale (7=not at all impaired to 1=severely impaired). The AQLQ yields domain-specific scores and a total score, which is the mean response to all 32 questions.

Sino-nasal Outcome Test (SNOT-22)—Baseline and visits 5, 8, 11 and 14. This test highlights the impact of chronic rhinosinusitis on the participant's quality of life through a broad range of health and health-related quality of life issues such as physical problems, functional limitations and emotional consequences (8). There are 22 questions in total and participants are asked to recall and rate their problems over the last 2 weeks. Scoring is separated into six bands: no problem (0), very mild problem (1), mild or slight problem (2), moderate problem (3), severe problem (4) and problem as bad as it can be (5).

EuroQOL-5D-5L Questionnaire—Baseline and visit 14. EuroQOL-5D (EQ-5D-5L) is a standardized assessment of health status to provide a simple, generic measure of health for clinical evaluation (9, 10). It consists of 2 pages covering 5 dimensions (mobility, self-care, usual activities, pain/discomfort and anxiety/depression) and the participant is asked to choose an accompanying statement which best describes how much ease or difficulty they have in each of these dimensions (1 is scored as no problems/pain/anxiety/depression, 5 is scored as unable to/extreme anxiety/depression). The questionnaire also asks the participant to indicate on a scale of 1-100 1=the worst health you can imagine, 100=the best health you can imagine how their health is on that day.

Work Productivity and Activity Impairment Questionnaire (WPAI)—Baseline and visit 14. The WPAI is a 6 item questionnaire used to measure impairment in both paid and unpaid activities. Participants are asked whether they are employed and to recall over the previous 7 days how many hours were missed due to health problems or other reasons, hours actually worked, degree health has affected productivity while working and degree health has affected productivity in regular unpaid activities (0=no effect, 10=severe effect). Results are then converted into percentages.

Lung Function Tests

Fractional Exhaled Nitric Oxide (FeNO)—Baseline and visits 5, 8, 11 and 14. Fractional Exhaled Nitric Oxide (FeNO) is widely accepted as a non-invasive marker for airway inflammation. Fractional exhaled nitric oxide will be measured following the recently published guidelines on standardized techniques including calibration of equipment as appropriate for measuring exhaled Nitric Oxide by ATS and ERS (12). FeNO measurements should be performed PRIOR to spirometry assessments, as spirometric maneuvers have been shown to transiently reduce exhaled NO levels. FeNO measurements have to be performed at the same time of day at the study site for each individual trial participant. Repeated, reproducible exhalations should be performed to obtain one measurements within 10% of each other. Exhaled NO is the mean of these two values. The duration of exhalation must be sufficient (up to 10 seconds) to achieve a stable NO plateau. Allow participants at least 30 seconds of relaxed tidal breathing to rest between repeated exhalations in order not to exhaust the patient. The participant should be seated comfortably with the equipment at the proper height and position. Participants should refrain from eating and drinking at least 2 hours before measurements. Participants should avoid strenuous exercise for 1 hour before measurements. The time of last bronchodilator should be noted, as FeNO levels may vary with the degree of airway obstruction or after bronchodilation. Respiratory tract infections may lead to increased level of exhaled NO in asthma, therefore the infection should be recorded in the participant's medical file (and as an AE in the CRF). Breath-hold results in NO accumulation which causes NO peaks in the exhalations profiles of NO versus time and should therefore be discouraged prior to FeNO.

Spirometry—Baseline and visits 5, 8, 11 and 14. Equipment. Spirometers must meet the specifications and performance criteria recommended in the American Thoracic Society (ATS)/FRS Standardization of Spirometry. Spirometers must have the capacity to print FVC tracings. Spirometry assessments should be performed adequately (in line with the Global Lung Function Initiative [GLI]) and all values should be reported according to the GLI international spirometry reference equation as Z score normalized values. Calibration. The spirometer should be calibrated every morning before any spirometric measurements for the trial are performed. Calibration values will be recorded and stored as source data at the site.

### Example 4: Randomized, Double-Blind, Placebo-Controlled Study of the Effects of Dexpramipexole on Asthma Exacerbations in Severe Eosinophilic Asthma

TABLE 15
Phase 3 Protocol

| Objectives: | Primary Objectives | | |
|---|---|---|---|
| | To evaluate the efficacy of dexpramipexole in reducing asthma exacerbations in subjects with severe eosinophilic asthma | | |
| | Secondary Objectives | | |
| | To evaluate the efficacy of dexpramipexole on pulmonary function and asthma control | | |
| | To evaluate the safety and tolerability of dexpramipexole administered for 52 weeks in subjects with eosinophilic asthma | | |
| | Exploratory Objectives | | |
| | The exploratory objectives of the study include the examination of: | | |
| | The utility of biomarkers to identify dexpramipexole clinical and hematologic responders | | |
| | Evaluate the kinetics of tissue eosinophil lowering | | |
| Endpoints: | Primary Endpoint | | |
| | Severe asthma exacerbations, annualized frequency in the dexpramipexole vs. placebo study arms, as measured from Week 4 | | |
| | to Week 52 | | |
| | A severe asthma exacerbation is defined as worsening of asthma requiring the use of systemic corticosteroids and/or | | |
| | hospitalization and/or Emergency Department (ED) visit. Systemic corticosteroid use is considered evidence of a severe | | |
| | asthma exacerbation for intravenous or oral routes if taken for at least 3 consecutive days; for the IM route, a single dose | | |
| | is sufficient. See Statistics section for justification of the Week 4 to Week 52 analysis window. | | |
| | Secondary Endpoints | | |
| | Pre-bronchodilator FEV1, averaged over Weeks 12, 16, and 21, change from Baseline | | |
| | Severe asthma exacerbations, annualized frequency in the dexpramipexole vs. placebo study arms, as measured from the | | |
| | Baseline visit to Week 52. | | |
| | Pre-bronchodilator FVC, averaged over Weeks 12, 16, and 21, change from Baseline | | |
| | Annualized CompEx event rate, Week 4 to Week 52 | | |
| | Annualized CompEx event rate, Baseline to Week 52 | | |
| | Annualized frequency of exacerbations requiring an ED visit or hospitalization | | |
| | Post-bronchodilator FEV1, averaged over Weeks 12, 16, and 21, change from Baseline | | |
| | Morning peak expiratory flow (PEF), change from Baseline to Week 52 | | |
| | Asthma Control Questionnaire-6 (ACQ), change from Baseline to Week 52 | | |
| | Asthma Quality of Life Questionnaire (AQLQ+12), change from Baseline to Week 52 | | |
| | Incidence and severity of AEs, changes in vital signs, clinical laboratory safety tests, physical examination, body weight, | | |
| | and ECGs | | |
| | Tertiary and Exploratory Endpoints | | |
| | Time to first severe asthma exacerbation | | |
| | Nasal eosinophil peroxidase (EPX) to protein ratio, change from baseline | | |
| | Kinetics of EPX reduction | | |
| | Change from baseline in blood eosinophil count | | |
| | Assessment of screening/baseline biomarkers in peripheral blood to identify dexpramipexole hematological responders | | |
| | The fraction of hematologic responders, defined as subjects with a ≥50% reduction of blood eosinophil count, | | |
| | comparing baseline to the average of all eosinophil counts Week 16 to primary outcome visit | | |
| | In hematologic responders: annualized frequency of severe exacerbations, change from baseline in pre- and post- | | |
| | bronchodilator FEV1, ACQ, and PEF | | |
| | In subjects with screening or baseline blood eosinophil counts ≥0.40 × 10⁹/L | | |
| | (G/L): annualized frequency of severe exacerbations, change from baseline in pre- and post-bronchodilator FEV1, annualized | | |
| | CompEx event rate ACQ, and PEF | | |
| | Whole genome DNA sequencing to discover potential predictive genetic biomarkers to identify hematologic responders | | |
| Study drug: | Oral administration of dexpramipexole study drug tablets BID for 52 weeks at 150 mg/day, 300 mg/day, or placebo | | |
| Number of | 900 subjects (300 per treatment arm). Within each arm subjects will be stratified 2:1, i.e., 200 and 100 subjects, | | |
| subjects: | according to peak blood eosinophil count ≥0.30 × 10⁹/L (G/L) and <0.30 G/L at any time from initial screening | | |
| | through the run-in. | | |
| | | Target number of subjects | |
| | Study arm | High eosinophil stratum | Low eosinophil stratum |
| | Placebo | 200 | 100 |
| | Dexpramipexole 150 mg/day | 200 | 100 |
| | Dexpramipexole 300 mg/day | 200 | 100 |

| | |
|---|---|
| Study | The protocol will enroll symptomatic severe asthmatic subjects 12-74 years as defined by GINA steps 4 and 5. |
| Population: | Subjects must have an FEV1 of <80% o fpredicted at both the Screening and Baseline visits. |
| Rationale | In KNS-760704-AS201, a randomized, double-blind, placebo-controlled dose-ranging trial of dexpramipexole |
| for Study: | in eosinophilic asthma, dexpramipexole demonstrated significant and dose-dependent lowering of blood eosinophil |
| | counts. In the 300 mg/day arm, placebo-corrected FEV1 increased more than 200 mL from baseline, averaged |
| | Week 8 through Week 16/18. Substantial improvements in FEV1 were also seen in the 150 and 75 mg/day arms. |
| | In a post hoc analysis of the mepolizumab Phase 2b and Phase 3 trials, subjects with greater improvements |
| | in FEV1 and FVC had a lower exacerbation rate1. This indicates that the improvement in airflow seen with |
| | dexpramipexole is likely to translate into reduced asthma exacerbations. Annualized exacerbation rate was |
| | chosen at the primary endpoint because exacerbations are an important morbidity of asthma and the most |
| | predictable and responsive clinical endpoint improved by anti-eosinophil therapeutics. |
| Study | This is a randomized, double-blind, placebo-controlled, parallel-group, dose-ranging, multi-center study. |
| Design | Subjects will receive open-label placebo during the Run-in Phase and double-blind study drug during the Primary |
| | Assessment Phase of the study. |
| Duration of | Study Phases |
| Treatment | Run-in Phase: Screening through initiation of Baseline. |
| and Follow- | Subjects will undergo Screening assessments to assess whether they satisfy the eligibility criteria. They will participate |
| up: | in the Run-in Phase (of at least 21 days duration) to confirm a stable asthma medication regimen and to assess |
| | adherence to the dosing schedule, after which eligible subjects will be randomized. |
| | Primary Assessment Phase: Baseline through completion of Week 52. After the collection of all Baseline |
| | assessments, subjects will begin receiving study drug for 52 weeks. The Week 52 visit is the Primary Outcome |
| | Visit for the study. |
| | Eosinophil Recovery Phase: Conclusion of Week 52 assessments through Week 56. |
| | Subjects continuing to the open-label extension study will transition at the Week 52 visit and will not complete |
| | the Week 56 visit. |
| Rationale | The 300 mg/day and 150 mg/day doses were chosen because of the magnitude and statistical significance of the |
| for Dose | blood and tissue eosinophil lowering seen in the KNS-760704-AS201 trial. The large magnitude FEV1 |
| Selection: | improvements noted in the 300 mg/day arm and lower doses, also support this dosing regimen. |
| | Lastly, the excellent safety and tolerability of the above doses in the KNS-760704-AS201 trial and in previous |
| | trials in amyotrophic lateral sclerosis and eosinophil associated diseases further support the use of their use in this trial. |
| Inclusion | To be eligible to participate in this study, candidates must meet the following eligibility criteria at the screening |
| criteria: | visit or at the time point specified in the individual eligibility criterion listed below: |
| | 1. Signed informed consent form |
| | 2. Male or female ≥12 or <75 years of age at randomization Asthma-related criteria |
| | 3. Physician diagnosis of asthma for ≥12 months, based on the Global Initiative for Asthma (GINA) 2020 Guidelines |
| | 4. Asthma requiring daily treatment with, at a minimum, medium dose inhaled corticosteroids in combination |
| | with a long-acting β2 agonist (GINA steps 4-5), for at least 3 months and on a stable dose for at least 1 month |
| | before Screening. Subjects using other controller options without long acting β2 agonist are not eligible for the study. |
| | 5. Bronchodilator reversibility, as evidenced by ≥12% and ≥200 mL improvement in FEV1 15 to 25 minutes |
| | following inhalation of albuterol at Screening. |
| | 6. Pre-bronchodilator FEV1 ≥40% and <80% of predicted at Screening and Baseline. |
| | 7. ACQ-6 ≥ 1.5 at Screening. |
| | 8. Documented history of ≥2 asthma exacerbations requiring treatment with systemic corticosteroids (intramuscular, |
| | intravenous, or oral), in the 12 months prior to screening. |
| | General medical history |
| | 9. Negative pregnancy test at screening |
| | 10. Willing to practice effective contraception during the study and be willing and able to continue contraception |
| | for 1 month (females) or 3 months (males) after the last dose of study treatment. |
| Exclusion | Candidates will be excluded from study entry if any of the following exclusion criteria exist at the screening visit |
| criteria: | or at the time point specified in the individual criterion listed below: |
| | 1. Asthma-related criteria |
| | 1. Hospitalization or emergency room treatment for an acute asthmatic exacerbation within 8 weeks prior to baseline visit |
| | 2. Current diagnosis of allergic bronchopulmonary aspergillosis, eosinophilic granulomatosis with polyangiitis, |
| | eosinophilic gastrointestinal diseases, or hypereosinophilic syndrome, or lung diseases (e.g., chronic obstructive |
| | pulmonary disease, idiopathic pulmonary fibrosis) which may confound interpretation of this trial's findings. |
| | 3. Respiratory infection: Upper or lower respiratory tract, sinus or middle ear infection within the 4 weeks before screening |
| | 2. Prohibited medications |
| | 1. Treatment with a biologic investigational drug in the last 5 months. Treatment with non-biologic investigational |
| | drugs in the previous 30 days or 5-half-lives, whichever is longer. |
| | 2. Treatment with monoclonal antibody therapy, including benralizumab, dupilumab, mepolizumab, reslizumab, omalizumab, |
| | or TNF inhibitors, within 5-half-lives prior to Baseline. |
| | 3. Treatment with pramipexole (Mirapex ®) within 4 weeks of baseline |
| | 4. Treatment with drugs known to prolong QTc interval |
| | 5. Treatment with selected drugs known to have a substantial risk of neutropenia |
| | 3. General medical history |
| | 1. History of malignancy, with the following exceptions. Subjects with basal cell carcinoma, localized squamous cell |
| | carcinoma of the skin, or in-situ carcinoma of the cervix are not excluded, provided that the subject is in remission |
| | and curative therapy was completed ≥12 months prior to Screening. Subjects with other malignancies are not excluded, |
| | provided that the subject is in remission and curative therapy was completed ≥5 years prior to Screening. |
| | 2. History of human immunodeficiency virus (HIV) or chronic infection with hepatitis B or C |
| | 3. History of unstable or severe cardiac, hepatic, or renal disease, or othe rmedically significant illness |
| | 4. Medical or other condition likely to interfere with subject's ability to undergo study procedures, adhere to visit schedule, |
| | or comply with study requirements |
| | 5. Recent history of a parasitic infection or travel to a parasitic endemic area within 6 months prior to screening |
| | 6. Current smoking within the past year or a smoking history of ≥10 pack-years |
| | 7. Known or suspected non-compliance, or alcohol or drug abuse |
| | 8. Unwillingness or inability to follow the procedures outlined in the protocol |
| | 9. Body mass index >40 kg/m² |
| | 4. Clinical safety labs |
| | 1. Absolute neutrophil count <2.00 G/L at screening, or any documented |
| | history of absolute neutrophil count <2.00 G/L |
| | 2. Renal dysfunction, defined as an estimated glomerular filtration rate (eGFR) |
| | <60 mL/min/1.73m² at Screening (using the Chronic Kidney Disease Epidemiology Collaboration [CKD-EPI] formula)) |
| | 5. Cardiac safety |
| | 1. History of long QT syndrome or arrhythmia |
| | 2. ECG showing prolongation of QT/QTc interval >450 ms at screening or pre-dose on day 1. QT/QTc interval calculated |
| | as the mean of triplicate determinations |
| | 3. Clinically important abnormalities in resting ECG that may interfere with the interpretation of QTc interval changes at |
| | screening or pre-dose on Day 1, including any of the following: |
| | a. Heart rate <45 bpm or >100 bpm (average of 3 assessments) |
| | 6. Pregnancy/Lactation |
| | 1. Pregnant women or women breastfeeding |
| Evaluation | Physical examination; vital signs (systolic and diastolic blood pressure, respiratory rate, heart rate, and temperature); |
| of safety: | body weight; 12-lead ECG; clinical laboratory evaluations (hematology, blood chemistry and urinalysis); pregnancy |
| | testing; adverse event monitoring; and concomitant medication monitoringt hroughout the study. |
| | For assessment of potential neutropenia, the neutropenia case report form will be utilized. |
| Evaluation | Rate of asthma exacerbations; Asthma Control Questionnaire (ACQ-6); changes in pulmonary function (FEV1); |
| of efficacy: | Asthma Quality of Life Questionnaire (AQLQ+12); blood eosinophil count. |
| Pharmaco- | Plasma drug concentrations will be obtained from each subject according to the following schedule: |
| kinetic | Baseline visit: prior to dosing with dexpramipexole |
| evaluations: | Week 12 visit: prior to dose |
| | Week 31 visit: prior to dose |

The disclosures of each and every patent, patent application, publication, and accession number cited herein are hereby incorporated herein by reference in their entirety.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

1-72. (canceled)

73. A method of improving the mean forced expiratory volume in 1 second (FEV₁) in a human subject in need thereof comprising orally administering to the subject from about 75 mg to about 300 mg of dexpramipexole or a pharmaceutically acceptable salt thereof.

74. The method of claim 73, wherein FEV₁ is increased by about 5% to about 10% following administration.

75. The method of claim 73, wherein FEV₁ is increased by about 10% to about 20% following administration.

76. The method of claim 73, wherein dexpramipexole or a pharmaceutically acceptable salt thereof is administered once daily.

77. The method of claim 73, wherein dexpramipexole or a pharmaceutically acceptable salt thereof is administered twice per day.

78. The method of claim 73, wherein dexpramipexole or a pharmaceutically acceptable salt thereof is administered at a daily dose of about 150 mg/day.

79. The method of claim 73, wherein dexpramipexole or a pharmaceutically acceptable salt thereof is administered at a daily dose of about 300 mg/day.

80. The method of claim 78, wherein dexpramipexole or a pharmaceutically acceptable salt thereof is administered at a dose of about 75 mg twice per day.

81. The method of claim 79, wherein dexpramipexole or a pharmaceutically acceptable salt thereof is administered at a dose of about 150 mg twice per day.

82. A method of treating human disease with an eosinophilic phenotype in a human subject in need thereof comprising orally administering to the subject from about 75 mg to about 300 mg of dexpramipexole or a pharmaceutically acceptable salt thereof.

83. The method of claim 82, wherein the method results in a reduction of the level of blood absolute eosinophil counts in the subject compared to the level prior to the administration of dexpramipexole to the subject.

84. The method of claim 82, wherein the level of blood absolute eosinophil counts in the subject is reduced by about 90%.

85. The method of claim 82, wherein the level of blood absolute eosinophil counts in the subject is reduced by about 80%.

86. The method of claim 82, wherein the level of blood absolute eosinophil counts in the subject is reduced by about 70%.

87. The method of claim 82, wherein the level of blood absolute eosinophil counts in the subject is reduced by about 60%.

88. The method of claim 82, wherein the level of blood absolute eosinophil counts in the subject is reduced by about 50%.

89. The method of claim 82, wherein dexpramipexole or a pharmaceutically acceptable salt thereof is administered once daily.

90. The method of claim 82, wherein dexpramipexole or a pharmaceutically acceptable salt thereof is administered twice per day.

91. The method of claim 82, wherein dexpramipexole or a pharmaceutically acceptable salt thereof is administered at a daily dose of about 150 mg/day.

92. The method of claim 82, wherein dexpramipexole or a pharmaceutically acceptable salt thereof is administered at a daily dose of about 300 mg/day.

93. The method of claim 91, wherein dexpramipexole or a pharmaceutically acceptable salt thereof is administered at a dose of about 75 mg twice per day.

94. The method of claim 92, wherein dexpramipexole or a pharmaceutically acceptable salt thereof is administered at a dose of about 150 mg twice per day.

95. A method of reducing an exacerbation rate in a human subject exhibiting an eosinophilic phenotype comprising orally administering to the subject from about 75 mg to about 300 mg of dexpramipexole or a pharmaceutically acceptable salt thereof.

96. The method of claim 95, wherein the exacerbation rate is reduced to less than 2 per year.

97. The method of claim 95, wherein dexpramipexole or a pharmaceutically acceptable salt thereof is administered once daily.

98. The method of claim 95, wherein dexpramipexole or a pharmaceutically acceptable salt thereof is administered twice per day.

99. The method of claim 95, wherein dexpramipexole or a pharmaceutically acceptable salt thereof is administered at a daily dose of about 150 mg/day.

100. The method of claim 95, wherein dexpramipexole or a pharmaceutically acceptable salt thereof is administered at a daily dose of about 300 mg/day.

101. The method of claim 99, wherein dexpramipexole or a pharmaceutically acceptable salt thereof is administered at a dose of about 75 mg twice per day.

102. The method of claim 100, wherein dexpramipexole or a pharmaceutically acceptable salt thereof is administered at a dose of about 150 mg twice per day.
